(19) United States
(12) Patent Application Publication
(10) Pub. No.: US 2023/0190941 A1
(43) Pub. Date: Jun. 22, 2023

(54) POLYMERIC LINKERS FOR A GASTRIC RESIDENCE SYSTEM

(71) Applicant: Lyndra Therapeutics, Inc., Watertown, MA (US)

(72) Inventors: Juan Jaramillo MONTEZCO, Watertown, MA (US); Marlene SCHWARZ, Auburndale, MA (US); Rosemary KANASTY, Cambridge, MA (US); David ALTREUTER, Wayland, MA (US); Nicholas DE LA TORRE, Boston, MA (US); Sonia HOLAR, Boston, MA (US); Tyler GRANT, Arlington, MA (US); Craig SIMSES, Quincy, MA (US); Erick PEEKE, Cambridge, MA (US); Erica LAI, Somerville, MA (US)

(21) Appl. No.: 17/774,128

(22) PCT Filed: Nov. 6, 2020
(86) PCT No.: PCT/US2020/059537

§ 371 Date: May 3, 2022

Related U.S. Application Data
(60) Provisional application No. 62/933,226, filed on Nov. 8, 2019.

(51) Int. Cl. A61K 47/59; A61K 47/69; A61K 47/34; A61F 2/04; A61L 31/06; A61P 1/04

(52) U.S. Cl. CPC A61K 47/593; A61F 2/04; A61K 47/34; A61K 47/6937; A61L 31/06; A61P 1/04

(57) ABSTRACT

Gastric residence systems and methods of delivering a drug to an individual using a gastric residence system are described herein. The gastric residence system may include a time-dependent and/or enteric, or dual time-dependent and enteric polymeric linker. In some embodiments, the time-dependent polymeric linker includes PLGA, and optionally PLA or a carrier polymer. The enteric polymeric linker includes an enteric polymeric, and optionally a carrier polymer such as PCL or TPU. The time-dependent polymeric linker may degrade in the stomach of the individual according to a degradation (or flexural modulus loss) profile described herein, and the enteric polymeric linker may degrade in the intestine of the individual another degradation profile described herein (or flexural modulus loss).

### CROSS-REFERENCE TO RELATED APPLICATIONS

This application claims priority benefit of U.S. Provisional Pat. Application No. 62/933,226 filed Nov. 8, 2019. The entire contents of that application are hereby incorporated by reference herein.

### STATEMENT REGARDING FEDERALLY SPONSORED RESEARCH OR DEVELOPMENT

This invention was made with government support under R01 AI131416 awarded by the National Institutes of Health. The government has certain rights in the invention.

### FIELD OF THE INVENTION

The invention relates to gastric residence systems for sustained gastric release of a drug and methods of use thereof.

### BACKGROUND OF THE INVENTION

Gastric residence systems are delivery systems for agents which remain in the stomach for days to weeks, or even over longer periods, during which time drugs or other agents can elute from the systems for absorption in the gastrointestinal tract. Examples of such systems are described in International Patent Application Nos. WO 2015/191920, WO 2015/191925, WO 2017/070612, WO 2017/100367, and WO 2017/205844, each of which is incorporated by reference herein. Over the period of residence, the system releases an agent or agents, such as one or more drugs.

Gastric residence systems are designed to be administered to the stomach of a patient, typically in a capsule which is swallowed or introduced into the stomach by an alternate method of administration (for example, feeding tube or gastric tube). Upon dissolution of the capsule in the stomach, the systems expand or unfold to a size which remains in the stomach and resists passage through the pyloric sphincter over the desired residence period (such as three days, seven days, two weeks, etc.). This requires mechanical stability over the desired residence period. Over the period of residence, the system releases an agent or agents, such as one or more drugs, preferably with minimal burst release, which requires careful selection of the carrier material for the agent in order to provide the desired release profile. While resident in the stomach, the system should not interfere with the normal passage of food or other gastric contents. The system should pass out of the stomach at the end of the desired residence time, and be readily eliminated from the patient. If the system prematurely passes from the stomach into the small intestine, it should not cause intestinal obstruction, and again should be readily eliminated from the patient. These characteristics require careful selection of the materials from which the system is constructed, and the dimensions and arrangement of the system.

The current invention describes advancements in design and manufacture of gastric residence systems, which permit sophisticated tailoring of the materials used in the systems, and the system architecture.

The disclosures of all publications, patents, patent applications and published patent applications referred to herein by an identifying citation are hereby incorporated herein by reference in their entirety.

### SUMMARY OF THE INVENTION

Gastric residence systems comprising a time-dependent polymeric linker and/or an enteric polymeric linker are described herein, along with methods of treating a patient using a gastric residence system. The time-dependent polymeric linker degrades under aqueous conditions, which provides a timer for gastric residence. The enteric polymeric linker quickly degrades in the intestines, and provides a safety mechanism that limits the risk of intestinal blockage.

Described herein is a gastric residence system, comprising one or more first structural members comprising a carrier polymer and an agent, the one or more first structural members attached to a second structural member through a polymeric linker comprising poly(lactic-co-glycolide) (PLGA) and at least one additional linker polymer; wherein the polymeric linker loses 20% or more of its flexural modulus or breaks after incubation in an aqueous solution at pH 1.6 for 30 days at 37° C.; and wherein the gastric residence system is retained in the stomach for a period of at least 24 hours.

In some embodiments of the above gastric residence system, the polymeric linker loses 40% or more of its flexural modulus or breaks after incubation in an aqueous solution at pH 1.6 for 30 days at 37° C. In some embodiments, the polymeric linker loses 60% or more of its flexural modulus or breaks after incubation in an aqueous solution at pH 1.6 for 30 days at 37° C. In some embodiments, the polymeric linker loses 80% or more of its flexural modulus or breaks after incubation in an aqueous solution at pH 1.6 for 30 days at 37° C. In some embodiments, the polymeric linker loses 90% or more of its flexural modulus or breaks after incubation in an aqueous solution at pH 1.6 for 30 days at 37° C. In some embodiments, the polymeric linker loses 40% or more of its flexural modulus or breaks after incubation in an aqueous solution at pH 1.6 for 21 days at 37° C. In some embodiments, the polymeric linker loses 40% or more of its flexural modulus or breaks after incubation in an aqueous solution at pH 1.6 for 21 days at 37° C. In some embodiments, the polymeric linker loses 60% or more of its flexural modulus or breaks after incubation in an aqueous solution at pH 1.6 for 21 days at 37° C. In some embodiments, the polymeric linker loses 80% or more of its flexural modulus or breaks after incubation in an aqueous solution at pH 1.6 for 21 days at 37° C. In some embodiments, the polymeric linker loses 90% or more of its flexural modulus or breaks after incubation in an aqueous solution at pH 1.6 for 21 days at 37° C. In some embodiments, the polymeric linker loses 20% or more of its flexural modulus or breaks after incubation in an aqueous solution at pH 1.6 for 14 days at 37° C. In some embodiments, the polymeric linker loses 40% or more of its flexural modulus or breaks after incubation in an aqueous solution at pH 1.6 for 14 days at 37° C. In some embodiments, the polymeric linker loses 60% or more of its flexural modulus or breaks after incubation in an aqueous solution at pH 1.6 for 14 days at 37° C. In some embodiments, the polymeric linker loses 80% or more of its flexural modulus or breaks after incubation in an aqueous solution at pH 1.6 for 14 days at 37° C. In some embodiments, the polymeric linker loses 90% or more of its flexural modulus or breaks after incubation in an aqueous solution at pH 1.6 for 14 days at 37° C. In some embodiments, the polymeric linker loses 20% or more of its flexural modulus or breaks after incubation in an aqueous solution at pH 1.6 for 7 days at 37° C. In some embodiments, the polymeric linker loses 40% or more of its flexural modulus or breaks after incubation in an aqueous solution at pH 1.6 for 7 days at 37° C. In some embodiments, the polymeric linker loses 60% or more of its flexural modulus or breaks after incubation in an aqueous solution at pH 1.6 for 7 days at 37° C. In some embodiments, the polymeric linker loses 80% or more of its flexural modulus or breaks after incubation in an aqueous solution at pH 1.6 for 7 days at 37° C. In some embodiments, the polymeric linker loses 90% or more of its flexural modulus or breaks after incubation in an aqueous solution at pH 1.6 for 7 days at 37° C. In some embodiments, the polymeric linker loses 20% or more of its flexural modulus or breaks after incubation in an aqueous solution at pH 1.6 for 3 days at 37° C. In some embodiments, the polymeric linker loses 40% or more of its flexural modulus or breaks after incubation in an aqueous solution at pH 1.6 for 3 days at 37° C. In some embodiments, the polymeric linker loses 60% or more of its flexural modulus or breaks after incubation in an aqueous solution at pH 1.6 for 3 days at 37° C. In some embodiments, the polymeric linker loses 80% or more of its flexural modulus or breaks after incubation in an aqueous solution at pH 1.6 for 3 days at 37° C. In some embodiments, the polymeric linker loses 90% or more of its flexural modulus or breaks after incubation in an aqueous solution at pH 1.6 for 3 days at 37° C.

In some embodiments of the gastric residence system, the at least one additional linker polymer comprises polylactic acid (PLA), the carrier polymer, polycaprolactone (PCL), or a thermoplastic polyurethane (TPU). In some embodiments, the carrier polymer comprises PCL and the at least one additional linker polymer comprises PCL. In some embodiments, the carrier polymer comprises TPU and the at least one additional linker polymer comprises a TPU. In some embodiments, the at least one additional linker polymer comprises PLA. In some embodiments, the carrier polymer comprises PCL or TPU.

Also described herein is a gastric residence system, comprising one or more first structural members comprising a carrier polymer and an agent, the one or more first structural members attached to a second structural member through a polymeric linker comprising: (a) poly(lactic-co-glycolide) (PLGA), and (b) polylactic acid (PLA), polycaprolactone (PCL), or a thermoplastic polyurethane (TPU); and wherein the gastric residence system is retained in the stomach for a period of at least 24 hours.

In some embodiments of the gastric residence system, the carrier polymer comprises PCL and the polymeric linker comprises the PLGA and the PCL. In some embodiments, the carrier polymer comprises the TPU and the polymeric linker comprises the PLGA and the TPU. In some embodiments, the polymeric linker comprises the PLGA and the PLA. In some embodiments, the carrier polymer comprises the TPU or the PCL.

In some embodiments of any of the gastric residence systems described above, the PLGA comprises poly(D,L-lactic-co-glycolide) (PDLG). In some embodiments, the PLGA comprises acid-terminated PLGA. In some embodiments, the PLGA comprises ester-terminated PLGA. In some embodiments, the PLGA comprises acid-terminated PLGA and ester-terminated PLGA at a ratio of about 1:9 to about 9:1. In some embodiments, the polymeric linker comprises about 70 wt% or less PLGA. In some embodiments, the polymeric linker comprises between about 30 wt% and about 70 wt% PLGA.

In some embodiments of any of the gastric residence systems described above, the polymeric linker further comprises an enteric polymer. In some embodiments, the enteric polymer comprises hydroxypropyl methylcellulose acetate succinate (HPMCAS). In some embodiments, the polymeric linker further loses 20% or more of its flexural modulus or breaks after incubation in an aqueous solution at pH 6.5 for 3 days at 37° C. In some embodiments, the polymeric linker further loses 40% or more of its flexural modulus or breaks after incubation in an aqueous solution at pH 6.5 for 3 days at 37° C. In some embodiments, the polymeric linker further loses 60% or more of its flexural modulus or breaks after incubation in an aqueous solution at pH 6.5 for 3 days at 37° C. In some embodiments, the polymeric linker further loses 80% or more of its flexural modulus or breaks after incubation in an aqueous solution at pH 6.5 for 3 days at 37° C. In some embodiments, the polymeric linker further loses 20% or more of its flexural modulus or breaks after incubation in an aqueous solution at pH 6.5 for 1 day at 37° C. In some embodiments, the polymeric linker further loses 40% or more of its flexural modulus or breaks after incubation in an aqueous solution at pH 6.5 for 1 day at 37° C. In some embodiments, the polymeric linker further loses 60% or more of its flexural modulus or breaks after incubation in an aqueous solution at pH 6.5 for 1 day at 37° C. In some embodiments, the polymeric linker further loses 80% or more of its flexural modulus or breaks after incubation in an aqueous solution at pH 6.5 for 1 day at 37° C. In some embodiments, the polymeric linker loses its flexural modulus after incubation in an aqueous solution at pH 6.5 for 3 days at 37° C. by more than 10% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 20% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 40% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 60% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

In some embodiments of any of the gastric residence systems described above, the one or more first structural members are attached to the second structural member through the polymeric linker and a second polymeric linker, the second polymeric linker comprising an enteric polymer. In some embodiments, the second polymeric linker further comprises TPU, PCL or PLGA. In some embodiments, the second polymeric linker loses 20% or more of its flexural modulus or breaks after incubation in an aqueous solution at pH 6.5 for 3 days at 37° C.

Further described herein is a gastric residence system, comprising: one or more first structural members comprising a carrier polymer and an agent, the one or more first structural members attached to a second structural member through a polymeric linker comprising: (a) a thermoplastic polyurethane (TPU) or comprising poly(lactic-co-glycolide) (PLGA), and (b) an enteric polymer; wherein the polymeric linker loses 20% or more of its flexural modulus or breaks after incubation in an aqueous solution at pH 6.5 for 3 days at 37° C.; and wherein the gastric residence system is retained in the stomach for a period of at least 24 hours.

In some embodiments, the polymeric linker further loses 40% or more of its flexural modulus or breaks after incubation in an aqueous solution at pH 6.5 for 3 days at 37° C. In some embodiments, the polymeric linker further loses 60% or more of its flexural modulus or breaks after incubation in an aqueous solution at pH 6.5 for 3 days at 37° C. In some embodiments, the polymeric linker further loses 80% or more of its flexural modulus or breaks after incubation in an aqueous solution at pH 6.5 for 3 days at 37° C. In some embodiments, the polymeric linker further loses 20% or more of its flexural modulus or breaks after incubation in an aqueous solution at pH 6.5 for 1 day at 37° C. In some embodiments, the polymeric linker further loses 40% or more of its flexural modulus or breaks after incubation in an aqueous solution at pH 6.5 for 1 day at 37° C. In some embodiments, the polymeric linker further loses 60% or more of its flexural modulus or breaks after incubation in an aqueous solution at pH 6.5 for 1 day at 37° C. In some embodiments, the polymeric linker further loses 80% or more of its flexural modulus or breaks after incubation in an aqueous solution at pH 6.5 for 1 day at 37° C. In some embodiments, the polymeric linker loses its flexural modulus after incubation in an aqueous solution at pH 6.5 for 3 days at 37° C. by more than 10% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 20% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 40% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 60% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

In some embodiments, the carrier polymer comprises TPU and the one or more polymeric linkers comprises TPU. In some embodiments, the polymeric linker further comprises polylactic acid (PLA). In some embodiments, the polymeric linker comprises PLGA. In some embodiments, the PLGA is poly(D,L-lactic-co-glycolide) (PDLG). In some embodiments, the PLGA comprises acid-terminated PLGA. In some embodiments, the PLGA comprises ester-terminated PLGA. In some embodiments, the PLGA comprises acid-terminated PLGA and ester-terminated PLGA at a ratio of about 1:9 to about 9:1. In some embodiments, the polymeric linker comprises about 70 wt% or less PLGA. In some embodiments, the polymeric linker comprises between about 30 wt% and about 70% PLGA.

In some embodiments, the enteric polymer comprises hydroxypropyl methylcellulose acetate succinate (HPMCAS).

In some embodiments, the polymeric linker comprises about 20 wt% to about 80 wt% enteric polymer.

In some embodiments of any of the gastric residence systems described above, the polymeric linker comprises about 0.5 wt% to about 20 wt% plasticizer. In some embodiments, the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

Also described herein is a gastric residence system, comprising: one or more first structural members comprising a carrier polymer and an agent, the one or more first structural members attached to a second structural member through a polymeric linker comprising a linker polymer and about 0.5 wt% to about 20 wt% plasticizer; wherein the gastric residence system is retained in the stomach for a period of at least 24 hours. In some embodiments, the polymeric linker comprises about 0.5% to about 12% plasticizer. In some embodiments, the linker polymer comprises an enteric polymer.

In some embodiments of the gastric residence system described above, the polymeric linker loses 20% or more of their flexural modulus or breaks after incubation in an aqueous solution at pH 6.5 for 3 days at 37° C. In some embodiments, the polymeric linker loses 40% or more of its flexural modulus or breaks after incubation in an aqueous solution at pH 6.5 for 3 days at 37° C. In some embodiments, the polymeric linker loses 60% or more of its flexural modulus or breaks after incubation in an aqueous solution at pH 6.5 for 3 days at 37° C. In some embodiments, the polymeric linker loses 80% or more of its flexural modulus or breaks after incubation in an aqueous solution at pH 6.5 for 3 days at 37° C. In some embodiments, the polymeric linker loses 20% or more of its flexural modulus or breaks after incubation in an aqueous solution at pH 6.5 for 1 day at 37° C. In some embodiments, the polymeric linker loses 40% or more of its flexural modulus or breaks after incubation in an aqueous solution at pH 6.5 for 1 day at 37° C. In some embodiments, the polymeric linker loses 60% or more of its flexural modulus or breaks after incubation in an aqueous solution at pH 6.5 for 1 day at 37° C. In some embodiments, the polymeric linker loses 80% or more of its flexural modulus or breaks after incubation in an aqueous solution at pH 6.5 for 1 day at 37° C. In some embodiments, the polymeric linker loses its flexural modulus after incubation in an aqueous solution at pH 6.5 for 3 days at 37° C. by more than 10% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 20% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 40% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 60% of the loss of its flexural modulus after incubation an aqueous solution at pH 1.6 for 3 days. In some embodiments, the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

In some embodiments of the gastric residence system described above, the enteric polymer comprises hydroxypropyl methylcellulose acetate succinate (HPMCAS).

In some embodiments of the gastric residence system described above, the polymeric linker comprises about 20 wt% to about 80 wt% enteric polymer.

In some embodiments of the gastric residence system described above, the linker polymer comprises the carrier polymer. In some embodiments, the carrier polymer is polycaprolactone (PCL) or a thermoplastic polyurethane (TPU). In some embodiments, the linker polymer comprises polylactic acid (PLA), polycaprolactone (PCL), or a thermoplastic polyurethane (TPU).

In some embodiments of the gastric residence system described above, the linker polymer comprises a time-dependent degradable polymer. In some embodiments, the polymeric linker loses 20% or more of its flexural modulus or breaks after incubation in an aqueous solution at pH 1.6 for 30 days at 37° C. In some embodiments, the polymeric linker loses 40% or more of its flexural modulus or breaks after incubation in an aqueous solution at pH 1.6 for 30 days at 37° C. In some embodiments, the polymeric linker loses 60% or more of its flexural modulus or breaks after incubation in an aqueous solution at pH 1.6 for 30 days at 37° C. In some embodiments, the polymeric linker loses 80% or more of its flexural modulus or breaks after incubation in an aqueous solution at pH 1.6 for 30 days at 37° C. In some embodiments, the polymeric linker loses 90% or more of its flexural modulus or breaks after incubation in an aqueous solution at pH 1.6 for 30 days at 37° C. In some embodiments, the polymeric linker loses 20% or more of its flexural modulus or breaks after incubation in an aqueous solution at pH 1.6 for 21 days at 37° C. In some embodiments, the polymeric linker loses 40% or more of its flexural modulus or breaks after incubation in an aqueous solution at pH 1.6 for 21 days at 37° C. In some embodiments, the polymeric linker loses 60% or more of its flexural modulus or breaks after incubation in an aqueous solution at pH 1.6 for 21 days at 37° C. In some embodiments, the polymeric linker loses 80% or more of its flexural modulus or breaks after incubation in an aqueous solution at pH 1.6 for 21 days at 37° C. In some embodiments, the polymeric linker loses 90% or more of its flexural modulus or breaks after incubation in an aqueous solution at pH 1.6 for 21 days at 37° C. In some embodiments, the polymeric linker loses 20% or more of its flexural modulus or breaks after incubation in an aqueous solution at pH 1.6 for 14 days at 37° C. In some embodiments, the polymeric linker loses 40% or more of its flexural modulus or breaks after incubation in an aqueous solution at pH 1.6 for 14 days at 37° C. In some embodiments, the polymeric linker loses 60% or more of its flexural modulus or breaks after incubation in an aqueous solution at pH 1.6 for 14 days at 37° C. In some embodiments, the polymeric linker loses 80% or more of its flexural modulus or breaks after incubation in an aqueous solution at pH 1.6 for 14 days at 37° C. In some embodiments, the polymeric linker loses 90% or more of its flexural modulus or breaks after incubation in an aqueous solution at pH 1.6 for 14 days at 37° C. In some embodiments, the polymeric linker loses 20% or more of its flexural modulus or breaks after incubation in an aqueous solution at pH 1.6 for 7 days at 37° C. In some embodiments, the polymeric linker loses 40% or more of its flexural modulus or breaks after incubation in an aqueous solution at pH 1.6 for 7 days at 37° C. In some embodiments, the polymeric linker loses 60% or more of its flexural modulus or breaks after incubation in an aqueous solution at pH 1.6 for 7 days at 37° C. In some embodiments, the polymeric linker loses 80% or more of its flexural modulus or breaks after incubation in an aqueous solution at pH 1.6 for 7 days at 37° C. In some embodiments, the polymeric linker loses 90% or more of its flexural modulus or breaks after incubation in an aqueous solution at pH 1.6 for 7 days at 37° C. In some embodiments, the polymeric linker loses 20% or more of its flexural modulus or breaks after incubation in an aqueous solution at pH 1.6 for 3 days at 37° C. In some embodiments, the polymeric linker loses 40% or more of its flexural modulus or breaks after incubation in an aqueous solution at pH 1.6 for 3 days at 37° C. In some embodiments, the polymeric linker loses 60% or more of its flexural modulus or breaks after incubation in an aqueous solution at pH 1.6 for 3 days at 37° C. In some embodiments, the polymeric linker loses 80% or more of its flexural modulus or breaks after incubation in an aqueous solution at pH 1.6 for 3 days at 37° C. In some embodiments, the polymeric linker loses 90% or more of its flexural modulus or breaks after incubation in an aqueous solution at pH 1.6 for 3 days at 37° C.

In some embodiments of the gastric residence system described above, the time-dependent degradable polymer comprises poly(lactic-co-glycolide) (PLGA). In some embodiments, the PLGA comprises poly(D,L-lactic-co-glycolide) (PDLG). In some embodiments, the PLGA comprises acid-terminated PLGA. In some embodiments, the PLGA comprises ester-terminated PLGA. In some embodiments, the PLGA comprises acid-terminated PLGA and ester-terminated PLGA at a ratio of about 1:9 to about 9:1. In some embodiments, the polymeric linker comprises about 70 wt% or less PLGA. In some embodiments, the polymeric linker comprises between about 30% and about 70% PLGA.

In some embodiments of the gastric residnec system describe above, the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

Further described herein is a gastric residence system, comprising: one or more first structural members comprising a carrier polymer and an agent, the one or more first structural members attached to a second structural member through a polymeric linker comprising: (a) a pH-independent degradable polymer, and (b) an enteric polymer; wherein the gastric residence system is retained in the stomach for a period of at least 24 hours.

In some embodiments of the gastric residence system described above, the polymeric linker further comprises the carrier polymer. In some embodiments, the carrier polymer is a TPU or a PCL.

In some embodiments of the gastric residence system described above, the pH-independent degradable polymer comprises PLGA. In some embodiments, the PLGA is poly(D,L-lactic-co-glycolide) (PDLG). In some embodiments, the PLGA comprises acid-terminated PLGA. In some embodiments, the PLGA comprises ester-terminated PLGA. In some embodiments, the PLGA comprises acid-terminated PLGA and ester-terminated PLGA at a ratio of about 1:9 to about 9:1. In some embodiments, the polymeric linker comprises about 70 wt% or less PLGA. In some embodiments, the polymeric linker comprises between about 30 wt% and about 70% PLGA.

In some embodiments of the gastric residence system described above, the enteric polymer comprises hydroxypropyl methylcellulose acetate succinate (HPMCAS).

In some embodiments of the gastric residence system described above, the polymeric linker comprises about 20 wt% to about 80 wt% enteric polymer.

In some embodiments of the gastric residence system described above, the polymeric linker comprises about 0.5 wt% to about 20 wt% plasticizer. In some embodiments, the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

In some embodiments of any of the gastric residence systems described above, materials in the polymeric linker are homogenously blended.

In some embodiments of any of the gastric residence systems described above, the polymeric linker is substantially free of the agent.

In some embodiments of any of the gastric residence systems described above, the polymeric linker further comprises a color-absorbing dye. In some embodiments, the color-absorbing dye comprises iron oxide.

In some embodiments of any of the gastric residence systems described above, the system comprises a plurality of first structural members, wherein each first structural member is attached to the second structural member through a separate polymeric linker; the second structural member is an elastic central member; the gastric residence system is configured to be folded and physically constrained during administration and is configured to assume an open retention shape upon removal of a constraint; and change between the folded shape and the open retention shape is mediated by the elastic central member that undergoes elastic deformation when the residence structure is in the folded shape and recoils when the gastric residence structure assumes the open retention shape. In some embodiments, the gastric residence system is constrained within a capsule configured to degrade with the stomach.

In some embodiments of any of the gastric residence systems described above, the agent is a drug.

In some embodiments of any of the gastric residence systems described above, the second structural member is an elastomer.

In some embodiments of any of the gastric residence systems described above, the second structural member is a central elastomer, and wherein the one or more first structural members are arms that radially project from the central elastomer.

In some embodiments of any of the gastric residence systems described above, the gastric residence system is retained in the stomach for a period of at least 48 hours. In some embodiments, the gastric residence system is retained in the stomach for a period of at least 3 days. In some embodiments, the gastric residence system is retained in the stomach for a period of at least 7 days. In some embodiments, the gastric residence system is retained in the stomach for a period of at least 14 days. In some embodiments, the gastric residence system is retained in the stomach for a period of at least 30 days.

Further described herein is a method of delivering an agent to an individual, comprising deploying the gastric residence system of any of the gastric residence systems described above, within the stomach of the individual. In some embodiments, the individual is a human.

The features of any of the embodiments recited above and herein are combinable with any of the other embodiments recited above and herein where appropriate and practical.

### BRIEF DESCRIPTION OF THE DRAWINGS

FIG. 1A shows an exemplary stellate configuration of a gastric residence system described herein.

FIG. 1B shows another exemplary stellate configuration of a gastric residence system described herein.

FIG. 1C shows an exemplary ring configuration of a gastric residence system described herein.

FIG. 1D shows another exemplary ring configuration of a gastric residence system described herein.

FIG. 2A shows a portion of a gastric residence system that includes an exemplary configuration of a structural member attached to a second structural member through a polymeric linker.

FIG. 2B shows a portion of a gastric residence system that includes another exemplary configuration of a structural member attached to a second structural member through a polymeric linker.

FIG. 2C shows a portion of a gastric residence system that includes another exemplary configuration of a structural member attached to a second structural member through a polymeric linker.

FIG. 2D shows a portion of a gastric residence system that includes an exemplary configuration of a structural member attached to a second structural member through two polymeric linkers.

FIG. 2E shows a portion of a gastric residence system that includes another exemplary configuration of a structural member attached to a second structural member through two polymeric linkers.

FIG. 2F shows a portion of a gastric residence system that includes another exemplary configuration of a structural member attached to a second structural member through two polymeric linkers.

FIG. 2G shows a portion of a gastric residence system that includes another exemplary configuration of a structural member attached to a second structural member through two polymeric linkers.

FIG. 2H shows a portion of a gastric residence system that includes another exemplary configuration of a structural member attached to a second structural member through two polymeric linkers.

FIG. 2I shows a portion of a gastric residence system that includes another exemplary configuration of a structural member attached to a second structural member through two polymeric linkers.

FIG. 2J shows a portion of a gastric residence system that includes another exemplary configuration of a structural member attached to a second structural member through two polymeric linkers.

FIG. 2K shows a portion of a gastric residence system that includes another exemplary configuration of a structural member attached to a second structural member through two polymeric linkers.

FIG. 3 shows an exemplary method of bonding components together to form a gastric residence system.

FIG. 4 shows how the flexural modulus of a material may be tested using the three-point bending test.

FIG. 5 shows the flexural modulus results after incubation various time-dependent polymeric linkers in a fasted state simulated gastric fluid (FaSSGF) at various time points.

FIG. 6 shows the flexural modulus results after incubation various additional time-dependent polymeric linkers in a FaSSGF at various time points.

FIG. 7 shows the flexural modulus results after incubation a time-dependent polymeric linkers containing different amounts of PLGA in a FaSSGF after 3 days and after 18 days.

FIG. 8 shows the flexural modulus results after incubating pH-independent time-dependent polymeric linker in aqueous solutions having different pH values over time.

FIG. 9 compares the flexural modulus of an enteric polymeric linker incubated over time in FaSSGF or a fasted state simulated intestinal fluid (FaSSIF).

FIG. 10 compares the flexural modulus of an enteric polymeric linker containing different amounts of HPMCAS after incubating in FaSSIF over time.

FIG. 11 compares the flexural modulus of an enteric polymeric linker containing different amounts of propylene glycol after incubating in FaSSGF or FaSSIF.

FIG. 12 compares the flexural modulus of a dual time-dependent and enteric polymeric linker containing an enteric polymer (HPMCAS) and a pH-independent degradable polymer (PLGA) after incubation in FaSSGF or FaSSIF over time.

FIG. 13A shows the melt flow index over various materials, including a carrier polymer (base polymer), a time-dependent polymeric linker, and an enteric linker with or within a plasticizer.

FIG. 13B shows the melt flow index of enteric polymeric linker materials with different amounts of plasticizer, as measured at 120° C. and a 2.16 kg load.

FIG. 14A show the change of the tensile strength of a bond after joining an enteric polymeric linker with differing amounts of plasticizer to a time-dependent linker.

FIG. 14B shows the tensile strength of a bond joining an enteric polymeric linker with different amounts of plasticizer to a time-dependent linker. Values indicated by a circle represent materials wherein increased plasticizer replaces both PCL and HPMCAS, and valued indicated by a square represent materials with constant amounts of PCL.

FIG. 15A shows the flexural modulus of various enteric polymeric linker materials after incubating in FaSSGF or FaSSIF.

FIG. 15B shows the flexural modulus of an enteric polymeric linker material containing 60% HPMCAS and 40% TPU after incubating in FaSSGF or FaSSIF as a function of time.

FIG. 16 shows the gastric retention time of a gastric residence system with an enteric polymer mixed with a carrier (i.e., base) polymer at different amounts.

FIG. 17A shows the cyclic incubated nonplanar compressive (CINC) test apparatus holding a stellate gastric residence system.

FIG. 17B illustrates an internal side view of the cyclic incubated nonplanar compressive (CINC) test apparatus, showing the slot into which the stellate arms are placed.

FIG. 18 shows a schematic summarizing the stress relaxation test procedure, indicating the angle that may be measured to track extent of linker deformation. Panel A: before stress relaxation test; Panel B: compression and incubation (for 4 hours); Panel C: measured angle for linker deformation after test.

FIG. 19A and FIG. 19B show stress relaxation “window” test results. For three different linkers described in Table 10, FIG. 19A displays the % difference in arm angle post-window test over time in the stellate arms, while FIG. 19B also includes the % difference in arm angle after recovery. This data demonstrates clear distinctions in stellate and thus linker behavior.

FIG. 20 shows that stellates with a timing linker demonstrate a time-dependent, tunable stress-relaxation behavior. The profile outlined for Timing Linker 1 is associated with a gastric residence of 7.2 ± 3.2 days, and the profile outlined for Timing Linker 2 is associated with a gastric residence of 19.3 ± 3.9 days.

FIG. 21 shows the results of a Stellate Deformation post-Stress Relaxation Test over days in FaSSGF vs. FaSSIF. This data was collected with representative Enteric Linker 1.

FIG. 22A and FIG. 22B illustrate the decay of representative timing and enteric linkers in relevant media. FIG. 22A shows the 3-point flexural modulus of timing linkers 1, 2, and 3 in fasted-state simulated gastric fluid. FIG. 22B shows the 3-point flexural modulus of enteric linkers 1, 2, and 3 in fasted-state simulated gastric fluid or fasted-state simulated intestinal fluid.

### DETAILED DESCRIPTION OF THE INVENTION

Gastric residence systems, methods of making gastric residence systems, and methods of delivering a drug to an individual using a gastric residence system are described herein. The gastric residence system includes one or more drug-containing structural members, which are attached to a second structural member through one or more polymeric linkers. The polymeric linker connecting the drug-containing members to the second member may be an enteric linker (i.e., configured to degrade in an enteric environment), a time-dependent linker (i.e., configured to degrade in the gastric environment over time), or both (i.e., have enteric linker properties and time-dependent linker properties). Some embodiments of the gastric residence system include two polymeric linkers, including an enteric linker and a time-dependent linker, connecting the one or more drug-containing members to the second member. Optionally, a spacer element may separate the first and second polymeric linkers so that they are not in direct contact.

The gastric residence system is deployed in the stomach of an individual, and the drug contained within the drug-containing member diffuses from the gastric residence system into the gastric environment over a gastric residence period, for example over a period of about 24 hours or more. Once the drug has been depleted from the gastric residence system, the gastric residence system must safely exit the gastrointestinal system. The amount of time taken to exit the stomach should be reliable such that the system does not exit the stomach too early (which would allow for insufficient drug delivery during the intended residence period) or too late (which could cause a stomach blockage or over-delivery of the drug). Additionally, the gastric residence system must quickly degrade if it travels to the intestine to avoid an intestinal blockage. Thus, the polymeric linkers of the gastric residence system described herein may be configured to allow for passage from the stomach into the intestines of an individual at the completion of an intended gastric residence time (time-dependent linkers) or allow for the quick degradation and passage of the gastric residence system in the enteric environment (enteric linkers).

Components of the gastric residence system (e.g., drug-containing structural members or other structural members, linkers, etc.) are joined together, for example using a thermal bonding process (e.g., laser beam welding or infrared (IR) welding). This process joins dissimilar materials with different intended functional properties (e.g., drug elution, enteric weakening or breakage, or time-dependent weakening or breakage). However, the materials must be joined with a strong bond that does not unintentionally break during gastric residence. As further discussed herein, the bond strength between a structural element and a polymeric linker may be enhanced by including in the polymeric linker one or more of (1) an amount of plasticizer, (2) a color-absorbing dye, and/or (3) a carrier polymer that is included in the joined structural element.

### Definitions

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

An “agent” is any substance intended for therapeutic, diagnostic, or nutritional use in a patient, individual, or subject. Agents include, but are not limited to, drugs, nutrients, vitamins, and minerals.

“Approximately constant plasma level” refers to a plasma level that remains within a factor of two of the average plasma level (that is, between 50% and 200% of the average plasma level) measured over the period that the gastric residence system is resident in the stomach.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “carrier polymer” is a polymer suitable for blending with an agent, such as a drug, for use in a gastric residence system.

The “diameter” of a particle as used herein refers to the longest dimension of a particle.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “elastic polymer” or “elastomer” is a polymer that is capable of being deformed by an applied force from its original shape for a period of time, and which then substantially returns to its original shape once the applied force is removed.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross-section of the polymeric linker should be positioned vertically, and the flexural modulus should be measured by applying force downward. If the longest line of symmetry for the cross-section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

An “individual,” “patient,” or “subject” refers to a mammal, preferably a human or a domestic animal such as a dog or cat. In a most preferred embodiment, a patient, individual, or subject is a human.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

Two polymers with of the “same polymer type” refers to two polymers made up of the same monomeric unit or monomeric units (in the case of a copolymer) even though the average weight average molecular weight, intrinsic viscosity, monomeric ratios (in the case of a copolymer) or other physical properties may be different.

“Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods disclosed herein can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder.

A “time-dependent” material, such as a time-dependent polymer or a time-dependent linker is a material that degrades over time when the gastric residence system is deployed in the stomach.

A first material described as having an absolute numerical property “within X%” of a corresponding absolute numerical property of second material having a value of “Y” is understood to mean that the absolute numerical property of the first material may fall within the range of (Y - (X% of Y)) to (Y + (X% of Y)). For example, if a material is described has having a flexural modulus after incubation in a first condition within 30% of the flexural modulus of that material after incubation in a second condition, and the material after incubation in the second condition has a flexural modulus of 1000 MPa, the material after incubation in the first condition may have a flexural modulus of 700 MPa to 1300 MPa. A first material described as having an absolute numerical property “more than X%” of a corresponding absolute numerical property of second material having a value of “Y” is understood to mean that the absolute numerical property of the first material is more than (Y + (X% of Y)). For example, if a material is described has having a flexural modulus after incubation in a first condition more than 30% of the flexural modulus of that material after incubation in a second condition, and the material after incubation in the second condition has a flexural modulus of 1000 MPa, the material after incubation in the first condition has a flexural modulus of more than 1300 MPa. Similarly, a first material described as having an absolute numerical property “less than X%” of a corresponding absolute numerical property of second material having a value of “Y” is understood to mean that the absolute numerical property of the first material is more than (Y - (X% of Y)). For example, if a material is described has having a flexural modulus after incubation in a first condition less than 30% of the flexural modulus of that material after incubation in a second condition, and the material after incubation in the second condition has a flexural modulus of 1000 MPa, the material after incubation in the first condition has a flexural modulus of less than 700 MPa.

A first material described as having a relative numerical property “within X%” of a corresponding relative numerical property of a second material having a value of “Y%” is understood to mean that the relative numerical property of the first material may fall within the range of (Y - X)% to (Y + X)%, with the caveat that the lower range may never be lower than 0% and the upper range may never be higher than 100%. For example, if a material is described as losing its flexural modulus after incubation in a first condition within 10% of the loss of the material’s flexural modulus after incubation in a second condition, and the material loses 50% of its flexural modulus after incubation in the second condition, the material after incubation in the first condition may lose between 40% and 60% of its flexural modulus. A first material described as having a relative numerical property “more than X%” of a corresponding relative numerical property of a second material having a value of “Y%” is understood to mean that the relative numerical property of the first material is more than (Y + X)%, with the caveat that the upper relative numerical property may never be higher than 100%. For example, if a material is described as losing its flexural modulus after incubation in a first condition more than 10% of the loss of the material’s flexural modulus after incubation in a second condition, and the material loses 50% of its flexural modulus after incubation in the second condition, the material after incubation in the first condition loses more than 60% of its flexural modulus. Similarly, a first material described as having a relative numerical property “less than X%” of a corresponding relative numerical property of a second material having a value of “Y%” is understood to mean that the relative numerical property of the first material is less than (Y - X)%, with the caveat that the lower relative numerical property may never be lower than 0%. For example, if a material is described as losing its flexural modulus after incubation in a first condition less than 10% of the loss of the material’s flexural modulus after incubation in a second condition, and the material loses 50% of its flexural modulus after incubation in the second condition, the material after incubation in the first condition loses less than 40% of its flexural modulus.

Radial Force Compression Test: A radial force compression test using an iris mechanism may be used to quantify the force required to compress an intact gastric residence system into a configuration small enough to pass through a pylorus. The instrument (i.e., iris tester) used to measure radial force compression is a Blockwise Model TTR2 Tensile Testing Machine with Model RLU124 Twin-Cam™ Radial Compression Station, 60 mm D x 124 mm L. The gastric residence system to be measured should be placed in the iris tester such that the plane of the gastric residence system is parallel to the axis of the iris cylinder. In cases where a stellate-shaped gastric residence system comprising six arms is tested, four arm tips should be placed in contact with the interior wall of the iris tester, where two arms are angled upwards and two arms are angled downwards. Two additional arms should be oriented parallel to the axis of the iris cylinder. As the diameter of the iris mechanism decreases, a radial force is applied to the gastric residence system. A given force measurement is the force required to compress the gastric residence system to the corresponding iris mechanism diameter.

Pullout Force Test: The adhesion strength of a filament for a gastric residence system can be tested using a pullout force test. As described previously, the filament may be attached to a distal end of an arm. In cases where a single filament connects more than two arms, the filament may be connected to the distal end of each arm to prevent translation of the arm along the filament when the gastric residence system is bent by gastric forces. Thus, the pullout force test described herein can quantify the amount of force required to separate the filament from the distal end of an arm. Gastric residence systems having six arms and a filament were prepared and the arms were isolated by cutting the elastomeric core into six parts. The filament was cut between each arm. The tensile force required to pull the filament out of each arm tip was measured using an Instron 3340 Series Universal Testing System by gripping the base of the arm and one end of the filament.

Double Funnel Durability Test: A double funnel test may be used to quantify the durability and/or failure mode of a gastric residence system. The durability of a gastric residence system can help prevent the premature breaking or weakening due to repeated gastric wave/forces (and early passage through the pylorus) of a gastric residence system. To test a gastric residence system using a double funnel test, the system to be tested is gripped at its center (i.e., core) by a ring attached to a linear actuator. The gastric residence system is repeatedly moved upwards and downwards into facing cone-shaped cavities, causing the arms of gastric residence system to bend back and forth with reference to the core. The cone-shaped cavities are facing each other such that the vertex of the cones are opposite each other and the bases of each cone are proximate one another. This upwards and downwards motion is repeated for hundreds of cycles or until the gastric residence system breaks. Different specific failure modes may include a breakage at a connection point (e.g., arm-to-core or first segment-to-second segment) or tearing of the silicone core. The number of cycles to failure and the force required to bend the gastric residence system may be quantified. The test may be performed with the gastric residence system submerged in aqueous media (e.g., simulated gastric fluid) and at body temperature.

Planar Circumferential Bend Durability Test: A planar circumferential test may be used to quantify the durability and/or failure mode of a gastric residence system. In particular, the planar circumferential bend durability test can test a gastric residence system by positioning it onto a puck having four grips each in contact with arms of the gastric residence system. The grips are connected to a rotational actuator that applies force to the arms in a circumferential motion. This motion causes the arms to spread within the plane of the gastric residence system. The motion is repeated for hundreds of cycles or until the gastric residence system breaks. Different specific failure modes may include a breakage at a connection point (e.g., arm-to-core or first segment-to-second segment) or tearing of the silicone core. The number of cycles to failure and the force required to bend the gastric residence system may be quantified. The test may be performed with the gastric residence system submerged in aqueous media (e.g., simulated gastric fluid) and at body temperature.

Melt Flow Index (MFI): The melt flow index (MFI) is a measurement of viscosity at low shear, measured in grams of material that flow through a die in 10 minutes at a set temperature and applied weight. These measurements are performed using a Ray-Ran 6MPCA Advanced Melt Flow System, with a weight of 2.16 kg (but can be with a range of standardized weights) and following Procedure A of ASTM D1238 “Standard Test Method for Melt Flow Rates of Thermoplastics by Extrusion Plastometer.

Tensile Test: An Instron machine having custom-made grips (Figure C) can be used to evaluate the ultimate tensile strength (UTS) of the bond between any combination of stellate components: (1) in a variety of incubation media; (2) at several times of incubation; and (3) at room temperature or body temperature (37-40° C.). A low ultimate tensile strength indicates a potential failure point in the stellate. Using formulation and process optimization, tensile strength can be maximized for ideal stellate performance. For testing stellate arms with a triangular cross-section, custom-made grips can be used having one flat plate and one notched plate. The apex of the triangular arm sits in the notch, in order to distribute the pressure from the plates more evenly across the three lengthwise faces of the triangular arm. Tensile testing was performed using an Instron 3342 Series. A series of hot-melt extruded, thermally bonded equilateral triangular prisms with a 3.33 mm triangular base is gripped using pneumatic actuation. The crosshead moves upward at 5-500 mm/minute depending on the elasticity of the materials tested. The instrument records Force (N) v. Displacement (mm), and the maximum force is divided by the cross-sectional area at the interface to calculate ultimate tensile strength (stress).

Drug Release Rate Test: Release rate of drug is tested in fasted-state simulated gastric fluid (FaSSGF). FaSSGF was prepared as follows, according to the manufacturer’s instructions (biorelevant.com). 975 mL deionized water and 25 mL of 1 N hydrochloric acid were mixed in a 1 L glass media bottle. The pH was adjusted to 1.6 using 1 N HCl or NaOH as needed. 2.0 grams of NaCl was added and mixed in. Just before use, 60 mg of Biorelevant powder was mixed into the solution. The composition of FaSSGF was taurocholate (0.08 mM), phospholipids (0.02 mM), sodium (34 mM), chloride (59 mM). Carrier polymer-agent compositions were formed into drug-loaded polymer arms by blending polymer powder and active pharmaceutical ingredient, and extruding. Arms were coated with release rate-modulating polymer films by dissolving the film polymer in an appropriate solvent, typically ethyl acetate or acetone, and pan-coating or dip-coating the arm in the solution of film polymer. Coated arms are then placed in a vessel containing FaSSGF, incubated at 37° C., and typically sampled at least four times over a seven-day period. Drug content was measured by HPLC.. Samples were stored for no more than 3 days at 4° C. prior to analysis. At each measurement time point, in order to maintain sink conditions, the entire volume of release media was replaced with fresh solution pre-equilibrated to 37° C.

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and WO 2017/205844, each of which are incorporated by reference herein in their entirety.

### Overall Gastric Residence System Configuration

The gastric residence system described herein includes one or more drug-containing structural members that are attached to second structural member through one or more polymeric linkers. The polymeric linkers used to join the drug-containing member to the second structural member may be an enteric polymeric linker, a time-dependent polymeric linker, or a polymeric linker that is both enteric and time-dependent. The gastric residence is configured to remain in the stomach of the individual for a gastric residence period (e.g., at least 24 hours), during which time the time-dependent polymeric linker degrades (i.e., there is a deterioration of the flexural modulus of the time-dependent polymeric linker) or breaks. Weakening or breakage of the time-dependent polymeric linker allows the gastric residence system to pass into the intestine of the individual. Additionally or alternatively, the gastric residence system may include an enteric polymeric linker that degrades (i.e., there is a deterioration of the flexural modulus of the enteric polymeric linker) or breaks in the intestine of the individual after passage from the stomach, either prematurely or after the intended gastric residence period.

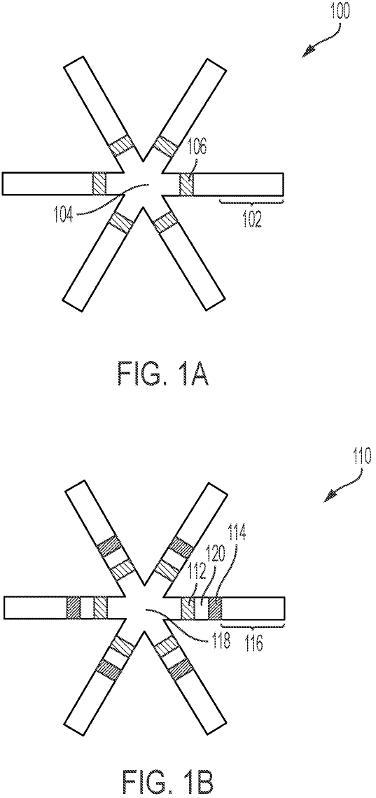

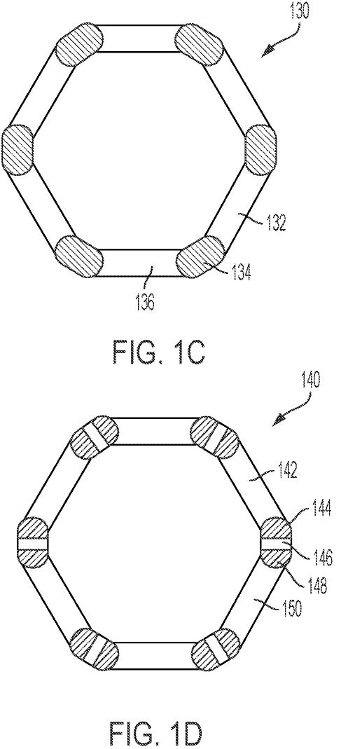

Gastric residence systems can be prepared in different configurations. The “stellate” configuration of a gastric residence system is also known as a “star” (or “asterisk”) configuration. An example of a stellate system 100 is shown schematically in FIG. 1A. Multiple arms (which may also be referred to as “elongate members”) (only one such arms, 102, is labeled for clarity), are affixed to a second structural member, namely a central elastomer 104. The arms 102 are joined to the central elastomer 104 through a polymeric linker 106 (again, only one polymeric linker is labeled for clarity) which serves as a linker region. The polymeric linkers 106 may be enteric linkers or time-dependent linkers, or may have both properties (i.e., are both time-dependent and enteric). This configuration permits the system to be folded or compacted at the central elastomer. When folded, the overall length of the system is reduced by approximately a factor of two, and the system can be conveniently placed in a container such as a capsule or other container suitable for oral administration. When the capsule reaches the stomach, the capsule dissolves, releasing the gastric residence system. The gastric residence system then unfolds into its uncompacted state, which is retained in the stomach for the desired residence period.

FIG. 1B shows another embodiment of a stellate system 110 with two polymeric linkers 112 and 114 joining the arms 116 to the central member 118. The two polymeric linkers may be directly joined together, or may each be directly joined to a coupling member 120 separating the first polymeric linker 112 and the second polymeric linker 114, as shown. A first polymeric linker 112 proximal to the central member 118 may be an enteric linker, and the second polymeric linker 114 distal from the central member 118 may be a time-dependent linker. Alternatively, the first polymeric linker 112 proximal to the central member 118 may be a time-dependent linker, and the second polymeric linker 114 distal from the central member 118 may be an enteric linker. Multiple arms are affixed to and radially project from a central structural member 118.

A stellate system can be described as a gastric residence system for administration to the stomach of a patient, comprising an elastomer component, and a plurality of at least three carrier polymer-agent components (i.e., “arms” or “elongate members”) comprising a carrier polymer and an agent or a salt thereof, attached to the elastomer component, wherein each of the plurality of carrier polymer-agent components is an arm comprising a proximal end and a distal end; wherein the proximal end of each arm is attached to the elastomer component through one or more polymeric linkers and projects radially from the elastomer component, each arm having its distal end not attached to the elastomer component and located at a larger radial distance from the elastomer component than the proximal end. The polymeric linker may be an enteric linker or a time-dependent linker. The arm can be attached to the central elastomer via a one or the polymeric linker or through an additional interfacing polymeric segment. In the stellate configuration, the gastric residence system may have two, three, four, five, six seven, eight, nine, or ten, or more arms. The arms may be equally spaced around the central elastomer; if there are N arms, there will be an angle of about 360/N degrees between neighboring arms.

FIG. 1C shows another possible overall configuration for a gastric residence system 130 in a ring configuration. A first arm 132 is joined to a second elongate segment 136 through a polymeric linkers 134. The second arm may be, for example, an elastomeric member, which allows the ring-shaped system to be configured in a compacted state.

FIG. 1D shows another gastric residence system 140 in a ring configuration. The system 140 includes an arm 142 attached to another arm 150 (and so forth around the ring structure), through a first polymeric linker 144 and a second polymeric linker 148. The first polymeric linker 144 and the second polymeric linker 148 may be directly joined to each other, or may be joined through a coupling member 146. Arm 150 may be the same as arm 142, or may be different. For example, arm 142 may include a carrier polymer and an agent, while arm 150 is an elastomeric member that allows the ring to be configured in a compacted state. In another example, the coupling member 146 may be an elastomeric member, which allows the ring to be configured in a compacted state.

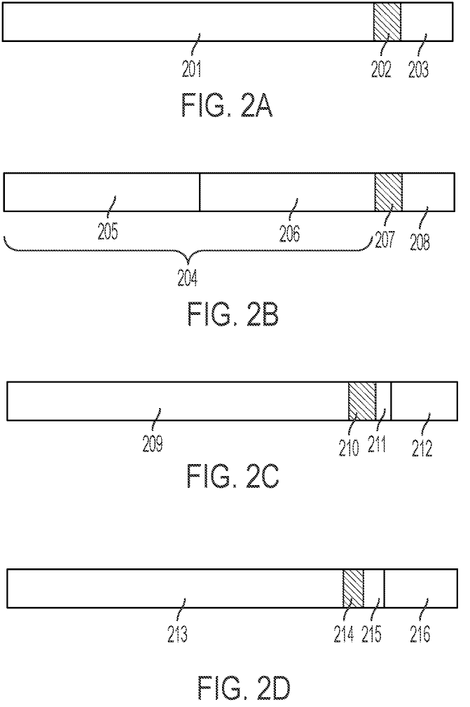

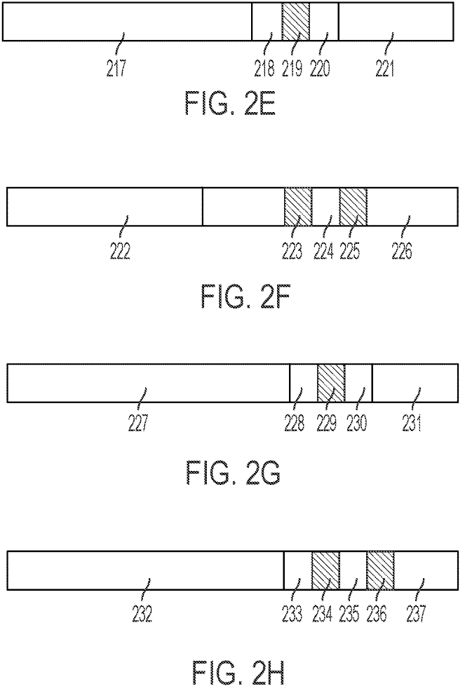

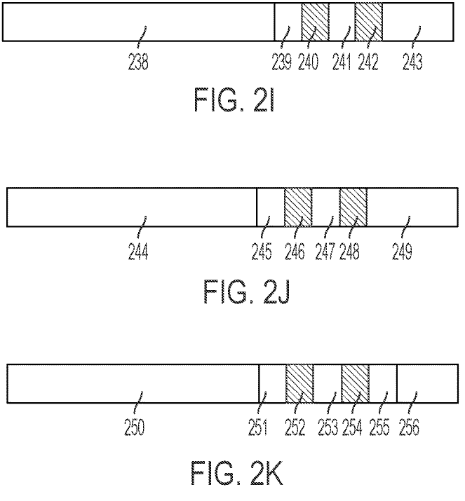

FIGS. 2A-2K illustrate exemplary configurations for attaching a first structural member (such as an arm, which may include an agent and a carrier polymer) to a second structural member (for example, an elastomeric member, such as a central member in a stellate configuration). As further described, the exemplary configurations may include one or two polymeric linkers, and may include zero, one, two, or three coupling members. Further, the arm may include one or more segments, which may include active segments or inactive segments.

FIG. 2A shows a portion of a gastric residence system that includes an arm 201, which is directly attached to polymeric linker 202 (which may be an enteric linker, a time-dependent linker, or a dual time-dependent and enteric linker), which is directly attached to a second structural member 203 (such as a central member or central elastomeric member). The arm 201 can include a carrier polymer and an agent. In some embodiments, the polymeric linker 202 includes the same carrier polymer or same type of carrier polymer as the arm 201.

FIG. 2B shows a portion of a gastric residence system that includes an arm 204, which includes an active segment 205 containing an agent and a carrier polymer and an inactive segment 206 containing the carrier polymer but is substantially free of the agent. The arm 204 is attached to a second structural member 208 (such as a central member, or central elastomeric member) through a polymeric linker 207 (which may be an enteric linker, a time-dependent linker, or a dual time-dependent and enteric linker). The active segment 205 is distal to the polymeric linker 207, and the inactive segment 206 is proximal to and directly attached to the polymeric linker 207, and the polymeric linker 207 is directly attached to the second structural member 208. In some embodiments, the polymeric linker 207 includes the same carrier polymer or same type of carrier polymer as the inactive segment 206.

FIG. 2C shows a portion of a gastric residence system that includes an arm 209, which is directly attached to polymeric linker 210 (which may be an enteric linker, a time-dependent linker, or a dual time-dependent and enteric linker), which is directly attached to a coupling member 211, which is directly attached to second structural member 212 (such as a central member or central elastomeric member). The arm 209 can include a carrier polymer and an agent. In some embodiments, the polymeric linker 210 includes the same carrier polymer or same type of carrier polymer as the arm 209. In some embodiments, the coupling member 211 and the polymeric linker 210 include the same carrier polymer or the same type of carrier polymer as the arm 209.

FIG. 2D shows a portion of a gastric residence system that includes an arm 213, which is directly attached to a first polymeric linker 214 (which may be an enteric linker or a time-dependent linker), which is directly attached to a second polymeric linker 215 (which is an enteric linker if first polymeric linker 214 is a time-dependent linker, or a time-dependent linker if first polymeric linker 214 is an enteric linker), which is directly attached to second structural member 216 (such as a central member or central elastomeric member). The arm 213 can include a carrier polymer and an agent. In some embodiments, the first polymeric linker 214 includes the same carrier polymer or same type of carrier polymer as the arm 213. In some embodiments, the first polymeric linker 214 and the second polymeric linker 215 include the same carrier polymer or the same type of carrier polymer as the arm 213.

FIG. 2E shows a portion of a gastric residence system that includes an arm 217, which is directly attached to a coupling member 218, which is directly attached to a first polymeric linker 219, which is directly attached to a second polymeric linker 220, which his directly attached to a second structural member 221. The arm 217 includes a carrier polymer and an agent. The first polymeric linker 219 may be an enteric linker or a time-dependent linker, and the second polymeric linker 220 may be a time-dependent linker (if the first polymeric linker 219 is an enteric linker) or an enteric linker (if the first polymeric linker 219 is a time-dependent linker). The second structural member 221 may be, for example, a central member (such as a central elastomeric member) of the gastric residence system. The coupling member 218 can include a carrier polymer (which may be the same or same type of carrier polymer in the arm 217), and the first polymeric linker 219 and/or the second polymeric linker 220 may include the same or same type of carrier polymer.

FIG. 2F shows a portion of a gastric residence system that includes an arm 222, which is directly attached to a first polymeric linker 223, which is directly attached to a coupling member 224, which is directly attached to a second polymeric linker 225, which his directly attached to a second structural member 226. The arm 222 includes a carrier polymer and an agent. The first polymeric linker 223 may be an enteric linker or a time-dependent linker, and the second polymeric linker 225 may be a time-dependent linker (if the first polymeric linker 223 is an enteric linker) or an enteric linker (if the first polymeric linker 223 is a time-dependent linker). The second structural member 226 may be, for example, a central member (such as a central elastomeric member) of the gastric residence system. In some embodiments, the first polymeric linker 223 includes the same or same type of carrier polymer present in the arm 222. The coupling member 224 positioned between the first polymeric linker 223 and the second polymeric linker 225 can include a carrier polymer (which may be the same or same type of carrier polymer in the arm 222), and the first polymeric linker 223 and/or the second polymeric linker 225 may include the same or same type of carrier polymer as the coupling member 224.

FIG. 2G shows a portion of a gastric residence system that includes an arm 227, which is directly attached to a first polymeric linker 228, which is directly attached to a second polymeric linker 229, which is directly attached to a coupling member 230, which his directly attached to a second structural member 231. The arm 227 includes a carrier polymer and an agent. The first polymeric linker 228 may be an enteric linker or a time-dependent linker, and the second polymeric linker 229 may be a time-dependent linker (if the first polymeric linker 228 is an enteric linker) or an enteric linker (if the first polymeric linker 228 is a time-dependent linker). The second structural member 231 may be, for example, a central member (such as a central elastomeric member) of the gastric residence system. In some embodiments, the first polymeric linker 228 includes the same or same type of carrier polymer in the arm 227. The coupling member 230 can include a carrier polymer (which may be the same or same type of carrier polymer in the arm 227), and the first polymeric linker 228 and/or the second polymeric linker 229 may include the same or same type of carrier polymer as the coupling member 230.

FIG. 2H shows a portion of a gastric residence system that includes an arm 232, which is directly attached to a first coupling member 233, which is directly attached to a first polymeric linker 234, which is directly attached to a second coupling member 235, which is directly attached to a second polymeric linker 236, which his directly attached to a second structural member 237. The arm 232 includes a carrier polymer and an agent. The first polymeric linker 234 may be an enteric linker or a time-dependent linker, and the second polymeric linker 236 may be a time-dependent linker (if the first polymeric linker 234 is an enteric linker) or an enteric linker (if the first polymeric linker 234 is a time-dependent linker). The second structural member 237 may be, for example, a central member (such as a central elastomeric member) of the gastric residence system. In some embodiments, the first polymeric linker 234 and/or the second polymeric linker 236 includes the same or same type of carrier polymer in the arm 232. In some embodiments, the first coupling member 233 and/or the second coupling member 235 can include a carrier polymer (which may be the same or same type of carrier polymer in the arm 232), and the first polymeric linker 234 and/or the second polymeric linker 236 may include the same or same type of carrier polymer as the first coupling member 233 and/or the second coupling member 235.

FIG. 2I shows a portion of a gastric residence system that includes an arm 238, which is directly attached to a first coupling member 239, which is directly attached to a first polymeric linker 240, which is directly attached to a second polymeric linker 241, which is directly attached to a second coupling member 242, which his directly attached to a second structural member 243. The arm 238 includes a carrier polymer and an agent. The first polymeric linker 240 may be an enteric linker or a time-dependent linker, and the second polymeric linker 241 may be a time-dependent linker (if the first polymeric linker 240 is an enteric linker) or an enteric linker (if the first polymeric linker 240 is a time-dependent linker). The second structural member 243 may be, for example, a central member (such as a central elastomeric member) of the gastric residence system. In some embodiments, the first polymeric linker 240 and/or the second polymeric linker 241 includes the same or same type of carrier polymer in the arm 238. In some embodiments, the first coupling member 239 and/or the second coupling member 242 can include a carrier polymer (which may be the same or same type of carrier polymer in the arm 238), and the first polymeric linker 240 and/or the second polymeric linker 241 may include the same or same type of carrier polymer as the first coupling member 239 and/or the second coupling member 242.

FIG. 2J shows a portion of a gastric residence system that includes an arm 244, which is directly attached to a first polymeric linker 245, which is directly attached to a first coupling member 246, which is directly attached to a second polymeric linker 247, which is directly attached to a second coupling member 248, which his directly attached to a second structural member 249. The arm 244 includes a carrier polymer and an agent. The first polymeric linker 245 may be an enteric linker or a time-dependent linker, and the second polymeric linker 247 may be a time-dependent linker (if the first polymeric linker 245 is an enteric linker) or an enteric linker (if the first polymeric linker 245 is a time-dependent linker). The second structural member 249 may be, for example, a central member (such as a central elastomeric member) of the gastric residence system. In some embodiments, the first polymeric linker 245 and/or the second polymeric linker 247 includes the same or same type of carrier polymer in the arm 244. In some embodiments, the first coupling member 246 and/or the second coupling member 248 can include a carrier polymer (which may be the same or same type of carrier polymer in the arm 244), and the first polymeric linker 245 and/or the second polymeric linker 247 may include the same or same type of carrier polymer as the first coupling member 246 and/or the second coupling member 248.

FIG. 2K shows a portion of a gastric residence system that includes an arm 250, which is directly attached to a first coupling member 251, which is directly attached to a first polymeric linker 252, which is directly attached to a second coupling member 253, which is directly attached to a second polymeric linker 254, which is directly attached to a third coupling member 255, which his directly attached to a second structural member 256. The arm 250 includes a carrier polymer and an agent. The first polymeric linker 252 may be an enteric linker or a time-dependent linker, and the second polymeric linker 254 may be a time-dependent linker (if the first polymeric linker 252 is an enteric linker) or an enteric linker (if the first polymeric linker 252 is a time-dependent linker). The second structural member 256 may be, for example, a central member (such as a central elastomeric member) of the gastric residence system. In some embodiments, the first polymeric linker 252 and/or the second polymeric linker 254 includes the same or same type of carrier polymer in the arm 250. In some embodiments, the first coupling member 251 and/or the second coupling member 253 and/or the third coupling member 255 can include a carrier polymer (which may be the same or same type of carrier polymer in the arm 250), and the first polymeric linker 252 and/or the second polymeric linker 254 may include the same or same type of carrier polymer as the first coupling member 251 and/or the second coupling member 253 and/or the third coupling member 255.

### Polymeric Linkers

The agent-containing structural members are attached to a second structural member (such as a central member, which may be an elastic central member) through one or more linkers. A polymeric linker may directly interface with the agent-containing structural member, or may interface with the agent-containing structural member through a coupling member. Similarly, the polymeric linker may interface directly with the second structural member, or may interface through a coupling member. In an embodiment wherein the agent-containing structural member is connected to the second structural member through two or more polymeric linkers, the polymeric linkers may directly interface with each other, or may interface through a coupling member. One or both of an enteric linker and a time-dependent linkers may be used, or a polymeric linker may function as both an enteric linker and a time-dependent linker.

The polymeric linkers are typically about 100 microns to about 3 millimeter in width, such as about 200 um to about 3000 um, about 300 um to about 3000 um, about 400 um to about 3000 um, about 500 um to about 3000 um, about 600 um to about 3000 um, about 700 um to about 3000 um, about 800 um to about 3000 um, about 900 um to about 3000 um, about 1000 um to about 3000 um, about 1100 um to about 3000 um, about 1200 um to about 3000 um, about 1300 um to about 3000 um, about 1400 um to about 3000 um, about 1500 um to about 3000 um, about 1600 um to about 3000 um, about 1700 um to about 3000 um, about 1800 um to about 3000 um, about 1900 um to about 3000 um, about 2000 um to about 3000 um, about 2100 um to about 3000 um, about 2200 um to about 3000 um, about 2300 um to about 3000 um, about 2400 um to about 3000 um, about 2500 um to about 3000 um, about 2600 um to about 3000 um, about 2700 um to about 3000 um, about 2800 um to about 3000 um, or about 2900 um to about 3000 um; or about 100 um to about 200 um, about 200 um to about 300 um, about 300 um to about 400 um, about 400 um to about 500 um, about 500 um to about 600 um, about 600 um to about 700 um, about 700 um to about 800 um, about 800 um to about 900 um, about 900 um to about 1000 um, about 1000 um to about 1100 um, about 1100 um to about 1200 um, about 1200 um to about 1300 um, about 1300 um to about 1400 um, about 1400 um to about 1500 um, about 1500 um to about 1600 um, about 1600 um to about 1700 um, about 1700 um to about 1800 um, about 1800 um to about 1900 um, about 1900 um to about 2000 um, about 2000 um to about 2100 um, about 2100 um to about 2200 um, about 2200 um to about 2300 um, about 2300 um to about 2400 um, about 2400 um to about 2500 um, about 2500 um to about 2600 um, about 2600 um to about 2700 um, about 2700 um to about 2800 um, about 2800 um to about 2900 um, about 2900 um to about 3000 um. Polymeric linkers can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, about 2000 um, about 2100 um, about 2200 um, about 2300 um, about 2400 um, about 2500 um, about 2600 um, about 2700 um, about 2800 um, about 2900 um, about 3000 um in width, where each value can be plus or minus 50 um (±50 um).

The cross section of the polymeric linker may be round (i.e., circular), elliptical, triangular, square, rectangular, pentagonal, hexagonal, or any other polymeric shape. In some embodiments, the cross-section of the polymeric linker is the same shape as the cross-section of an agent-containing structural member attached to the polymeric linker. In some embodiments, the cross-section of the polymeric linker has a larger area than the cross-section of the agent-containing structural member, a smaller area than the cross-section of the agent-containing structural member, or approximately the same area as the cross-section of the attached agent-containing structural member.

### Time-Dependent Disintegrating Matrices (Time-Dependent Linkers)

A time-dependent linker degrades in a predictable, time-dependent manner under aqueous conditions, such as when the gastric residence system is deployed in the stomach of an individual. The time-dependent polymeric linkers control the residence time of the gastric residence system in the stomach. The time-dependent polymeric linkers are designed to degrade, dissolve, mechanically weaken, or break gradually over time. After the desired residence period, the time-dependent polymeric linker has degraded, dissolved, disassociated, or mechanically weakened, or has broken, to the point where the gastric residence system can pass through the pyloric valve, exiting the gastric environment and entering the small intestine, for eventual elimination from the body.

The time-dependent polymeric linker preferably comprises a pH-independent degradable polymer, which degrades under aqueous conditions in a pH-independent or approximately pH-independent manner. Exemplary pH-independent degradable polymer include PLGA, PLA, PCL, polydioxanone, cellulose, or blends or copolymers thereof. In some embodiments, the pH-independent degradable polymer is PLGA. A pH-independent degradable polymer may be, for example, a polymer that has a flexural modulus after incubation in an aqueous solution, such as fasted state intestinal fluid (FaSSIF), at pH 6.5 for 3 days at 37° C. that is within 30%, within 20%, within 15%, within 10%, or within 5% of the flexural modulus after incubation in an aqueous solution, such as FaSSGF, at pH 1.6 for 3 days at 37° C. In some embodiments, the pH-independent degradable polymer has a flexural modulus after incubation in an aqueous solution, such as fasted state intestinal fluid (FaSSIF), at pH 6.5 for 5 days at 37° C. that is within 30%, within 20%, within 15%, within 10%, or within 5% of the flexural modulus after incubation in an aqueous solution, such as FaSSGF, at pH 1.6 for 5 days at 37° C. In some embodiments, the pH-independent degradable polymer has a flexural modulus after incubation in an aqueous solution, such as fasted state intestinal fluid (FaSSIF), at pH 6.5 for 7 days at 37° C. that is within 30%, within 20%, within 15%, within 10%, or within 5% of the flexural modulus after incubation in an aqueous solution, such as FaSSGF, at pH 1.6 for 7 days at 37° C. In some embodiments, the pH-independent degradable polymer has a flexural modulus after incubation in an aqueous solution, such as fasted state intestinal fluid (FaSSIF), at pH 6.5 for 10 days at 37° C. that is within 30%, within 20%, within 15%, within 10%, or within 5% of the flexural modulus after incubation in an aqueous solution, such as FaSSGF, at pH 1.6 for 10 days at 37° C. In some embodiments, the pH-independent degradable polymer has a flexural modulus after incubation in an aqueous solution, such as fasted state intestinal fluid (FaSSIF), at pH 6.5 for 14 days at 37° C. that is within 30%, within 20%, within 15%, within 10%, or within 5% of the flexural modulus after incubation in an aqueous solution, such as FaSSGF, at pH 1.6 for 14 days at 37° C. In some embodiments, the pH-independent degradable polymer has a flexural modulus after incubation in an aqueous solution, such as fasted state intestinal fluid (FaSSIF), at pH 6.5 for 18 days at 37° C. that is within 30%, within 20%, within 15%, within 10%, or within 5% of the flexural modulus after incubation in an aqueous solution, such as FaSSGF, at pH 1.6 for 18 days at 37° C. In some embodiments, the pH-independent degradable polymer has a flexural modulus after incubation in an aqueous solution, such as fasted state intestinal fluid (FaSSIF), at pH 6.5 for 18 days at 37° C. that is within 30%, within 20%, within 15%, within 10%, or within 5% of the flexural modulus after incubation in an aqueous solution, such as FaSSGF, at pH 1.6 for 21 days at 37° C. In some embodiments, the pH-independent degradable polymer has a flexural modulus after incubation in an aqueous solution, such as fasted state intestinal fluid (FaSSIF), at pH 6.5 for 18 days at 37° C. that is within 30%, within 20%, within 15%, within 10%, or within 5% of the flexural modulus after incubation in an aqueous solution, such as FaSSGF, at pH 1.6 for 28 days at 37° C.

Weakening or degradation of the time-dependent polymeric linker may be measured in references to a loss of the flexural modulus or breakage of the polymeric linker under a given condition (e.g., enteric conditions or gastric conditions). The time-dependent linkers weaken in the gastric environment over a selected gastric residence period, and become sufficiently weak or break such that the gastric residence system can exit the stomach. Stomach conditions may be simulated using an aqueous solution, such as a fasted state simulated gastric fluid (FaSSGF), at a pH of 1.6 and at 37° C. For example, in some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 3 days at 37° C. In some embodiments, the polymeric linker loses a about 60% or more, about 70% or more, about 80% or more, about 90% or more, about 95% or more, or about 99% or more of its flexural modulus or breaks after incubation in an aqueous solution, such as FaSSGF, at pH 1.6 for 5 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 7 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 10 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 14 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 18 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 21 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 24 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 30 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 45 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSGF, at pH 1.6 for 60 days at 37° C.

In certain gastric residence systems, sustained gastric retention is desired, and quick degradation of the time-dependent polymeric linker is less preferred. Accordingly, in some embodiments, the polymeric linker loses about 80% or less, about 70% or less, about 60% or less, about 50% or less, about 40% or less, or about 30% or less of its flexural modulus after incubation in an aqueous solution, such as FaSSGF, at pH 1.6 for 3 days at 37° C. In some embodiments, the polymeric linker loses about 80% or less, about 70% or less, about 60% or less, about 50% or less, about 40% or less, or about 30% or less or less of its flexural modulus after incubation in an aqueous solution, such as FaSSGF, at pH 1.6 for 5 days at 37° C. In some embodiments, the polymeric linker loses about 80% or less, about 70% or less, about 60% or less, about 50% or less, about 40% or less, or about 30% or less of its flexural modulus after incubation in an aqueous solution, such as FaSSGF, at pH 1.6 for 7 days at 37° C. In some embodiments, the polymeric linker loses about 80% or less, about 70% or less, about 60% or less, about 50% or less, about 40% or less, or about 30% or less of its flexural modulus after incubation in an aqueous solution, such as FaSSGF, at pH 1.6 for 10 days at 37° C. In some embodiments, the polymeric linker loses about 80% or less, about 70% or less, about 60% or less, about 50% or less, about 40% or less, or about 30% or less of its flexural modulus after incubation in an aqueous solution, such as FaSSGF, at pH 1.6 for 14 days at 37° C.

The degradation profile of the time-dependent polymeric linker may be configured based on the amount of time-dependent degradable polymer in the time-dependent polymeric linker. For example, a greater amount of poly(lactic-co-glycolide) (PLGA) may result in a greater loss of flexural modulus over an extended gastric residence period, but may retain sufficient structural integrity over a short period of time to retain the gastric residence system in the stomach. By way of example, in some embodiments, the polymeric linker loses about 80% or less, about 70% or less, about 60% or less, about 50% or less, about 40% or less, or about 30% or less of its flexural modulus after incubation in an aqueous solution, such as FaSSGF, at pH 1.6 for 3 days at 37° C., and loses about 60% or more, about 70% or more, about 80% or more, about 90% or more, about 95% or more, or about 99% or more of its flexural modulus or breaks after incubation in an aqueous solution, such as FaSSGF, at pH 1.6 for 7 days at 37° C.

In some embodiments, time-dependent polymeric linkers are pH-independent; that is, the polymeric linker degrades under aqueous conditions in a pH-independent or approximately pH-independent manner. A pH-independent time-dependent polymeric linker may be, for example a time-dependent polymeric linker that has a flexural modulus after incubation in an aqueous solution, such as fasted state intestinal fluid (FaSSIF), at pH 6.5 for 3 days at 37° C. that is within 30%, within 20%, within 15%, within 10%, or within 5% of the flexural modulus after incubation in an aqueous solution, such as FaSSGF, at pH 1.6 for 3 days at 37° C. In some embodiments, the pH-independent time-dependent polymeric linker has a flexural modulus after incubation in an aqueous solution, such as fasted state intestinal fluid (FaSSIF), at pH 6.5 for 5 days at 37° C. that is within 30%, within 20%, within 15%, within 10%, or within 5% of the flexural modulus after incubation in an aqueous solution, such as FaSSGF, at pH 1.6 for 5 days at 37° C. In some embodiments, the pH-independent time-dependent polymeric linker has a flexural modulus after incubation in an aqueous solution, such as fasted state intestinal fluid (FaSSIF), at pH 6.5 for 7 days at 37° C. that is within 30%, within 20%, within 15%, within 10%, or within 5% of the flexural modulus after incubation in an aqueous solution, such as FaSSGF, at pH 1.6 for 7 days at 37° C. In some embodiments, the pH-independent time-dependent polymeric linker has a flexural modulus after incubation in an aqueous solution, such as fasted state intestinal fluid (FaSSIF), at pH 6.5 for 10 days at 37° C. that is within 30%, within 20%, within 15%, within 10%, or within 5% of the flexural modulus after incubation in an aqueous solution, such as FaSSGF, at pH 1.6 for 10 days at 37° C. In some embodiments, the pH-independent time-dependent polymeric linker has a flexural modulus after incubation in an aqueous solution, such as fasted state intestinal fluid (FaSSIF), at pH 6.5 for 14 days at 37° C. that is within 30%, within 20%, within 15%, within 10%, or within 5% of the flexural modulus after incubation in an aqueous solution, such as FaSSGF, at pH 1.6 for 14 days at 37° C. In some embodiments, the pH-independent time-dependent polymeric linker has a flexural modulus after incubation in an aqueous solution, such as fasted state intestinal fluid (FaSSIF), at pH 6.5 for 18 days at 37° C. that is within 30%, within 20%, within 15%, within 10%, or within 5% of the flexural modulus after incubation in an aqueous solution, such as FaSSGF, at pH 1.6 for 18 days at 37° C.

In some embodiments, the time-dependent polymeric linker has an initial flexural modulus of about 100 MPa to about 2500 MPa, such as about 100 MPa to about 2500 MPa, such as about 100 MPa to about 250 MPa, about 250 MPa to about 500 MPa, about 500 mPa to about 750 MPa, about 750 MPa to about 1000 MPa, about 1000 MPa to about 1250 MPa, about 1250 MPa to about 1500 MPa, about 1500 MPa to about 2000 MPa, or about 2000 MPa to about 2500 MPa.

The time-dependent polymeric linker can include poly(lactic-co-glycolide) (PLGA) and at least one additional linker polymer, preferably homogenously mixed together. For example, the PLGA and the additional linker polymer may be homogenously blended together before the mixture is extruded, and the extruded material being cut to a desired size for the polymeric linker. As PLGA is degradable in an aqueous environment, the amount of PLGA in the polymeric linker can affect the time-dependent degradation profile of the polymeric linker, and thus the gastric residence period of the gastric residence system. A higher weight percentage of PLGA in the polymeric linker generally results in faster weakening or degradation of the polymeric linker in an aqueous (e.g., gastric) environment. Similarly, a lower weight percentage of PLGA results in a slower weakening or degradation of the polymeric linker in the aqueous environment. Any amount of PLGA may be used in the polymeric linker, with the amount selected based on the desired degradation profile. For example, in some embodiments, the time-dependent polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) PLGA. In some embodiments, the time-dependent polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) PLGA. In some embodiments, the time-dependent polymeric linker includes about 0.1% to about 10% PLGA, about 10% to about 20% PLGA, about 20% to about 30% PLGA, about 30% to about 40% PLGA, about 40% to about 50% PLGA, about 50% to about 60% PLGA, about 60% to about 70% PLGA, about 70% to about 80% PLGA, about 80% to about 90% PLGA, or about 90% to about 99.9% PLGA. In some embodiments, the time-dependent polymeric linker includes about 30% PLGA or less. In some embodiments, the time-dependent polymer linker includes about 70% PLGA or more. In some embodiments, the time-dependent polymeric linker includes about 30% to about 70% PLGA.

The PLGA in the polymeric linker may include poly(D,L-lactic-co-glycolide) (PDLG), poly(D-lactic-co-glycolide), and/or poly(L-lactic-co-glycolide), although PDLG is preferred. The ratio of lactide monomers to glycolide monomers in the copolymer may range from about 5:95 to about 95:5, such as about 5:95 to about 10:90, about 10:90 to about 20:80, about 20:80 to about 35:65, about 35:65 to about 50:50, about 50:50 to about 65:35, about 65:35 to about 80:20, about 80:20 to about 90:10, or about 90:10 to about 95:5.

The molecular weight of the PLGA also affects the rate of polymer degradation, and thus the rate of loss of the flexural modulus, with higher molecular weight polymers degrading (and thus losing flexural modulus) more slowly. In some embodiments, the mass-weighted molecular weight (M_{w}) of the PLGA is about 5,000 Da to about 250,000 Da, such as about 5,000 Da to about 10,000 Da, about 10,000 to about 20,000 Da, about 20,000 Da to about 30,000 Da, about 30,000 Da to about 50,000 Da, about 50,000 Da to about 100,000 Da, about 100,000 Da to about 150,000 Da, about 150,000 Da to about 200,000 Da, or about 200,000 Da to about 250,000 Da. In some embodiments, the inherent viscosity (as measured in CHCl₃ at 25° C.) of the PLGA is between about 0.1 dl/g to about 1.5 dl/g, such as about 0.1 dl/g to about 0.15 dl/g, about 0.15 dl/g to about 0.25 dl/g, about 0.25 dl/g to about 0.5 dl/g, about 0.5 dl/g to about 0.75 dl/g, about 0.75 dl/g to about 1.0 dl/g, about 1.0 dl/g to about 1.25 dl/g, or about 1.25 dl/g to about 1.5 dl/g.

The amount or ratio of acid-terminated PLGA to ester-terminated PLGA may also affect the degradation or weakening speed of the time-dependent polymeric linker, with a higher proportion of acid-terminated PLGA resulting in a faster degradation or weakening speed compared to a higher proportion of ester-terminated PLGA. In some embodiments, the PLGA comprises, consists essentially of, or consists of acid-terminated PLGA. In some embodiments, the PLGA comprises, consists essentially of, or consists of ester-terminated PLGA. In some embodiments, the PLGA comprises a blend of acid-terminated PLGA and ester-terminated PLGA. For example, in some embodiments, the PLGA comprises a blend of acid-terminated PLGA and ester-terminated PLGA at a ratio of about 1:9 to about 9:1 (such as about 1:9 to about 1:8, about 1:8 to about 1:7, about 1:7 to about 1:6, about 1:6 to about 1:5, about 1:5 to about 1:4, about 1:4 to about 1:3, about 1:3 to about 1:2, about 1:2 to about 1:1, about 1:1 to about 2:1, about 2:1 to about 3:1, about 3:1 to about 4:1, about 4:1 to about 5:1, about 5:1 to about 6:1, about 6:1 to about 7:1, about 7:1 to about 8:1, about 8:1 to about 9:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, or about 1:9. In some embodiments, the PLGA comprises a blend of acid-terminated PLGA and ester-terminated PLGA at a ratio of about 1:1.

In some embodiments, the PLGA of the time-dependent polymeric linker comprises acid-terminated poly(D,L-lactic-co-glycolide) with a ratio of lactide monomers to glycolide monomers of about 50:50 and an inherent viscosity between 0.16 dl/g and 0.24 dl/g (such as the PLGA sold under the tradename Purasorb® PDLG 5002A or Purasorb® PDLG 5002A Y, each available from Corbion). In some embodiments, the PLGA of the time-dependent polymeric linker comprises poly(D,L-lactic-co-glycolide) with a ratio of lactide monomers to glycolide monomers of about 50:50 and an inherent viscosity between 0.16 dl/g and 0.24 dl/g (such as the PLGA sold under the tradename Purasorb® PDLG 5002, available from Corbion). In some embodiments, the PLGA of the time-dependent polymeric linker comprises poly(D,L-lactic-co-glycolide) with a ratio of lactide monomers to glycolide monomers of about 50:50 (such as the PLGA sold under the tradename Purasorb® PDLG 5004, available from Corbion). In some embodiments, the PLGA of the time-dependent polymeric linker comprises an acid-terminated poly(D,L-lactic-co-glycolide) with a ratio of lactide monomers to glycolide monomers of about 50:50 (such as the PLGA sold under the tradename Purasorb® PDLG 5004A, available from Corbion). In some embodiments, the PLGA of the time-dependent polymeric linker comprises poly(D,L-lactic-co-glycolide) with a ratio of lactide monomers to glycolide monomers of about 50:50 and an inherent viscosity between 0.8 dl/g and 1.2 dl/g (such as the PLGA sold under the tradename Purasorb® PDLG 5010, available from Corbion). In some embodiments, the PLGA of the time-dependent polymeric linker comprises poly(D,L-lactic-co-glycolide) with a ratio of lactide monomers to glycolide monomers of about 55:45 and an inherent viscosity between 0.4 dl/g and 0.6 dl/g (such as the PLGA sold under the tradename Purasorb® PDLG 5505G, available from Corbion). In some embodiments, the PLGA of the time-dependent polymeric linker comprises acid-terminated poly(D,L-lactic-co-glycolide) with a ratio of lactide monomers to glycolide monomers of about 75:25 and an inherent viscosity between 0.16 dl/g and 0.24 dl/g (such as the PLGA sold under the tradename Purasorb® PDLG 7502A, available from Corbion). In some embodiments, the PLGA of the time-dependent polymeric linker comprises poly(D,L-lactic-co-glycolide) with a ratio of lactide monomers to glycolide monomers of about 75:25 and an inherent viscosity between 0.16 dl/g and 0.24 dl/g (such as the PLGA sold under the tradename Purasorb® PDLG 7502, available from Corbion). In some embodiments, the PLGA of the time-dependent polymeric linker comprises a poly(D,L-lactic-co-glycolide) with a ratio of lactide monomers to glycolide monomers of about 75:25 and an inherent viscosity between 0.65 dl/g and 0.95 dl/g (such as the PLGA sold under the tradename Purasorb® PDLG 7507 Y, available from Corbion). In some embodiments, the PLGA of the time-dependent polymeric linker comprises a poly(D,L-lactic-co-glycolide) with a ratio of lactide monomers to glycolide monomers of about 75:25 and an inherent viscosity between 0.56 dl/g and 0.84 dl/g (such as the PLGA sold under the tradename Purasorb® PDLG 7507, available from Corbion). In some embodiments, the PLGA of the time-dependent polymeric linker comprises a poly(D,L-lactic-co-glycolide) with a ratio of lactide monomers to glycolide monomers of about 75:25 and an inherent viscosity between 0.85 dl/g and 1.05 dl/g (such as the PLGA sold under the tradename Purasorb® PDLG 7510, available from Corbion). In some embodiments, the PLGA of the time-dependent polymeric linker comprises a poly(D,L-lactic-co-glycolide) with a ratio of lactide monomers to glycolide monomers of about 65:35 and an inherent viscosity between 0.32 dl/g and 0.44 dl/g (such as the PLGA sold under the tradename Resomer® RG 653 H, available from Evonik). In some embodiments, the PLGA of the time-dependent polymeric linker comprises a mixture of two or more of the above PDLG polymers. By way of example, in some embodiments, the PLGA of the time-dependent polymeric linker comprises a mixture of (a) poly(D,L-lactic-co-glycolide) with a ratio of lactide monomers to glycolide monomers of about 50:50 (such as the PLGA sold under the tradename Purasorb® PDLG 5004, available from Corbion), and (b) acid-terminated poly(D,L-lactic-co-glycolide) with a ratio of lactide monomers to glycolide monomers of about 50:50 (such as the PLGA sold under the tradename Purasorb® PDLG 5004A, available from Corbion).

The one or more additional linker polymers included in the polymer linker is preferably homogenously mixed with the PLGA. In some embodiments, the one or more additional linker polymers are miscible with the PLGA. The one or more additional linker polymers may be a non-degradable polymer (that is, not degradable or in the gastric or enteric environment, or an aqueous solution of pH 1.6 (representing the gastric environment) or pH 6.5 (representing the enteric environment), and is optionally present in the time-dependent polymeric linker is an amount such that the time-dependent polymeric linker does not break during the gastric residence period.

Bonding of the polymeric linker to a directly adjacent member may be improved if at least one polymer is common to both the adjacent member and the time-dependent polymeric linker. That is, one of the one or more additional linker polymers in the time-dependent linker may be the same (or the same polymer type) as at least one polymer in a directly adjacent component (or, optionally, both directly adjacent components) of the gastric residence system. For example, if the time-dependent polymeric linker is bonded directly to a structural member comprising a carrier polymer, in some embodiments the one or more additional linker polymers also includes the carrier polymer (in addition to the PLGA in the time-dependent polymeric linker) at the same or different concentration. Exemplary carrier polymers include, but are not limited to, polylactic acid (PLA), polycaprolactone (PCL), and a thermoplastic polyurethane (TPU), among others described herein.

In some embodiments, the one or more additional linker polymers is PLA, for example a PLA as described herein in reference to carrier polymers. In some embodiments, the time-dependent polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) PLA. In some embodiments, the time-dependent polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) PLA. In some embodiments, the time-dependent polymeric linker includes about 0.1% to about 10% PLA, about 10% to about 20% PLA, about 20% to about 30% PLA, about 30% to about 40% PLA about 40% to about 50% PLA, about 50% to about 60% PLA, about 60% to about 70% PLA, about 70% to about 80% PLA, about 80% to about 90% PLA, or about 90% to about 99.9% PLA. In some embodiments, the time-dependent polymeric linker includes about 30% PLA or less. In some embodiments, the time-dependent polymer linker includes about 70% PLA or more. In some embodiments, the time-dependent polymeric liker includes about 30% to about 70% PLA. The PLGA may be further included with the PLA, and can make up to the balance of the time-dependent polymeric linker, although additional agents (such as a plasticizer, a coloring agent, or other agent may be further included).

In some embodiments, the time-dependent polymeric linker includes 10 to 90 %, 20 to 80 %, 30 to 70 %, 40 to 60 %, 45 to 55 %, 48 to 52 %, or 50 % (by weight) PLA. In some embodiments, the time-dependent polymeric linker includes 10 to 50 %, 20 to 40 %, 25 to 35 %, 28 to 32 %, or 30 % (by weight) PLA. In some embodiments, the time-dependent polymeric linker includes 10 to 40 %, 15 to 35 %, 20 to 28 %, 22 to 26 %, or 24 % (by weight) PLA.

In some embodiments, the one or more additional linker polymers comprises a PCL. The time-dependent polymeric linker may be directly joined or bonded to another member of the gastric residence system (such as the structural member comprising the drug and the carrier polymer, a coupling member, the enteric polymeric linker, or a central structural member), which may also include a PCL, which may be the same PCL in the time-dependent polymeric linker or a different PCL as the one in the polymeric linker, and which may be at the same concentration or a different concentration. A different PCL in the time-dependent polymeric linker and the other member directly joined or bonded to the time-dependent linker may differ, for example, in the weight-average molecular weight of the PCL, the inherent viscosity of the PCL, or the proportions of PCL (for example, when a blend of two or more PCL polymers are used).

In some embodiments, the time-dependent polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) PCL. In some embodiments, the time-dependent polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) PCL. In some embodiments, the time-dependent polymeric linker includes about 0.1% to about 10% PCL, about 10% to about 20% PCL, about 20% to about 30% PCL, about 30% to about 40% PCL about 40% to about 50% PCL, about 50% to about 60% PCL, about 60% to about 70% PCL, about 70% to about 80% PCL, about 80% to about 90% PCL, or about 90% to about 99.9% PCL. In some embodiments, the time-dependent polymeric linker includes about 30% PLA or less. In some embodiments, the time-dependent polymer linker includes about 70% PLA or more. In some embodiments, the time-dependent polymeric liker includes about 30% to about 70% PCL. The PLGA may be further included with the PCL, and can make up to the balance of the time-dependent polymeric linker, although additional agents (such as a plasticizer, a coloring agent, or other agent may be further included).

In some embodiments, the one or more additional linker polymers comprises a TPU. The time-dependent polymeric linker may be directly joined or bonded to another member of the gastric residence system (such as the structural member comprising the drug and the carrier polymer, a coupling member, the enteric polymeric linker, or a central structural member), which may also include a TPU, which may be the same TPU in the time-dependent polymeric linker or a different TPU as the one in the polymeric linker, and which may be at the same concentration or a different concentration. A different TPU in the time-dependent polymeric linker and the other member directly joined or bonded to the time-dependent linker may differ, for example, in the weight-average molecular weight of the TPU, the inherent viscosity of the TPU, or the proportions of TPU (for example, when a blend of two or more TPU polymers are used). Suitable commercially-available TPU polymers may include Pathway™ TPU polymers (The Lubrizol Corporation), Tecoflex™ (The Lubrizol Corporation), Tecophilic™ (The Lubrizol Corporation), Carbothane™ (The Lubrizol Corporation), Texin® (Covestro), and NEUSoft™ (PolyOne).

In some embodiments, the time-dependent polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) TPU. In some embodiments, the time-dependent polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) TPU. In some embodiments, the time-dependent polymeric linker includes about 0.1% to about 10% TPU, about 10% to about 20% TPU, about 20% to about 30% TPU, about 30% to about 40% TPU about 40% to about 50% TPU, about 50% to about 60% TPU, about 60% to about 70% TPU, about 70% to about 80% TPU, about 80% to about 90% TPU, or about 90% to about 99.9% TPU. In some embodiments, the time-dependent polymeric linker includes about 30% TPU or less. In some embodiments, the time-dependent polymer linker includes about 70% TPU or more. In some embodiments, the time-dependent polymeric liker includes about 30% to about 70% TPU. In some embodiments, the time-dependent polymeric liker includes about 30% to about 70% PLA. The PLGA may be further included with the TPU, and can make up to the balance of the time-dependent polymeric linker, although additional agents (such as a plasticizer, a color-absorbing dye, or other agent may be further included).

The time-dependent polymeric linker may further include one or more plasticizers, which can aid in cutting an extruded polymeric linker material to a desired size and aid in bonding or attaching the time-dependent polymeric linker to other components of the gastric residence system. Exemplary plasticizers include, but are not limited to, propylene glycol, polyethylene glycol (PEG), triethyl butyl citrate (TBC), dibutyl sebacate (DBS), triacetin, triethyl citrate (TEC), a poloxamer (e.g., Poloxamer 407, or “P407”), or D-α-tocopheryl polyethylene glycol succinate. The term “polyethylene glycol” is used interchangeably herein with the terms “polyethylene oxide” and “PEO.” In some embodiments, the molecular weight of the polyethylene glycol is about 200 Da to about 8,000,000 Da (also referred to as 8000 K or 8000 kDa), for example, about 200 Da to about 400 Da, about 400 Da to about 800 Da, about 800 Da to about 1600 Da, about 1600 Da to about 2500 Da, about 2500 Da to about 5000 Da, about 5000 Da to about 10 K, about 10 K to about 20 K, about 20 K to about 50 K, about 50 K to about 100 K, about 100 K to about 200 K, about 200 K to about 400 K, about 400 K to about 800 K, about 800 K to about 1000 K, about 1000 K to about 2000 K, about 2000 K to about 4000 K, about 4000 K to about 6000 K, or about 6000 K to about 8000 K. In some embodiments, the polymeric linker comprises up to 20% plasticizer, such as up to 18% plasticizer, up to 15% plasticizer, up to 12% plasticizer, up to 10% plasticizer, up to 8% plasticizer, up to 6% plasticizer, up to 4% plasticizer, up to 3% plasticizer, up to 2% plasticizer, or up to 1% plasticizer. In some embodiments, the polymeric linker comprises about 0.5% to about 20% plasticizer, such as about 0.5% to about 1%, about 1% to about 2%, about 2% to about 3%, about 3% to about 5%, about 5% to about 7%, about 7% to about 10%, about 10% to about 12%, about 12% to about 15% plasticizer, about 15% to about 18% plasticizer, or about 18% to about 20% plasticizer.

In some embodiments, the time-dependent polymeric linker includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The time-dependent polymeric linker may include the color-absorbing dye in an amount of up to about 5%, such as up to about 4%, up to about 3%, up to about 2%, up to about 1%, up to about 0.5%, up to about 0.3%, up to about 0.2%, or up to about 0.1%.

The time-dependent polymeric linker optionally includes one or more additional excipients. For example, the time-dependent polymeric linker may include a porogen, such as a sugar (e.g., lactose, sucrose, glucose, etc.), a salt (e.g., NaCl), sodium starch glycolate (SSG), or any other suitable substance. The porogen may quickly dissolve in the aqueous environment, which allows the aqueous solution to accelerate contact with the inner portions of the polymeric linker. Other excipients may include a flow aid, such as vitamin E succinate or silicified silicon dioxide (e.g., Cab-O-Sil), which may be included in the polymer blend for easier handling of the material prior to extrusion.

In one example of a time-dependent polymeric linker, the polymeric linker comprises about 75% to about 90% PLGA and about 10% to about 25% PLA (for example, about 85% PLGA and about 15% PLA). The PLA may be, for example, PLDL or PDL. The PLGA may be, for example, poly(D,L-lactic-co-glycolide) with a lactide monomer to glycolide monomer ratio of about 50:50 to about 75:25 (such as about 65:35) and/or have an inherent viscosity between about 0.1 dl/g and about 0.7 dl/g (such as about 0.3 dl/g to about 0.5 dl/g).

In another example of a time-dependent polymeric linker, the polymeric linker comprises about 40% to about 70% PLGA and about 30% to about 60% carrier polymer (for example, about 55% PLGA and about 45% PLA). The carrier polymer may be, for example, a TPU or a PCL. The PLGA may be, for example, (1) poly(D,L-lactic-co-glycolide) with a lactide monomer to glycolide monomer ratio of about 65:35 to about 95:5 (such as about 75:25) and/or have an inherent viscosity between about 0.1 dl/g and about 0.5 dl/g (such as about 0.15 dl/g to about 0.25 dl/g); (2) poly(D,L-lactic-co-glycolide) with a lactide monomer to glycolide monomer ratio of about 25:75 to about 75:25 (such as about 50:50) and/or have an inherent viscosity between about 0.5 dl/g and about 1.5 dl/g (such as about 0.8 dl/g to about 1.2 dl/g); or (3) poly(D,L-lactic-co-glycolide) with a lactide monomer to glycolide monomer ratio of about 65:35 to about 95:5 (such as about 75:25) and/or have an inherent viscosity between about 0.3 dl/g and about 1.2 dl/g (such as about 0.5 dl/g to about 0.9 dl/g).

In another example of a time-dependent polymeric linker, the polymeric linker comprises about 35% to about 65% (such as about 50%) carrier polymer, about 35% to about 65% (such as about 53%) PDLG, and about 2% polyethylene glycol (such as polyethylene glycol 100 K), and optionally further comprises about iron oxide (such as about 0.01% to about 0.25% iron oxide). The carrier polymer may be, for example, a TPU or a PCL.

In another example of a time-dependent polymeric linker, the polymeric linker comprises about 35% to about 45% (such as about 40%) carrier polymer (such as a TPU or a PCL), and about 55% to about 65% (such as about 60%) PLGA. The PLGA may be, for example acid-terminated PLGA

In another example of a time-dependent polymeric linker, the polymeric linker comprises about 40% to about 50% (such as about 45%) carrier polymer (such as a TPU or a PCL), about 48% to about 58% (such as about 53%) PLGA, and about 1% to about 3% (such as about 2%) polyethylene glycol (such as about polyethylene glycol 100 K). The PLGA may be, for example acid-terminated PLGA.

In another example of a time-dependent polymeric linker, the polymeric linker comprises about 40% to about 50% (such as about 45%) carrier polymer (such as a TPU or a PCL), about 48% to about 58% (such as about 53%) PLGA, wherein the PLGA comprises acid-terminated PLGA and ester-terminated PLAG at a ratio of about 4:1 to about 1:1, such as about 2:1. Optionally, the polymeric linker comprises about 1% to about 3% (such as about 2%) polyethylene glycol (such as about polyethylene glycol 100 K) and/or iron oxide (for example at about 0.01% to about 0.2%, such as about 0.05% to about 0.1%).

In another example of a time-dependent polymeric linker, the polymeric linker comprises about 45% to about 55% (such as about 50%) carrier polymer (such as a TPU or a PCL), and about 45% to about 55% (such as about 50%) PLGA. The PLGA may be, for example acid-terminated PLGA.

In another example of a time-dependent polymeric linker, the polymeric linker comprises about 45% to about 55% (such as about 50%) carrier polymer (such as a TPU or a PCL), about 40% to about 50% (such as about 45%) PLGA, and about 2% to about 7% (such as about 5%) polyethylene oxide (such as polyethylene glycol 100 K). The PLGA may be, for example acid-terminated PLGA.

In some embodiments, a time-dependent polymeric linker may comprise about 10 % or more, about 20 % or more, about 30 % or more, about 40 % or more, about 50 % or more, about 60 % or more, about 70 % or more, or about 80 % or more (by weight) PLGA. In some embodiments, a time-dependent polymeric linker may comprise about 90 % or less, about 80 % or less, about 70 % or less, about 60 % or less, about 50 % or less, about 40 % or less, about 30 % or less, or about 20 % or less (by weight) PLGA.

In some embodiments, a time-dependent polymeric linker may comprise 50 to 90 %, 60 to 80 %, 65 to 75 %, 68 to 72 %, or 70 % (by weight) PLGA. In some embodiments, a time-dependent polymeric linker may comprise 40 to 72 %, 45 to 67 %, 50 to 62 %, 54 to 58 %, or 56 % (by weight) PLGA. In some embodiments, a time-dependent polymeric linker may comprise 30 to 70 %, 40 to 60 %, 45 to 55 %, 48 to 52 %, or 50 % (by weight) PLGA. In some embodiments, a time-dependent polymeric linker may comprise 20 to 60 %, 30 to 50 %, 35 to 45 %, 38 to 42 %, or 40 % (by weight) PLGA.

In some embodiments, a time-dependent polymeric linker comprising PLGA may include a lactic acid to glycolic acid ratio of 5:95, 10:90, 15:85, 20:80, 25:75, 30:70, 35:65, 40:60, 45:55, 50:50, 55:45, 6-:40, 65:35, 70:30, 75:25, 80:20, 85:15, 90:10, or 95:5.

### Gastric Residence Time

The gastric residence time of the system is controlled by the degradation or weakening, or breakage, rate of the time-dependent polymeric linker in the gastric residence system. Faster degradation or weakening, or breakage of the time-dependent polymeric linker results in faster passage of the system from the stomach. The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Enteric Disintegrating Matrices (Enteric Linkers)

If the gastric residence system passes prematurely into the small intestine in an intact form, the system may be designed to break down much more rapidly to avoid intestinal obstruction. This is readily accomplished by using an enteric polymeric linker that includes an enteric polymer in addition to an additional linker polymer (such as a carrier polymer), which weakens or degrades within the intestinal environment. Enteric polymers are relatively resistant to the acidic pH levels encountered in the stomach, but dissolve rapidly at the higher pH levels found in the duodenum. Use of enteric polymeric linkers as safety elements protects against undesired passage of the intact gastric residence system into the small intestine. The use of enteric polymeric linker also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric polymeric linker.

Weakening or degradation of the enteric polymeric linker may be measured in references to a loss of the flexural modulus or breakage of the polymeric linker under a given condition (e.g., enteric conditions or gastric conditions). The enteric linkers weaken, degrade, or break in the intestinal environment relatively quickly, while retain much of their flexural modulus in the gastric environment. Stomach conditions may be simulated using an aqueous solution, such FaSSGF, at a pH of 1.6 and at 37° C., and intestinal conditions may be simulated using an aqueous solution, such as FaSSIF, at a pH 6.5 at 37° C. For example, in some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 12 hours at 37° C. In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 24 hours at 37° C. In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 2 days at 37° C. In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 3 days at 37° C. In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 4 days at 37° C. In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 5 days at 37° C.

In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 3 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 5 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 7 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 10 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 14 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 18 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 21 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 24 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 30 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 45 days at 37° C. In some embodiments, the enteric polymeric linker retains about 60% or more, about 70% or more, about 80% or more, about 90% or more, about 95% or more, or about 99% or more of its flexural modulus after incubation in an aqueous solution, such as FaSSGF, at pH 1.6 for 60 days at 37° C.

In some embodiments, the enteric polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 3 days at 37° C.; and the enteric polymeric linker retains about 60% or more, about 70% or more, about 80% or more, about 90% or more, about 95% or more, or about 99% or more of its flexural modulus or breaks after incubation in an aqueous solution, such as FaSSGF, at pH 1.6 for 7 days at 37° C.

The enteric polymeric linker weakens faster or to a greater extent in enteric conditions than in gastric conditions. For example, in some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 12 hours. I n some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 24 hours. In some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 36 hours. In some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 2 days. In some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 3 days. In some embodiments, the enteric polymeric linker loses its flexural modulus after incubation in an aqueous solution, such as FaSSIF, at pH 6.5 for 12 hours by more than about 5%, more than about 10%, more than about 15%, more than about 20%, more than about 25%, more than about 30%, more than about 35%, more than about 40%, more than about 50%, more than about 55%, more than about 60%, more than about 65%, more than about 70%, more than about 75%, more than about 80%, more than about 85%, more than about 90%, more than about 95%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95%, or any consecutive combination of such ranges, of the loss of its flexural modulus after incubation an aqueous solution, such as FaSSGF, at pH 1.6 for 4 days.

In some embodiments, the enteric polymeric linker has an initial flexural modulus of about 100 MPa to about 2500 MPa, such as about 100 MPa to about 250 MPa, about 250 MPa to about 500 MPa, about 500 mPa to about 750 MPa, about 750 MPa to about 1000 MPa, about 1000 MPa to about 1250 MPa, about 1250 MPa to about 1500 MPa, about 1500 MPa to about 2000 MPa, or about 2000 MPa to about 2500 MPa.

Exemplary enteric polymers that can be used in the invention are listed in the Enteric Polymer Table (Table 1), along with their dissolution pH. (See Mukherji, Gour and Clive G. Wilson, “Enteric Coating for Colonic Delivery,” Chapter 18 of Modified-Release Drug Delivery Technology (editors Michael J. Rathbone, Jonathan Hadgraft, Michael S. Roberts), Drugs and the Pharmaceutical Sciences Volume 126, New York: Marcel Dekker, 2002.) Preferably, enteric polymers that dissolve at a pH of no greater than about 5 or about 5.5 are used. Poly(methacrylic acid-co-ethyl acrylate) (sold under the trade name EUDRAGIT L 100-55; EUDRAGIT is a registered trademark of Evonik Röhm GmbH, Darmstadt, Germany) is a preferred enteric polymer. Another preferred enteric polymer is hydroxypropylmethylcellulose acetate succinate (hypromellose acetate succinate or HPMCAS; Ashland, Inc., Covington, Kentucky, USA), which has a tunable pH cutoff from about 5.5 to about 7.0. Cellulose acetate phthalate, cellulose acetate succinate, and hydroxypropyl methylcellulose phthalate are also suitable enteric polymers.

TABLE 1

Enteric Polymer Table

| Polymer | Dissolution pH |
|---|---|
| Cellulose acetate phthalate | 6.0-6.4 |
| Hydroxypropyl methylcellulose phthalate 50 | 4.8 |
| Hydroxypropyl methylcellulose phthalate 55 | 5.2 |
| Polyvinylacetate phthalate | 5.0 |
| Methacrylic acid-methyl methacrylate copolymer (1:1) | 6.0 |
| Methacrylic acid-methyl methacrylate copolymer (2:1) | 6.5-7.5 |
| Methacrylic acid-ethyl acrylate copolymer (2:1) | 5.5 |
| Shellac | 7.0 |
| Hydroxypropyl methylcellulose acetate succinate | 7.0 |
| Poly (methyl vinyl ether/maleic acid) monoethyl ester | 4.5-5.0 |
| Poly (methyl vinyl ether/maleic acid) n-butyl ester | 5.4 |

The amount of enteric polymer included in the enteric polymeric linker can be selected based on the desired linker weakening or degradation profile. For example, the polymeric linker may include about 1% to about 99% enteric polymer, such as about 1% to about 5% enteric polymer, about 5% to about 10% enteric polymer, about 10% to about 20% enteric polymer, about 20% to about 30% enteric polymer, about 30% to about 40% enteric polymer, about 40% to about 50% enteric polymer, about 50% to about 60% enteric polymer, about 60% to about 70% enteric polymer, about 70% to about 80% enteric polymer, about 80% to about 90% enteric polymer, or about 90% to about 99% enteric polymer. In some embodiments, the enteric polymeric linker comprises less than 20% enteric polymer. In some embodiments, the enteric polymeric linker comprises less than 15% enteric polymer. In some embodiments, the enteric clinker comprises less than 10% enteric polymer. In some embodiments, the enteric linker comprises more than 80% enteric polymer. In some embodiments, the enteric linker comprises more than 85% enteric polymer. In some embodiments, the enteric linker comprises more than 90% enteric polymer. In some embodiments, the enteric linker comprises about 20% to about 80% enteric polymer.

In some embodiments, the enteric polymer comprises hydroxypropyl methylcellulose acetate succinate (HPMCAS). For example, in some embodiments, the polymeric linker includes about 1% to about 99% HPMCAS, such as about 1% to about 5% HPMCAS, about 5% to about 10% HPMCAS, about 10% to about 20% HPMCAS, about 20% to about 30% HPMCAS, about 30% to about 40% HPMCAS, about 40% to about 50% HPMCAS, about 50% to about 60% HPMCAS, about 60% to about 70% HPMCAS, about 70% to about 80% HPMCAS, about 80% to about 90% HPMCAS, or about 90% to about 99% HPMCAS. In some embodiments, the enteric polymeric linker comprises less than 20% HPMCAS. In some embodiments, the enteric polymeric linker comprises less than 15% HPMCAS. In some embodiments, the enteric clinker comprises less than 10% HPMCAS. In some embodiments, the enteric linker comprises more than 80% HPMCAS. In some embodiments, the enteric linker comprises more than 85% HPMCAS. In some embodiments, the enteric linker comprises more than 90% HPMCAS. In some embodiments, the enteric linker comprises about 20% to about 80% HPMCAS.

The enteric polymer is combined with one or more additional polymers (such as one or more carrier polymers) in the enteric linker, preferably in a homogenous mixture. For example, the enteric polymer and the additional linker polymer may be homogenously blended together before the mixture is extruded, and the extruded material being cut to a desired size for the polymeric linker. In some embodiments, the one or more additional linker polymers are miscible with the enteric polymer. The one or more additional linker polymers may be a non-degradable polymer (that is, not degradable or in the gastric or enteric environment, or an aqueous solution of pH 1.6 (representing the gastric environment) or pH 6.5 (representing the enteric environment).

Bonding of the polymeric linker to a directly adjacent member may be improved if at least one polymer is common to both the adjacent member and the enteric polymeric linker. That is, one of the one or more additional linker polymers in the enteric linker may be the same (or the same polymer type) as at least one polymer in a directly adjacent component (or, optionally, both directly adjacent components) of the gastric residence system. For example, if the enteric polymeric linker is bonded directly to a structural member comprising a carrier polymer, in some embodiments the one or more additional linker polymers also includes the carrier polymer (in addition to the PLGA in the time-dependent polymeric linker) at the same or different concentration. Exemplary carrier polymers include, but are not limited to, polylactic acid (PLA), polycaprolactone (PCL), and a thermoplastic polyurethane (TPU), among others described herein.

In some embodiments, the one or more additional linker polymers in the enteric polymeric linker is PLA, for example a PLA as described herein in reference to carrier polymers. In some embodiments, the enteric polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) PLA. In some embodiments, the enteric polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) PLA. In some embodiments, the enteric polymeric linker includes about 0.1% to about 10% PLA, about 10% to about 20% PLA, about 20% to about 30% PLA, about 30% to about 40% PLA about 40% to about 50% PLA, about 50% to about 60% PLA, about 60% to about 70% PLA, about 70% to about 80% PLA, about 80% to about 90% PLA, or about 90% to about 99.9% PLA. In some embodiments, the enteric polymeric linker includes about 30% PLA or less. In some embodiments, the enteric polymeric linker includes about 70% PLA or more. In some embodiments, the enteric polymeric liker includes about 30% to about 70% PLA. The enteric polymer (such as HPMCAS) is further included with the PLA, and can make up to the balance of the enteric polymeric linker, although additional agents (such as a plasticizer, a coloring agent, or other agent may be further included).

In some embodiments, the one or more additional linker polymers in the enteric linker comprises a PCL. The enteric polymeric linker may be directly joined or bonded to another member of the gastric residence system (such as the structural member comprising the drug and the carrier polymer, a coupling member, the time-dependent polymeric linker, or a central structural member), which may also include a PCL, which may be the same PCL in the enteric polymeric linker or a different PCL as the one in the enteric polymeric linker, and which may be at the same concentration or a different concentration. A different PCL in the enteric polymeric linker and the other member directly joined or bonded to the enteric linker may differ, for example, in the weight-average molecular weight of the PCL, the inherent viscosity of the PCL, or the proportions of PCL (for example, when a blend of two or more PCL polymers are used).

In some embodiments, the enteric polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) PCL. In some embodiments, the enteric polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) PCL. In some embodiments, the enteric polymeric linker includes about 0.1% to about 10% PCL, about 10% to about 20% PCL, about 20% to about 30% PCL, about 30% to about 40% PCL about 40% to about 50% PCL, about 50% to about 60% PCL, about 60% to about 70% PCL, about 70% to about 80% PCL, about 80% to about 90% PCL, or about 90% to about 99.9% PCL. In some embodiments, the enteric polymeric linker includes about 30% PLA or less. In some embodiments, the enteric polymer linker includes about 70% PLA or more. In some embodiments, the enteric polymeric liker includes about 30% to about 70% PCL. The enteric polymer (such as HPMCAS) is further included with the PCL, and can make up to the balance of the enteric polymeric linker, although additional agents (such as a plasticizer, a coloring agent, or other agent may be further included).

In some embodiments, the one or more additional linker polymers in the enteric polymeric linker comprises a TPU. The enteric polymeric linker may be directly joined or bonded to another member of the gastric residence system (such as the structural member comprising the drug and the carrier polymer, a coupling member, the time-dependent polymeric linker, or a central structural member), which may also include a TPU, which may be the same TPU in the enteric polymeric linker or a different TPU as the one in the enteric polymeric linker, and which may be at the same concentration or a different concentration. A different TPU in the enteric polymeric linker and the other member directly joined or bonded to the enteric linker may differ, for example, in the weight-average molecular weight of the TPU, the inherent viscosity of the TPU, or the proportions of TPU (for example, when a blend of two or more TPU polymers are used). Suitable commercially-available TPU polymers may include Pathway™ TPU polymers (The Lubrizol Corporation), Tecoflex™ (The Lubrizol Corporation), Tecophilic™ (The Lubrizol Corporation), Carbothane™ (The Lubrizol Corporation), Texin® (Covestro), and NEUSoft™ (PolyOne). Additionally, suitable types of TPU polymers for the polymeric linker can include aliphatic TPUs, aliphatic polyether TPUs, aromatic TPUs, polycarbonate polyurethanes, and the like.

In some embodiments, the enteric polymeric linker includes about 99% or less, about 98% or less, about 95% or less, about 90% or less, about 85% or less, about 80% or less, about 75% or less, about 70% or less, about 65% or less, about 60% or less, about 55% or less, about 50% or less, about 40% or less, about 30% or less, about 20% or less, or about 10% or less (by weight) TPU. In some embodiments, the enteric polymeric linker includes about 99% or more, about 98% or more, about 95% or more, about 90% or more, about 85% or more, about 80% or more, about 75% or more, about 70% or more, about 65% or more, about 60% or more, about 55% or more, about 50% or more, about 40% or more, about 30% or more, about 20% or more, or about 10% or more (by weight) TPU. In some embodiments, the enteric polymeric linker includes about 0.1% to about 10% TPU, about 10% to about 20% TPU, about 20% to about 30% TPU, about 30% to about 40% TPU about 40% to about 50% TPU, about 50% to about 60% TPU, about 60% to about 70% TPU, about 70% to about 80% TPU, about 80% to about 90% TPU, or about 90% to about 99.9% TPU. In some embodiments, the enteric polymeric linker includes about 30% TPU or less. In some embodiments, the enteric polymer linker includes about 70% TPU or more. In some embodiments, the enteric polymeric liker includes about 30% to about 70% TPU. In some embodiments, the enteric polymeric liker includes about 30% to about 70% PLA. The enteric polymer (such as HMPCAS) may be further included with the TPU, and can make up to the balance of the enteric polymeric linker, although additional agents (such as a plasticizer, a color-absorbing dye, or other agent may be further included).

In some embodiments, an enteric polymeric linker may include 1 to 40 %, 5 to 35 %, 10 to 30 %, 15 to 25 %, 18 to 22 %, or 20 % (by weight) TPU.

The enteric polymeric linker may further include one or more plasticizers, which can aid in cutting an extruded polymeric linker material to a desired size and aid in bonding or attaching the enteric polymeric linker to other components of the gastric residence system. Exemplary plasticizers include, but are not limited to, propylene glycol, polyethylene glycol (PEG), triethyl butyl citrate (TBC), dibutyl sebacate (DBS), triacetin, triethyl citrate (TEC), a poloxamer (e.g., Poloxamer 407, or “P407”), or D-α-tocopheryl polyethylene glycol succinate. In some embodiments, the molecular weight of the polyethylene glycol is about 200 Da to about 8,000,000 Da (also referred to as 8000 K or 8000 kDa), for example, about 200 Da to about 400 Da, about 400 Da to about 800 Da, about 800 Da to about 1600 Da, about 1600 Da to about 2500 Da, about 2500 Da to about 5000 Da, about 5000 Da to about 10 K, about 10 K to about 20 K, about 20 K to about 50 K, about 50 K to about 100 K, about 100 K to about 200 K, about 200 K to about 400 K, about 400 K to about 800 K, about 800 K to about 1000 K, about 1000 K to about 2000 K, about 2000 K to about 4000 K, about 4000 K to about 6000 K, or about 6000 K to about 8000 K. In some embodiments, the polymeric linker comprises up to 20% plasticizer, such as up to 18% plasticizer, up to 15% plasticizer, up to 12% plasticizer, up to 10% plasticizer, up to 8% plasticizer, up to 6% plasticizer, up to 4% plasticizer, up to 3% plasticizer, up to 2% plasticizer, or up to 1% plasticizer. In some embodiments, the polymeric linker comprises about 0.5% to about 15% plasticizer, such as about 0.5% to about 1%, about 1% to about 2%, about 2% to about 3%, about 3% to about 5%, about 5% to about 7%, about 7% to about 10%, about 10% to about 12%, about 12% to about 15% plasticizer, about 15% to about 18% plasticizer, or about 18% to about 20% plasticizer.

In some embodiments, the enteric polymeric linker includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The enteric polymeric linker may include the color-absorbing dye in an amount of up to about 5%, such as up to about 4%, up to about 3%, up to about 2%, up to about 1%, up to about 0.5%, up to about 0.3%, up to about 0.2%, or up to about 0.1%.

The enteric polymeric linker optionally includes one or more additional excipients. For example, the enteric polymeric linker may include a porogen, such as a sugar (e.g., lactose, sucrose, glucose, etc.), a salt (e.g., NaCl), sodium starch glycolate (SSG), or any other suitable substance. The porogen may quickly dissolve in the aqueous environment, which allows the aqueous solution to accelerate contact with the inner portions of the polymeric linker. Other excipients may include a flow aid, such as vitamin E succinate or silicified silicon dioxide (e.g., Cab-O-Sil), which may be included in the polymer blend for easier handling of the material prior to extrusion.

In some embodiments, the enteric polymeric linker comprises about 30% to about 80% HPMCAS and about 20% to about 70% carrier polymer (such as a TPU or a PCL). Optionally, the enteric polymeric linker further comprises propylene glycol (for example, about 10% to about 14% propylene glycol).

In some embodiments, the enteric polymeric linker comprises about 55% to about 65% (such as about 60%) HPMCAS and about 35% to about 45% (such as about 40%) carrier polymer (such as a TPU or a PCL).

In some embodiments, the enteric polymeric linker comprises about 35% to about 45% (such as about 40%) HPMCAS, about 45% to about 55% (such as about 50%) carrier polymer (such as a TPU or a PCL), and propylene glycol (for example, about 8% to about 12% propylene glycol, such as about 10% propylene glycol).

In some embodiments, the enteric polymeric linker comprises about 43% to about 53% (such as about 48%) HPMCAS, about 35% to about 45% (such as about 40%) carrier polymer (such as a TPU or a PCL), and propylene glycol (for example, about 10% to about 14% propylene glycol, such as about 12% propylene glycol).

In some embodiments, the enteric polymeric linker comprises about 51% to about 61% (such as about 56%) HPMCAS, about 25% to about 35% (such as about 30%) carrier polymer (such as a TPU or a PCL), and propylene glycol (for example, about 12% to about 16% propylene glycol, such as about 14% propylene glycol).

In some embodiments, the enteric polymeric linker comprises about 52% to about 62% (such as about 57%) HPMCAS, about 35% to about 45% (such as about 40%) carrier polymer (such as a TPU or a PCL), and propylene glycol (for example, about 1% to about 5% propylene glycol, such as about 3% propylene glycol).

In some embodiments, the enteric polymeric linker comprises about 49% to about 59% (such as about 54%) HPMCAS, about 35% to about 45% (such as about 40%) carrier polymer (such as a TPU or a PCL), and propylene glycol (for example, about 4% to about 8% propylene glycol, such as about 6% propylene glycol).

In some embodiments, the enteric polymeric linker comprises about 45% to about 55% (such as about 50%) HPMCAS and about 45% to about 55% (such as about 55%) carrier polymer (such as a TPU or a PCL). Optionally, the enteric polymeric linker further comprises iron oxide, for example about 0.01% to about 0.2 % (such as about 0.05% to about 0.1%) iron oxide.

In some embodiments, the enteric polymeric linker comprises about 55% to about 65% (such as about 60%) HPMCAS and about 35% to about 45% (such as about 40%) carrier polymer (such as a TPU or a PCL). Optionally, the enteric polymeric linker further comprises iron oxide, for example about 0.01% to about 0.2 % (such as about 0.05% to about 0.1%) iron oxide.

In some embodiments, the enteric polymeric linker comprises about 53% to about 63% (such as about 58%) HPMCAS and about 33% to about 43% (such as about 38%) carrier polymer (such as a TPU or a PCL), and about 2% to about 6% (such as about 4%) polyethylene glycol (such as polyethylene glycol 100 K). Optionally, the enteric polymeric linker further comprises iron oxide, for example about 0.01% to about 0.2 % (such as about 0.05% to about 0.1%) iron oxide.

In some embodiments, the enteric polymeric linker comprises about 31% to about 41% (such as about 36%) HPMCAS and about 31% to about 41% (such as about 36%) carrier polymer (such as a TPU or a PCL), and about 23% to about 33% (such as about 28%) TEC. Optionally, the enteric polymeric linker further comprises iron oxide, for example about 0.01% to about 0.2 % (such as about 0.05% to about 0.1%) iron oxide.

In some embodiments, the enteric polymeric linker comprises about 59% to about 69% (such as about 64%) HPMCAS and about 29% to about 39% (such as about 34%) carrier polymer (such as a TPU or a PCL), and about 1% to about 3% (such as about 2%) poloxamer (such as P407). Optionally, the enteric polymeric linker further comprises iron oxide, for example about 0.01% to about 0.2 % (such as about 0.05% to about 0.1%) iron oxide.

In some embodiments, the enteric polymeric linker comprises about 59% to about 69% (such as about 64%) HPMCAS and about 29% to about 39% (such as about 34%) carrier polymer (such as a TPU or a PCL), and about 1% to about 3% (such as about 2%) polyethylene glycol (such as polyethylene glycol 100 K). Optionally, the enteric polymeric linker further comprises iron oxide, for example about 0.01% to about 0.2 % (such as about 0.05% to about 0.1%) iron oxide.

In some embodiments, the enteric polymeric linker comprises about 65% to about 75% (such as about 70%) HPMCAS and about 25% to about 35% (such as about 30%) carrier polymer (such as a TPU or a PCL). Optionally, the enteric polymeric linker further comprises iron oxide, for example about 0.01% to about 0.2 % (such as about 0.05% to about 0.1%) iron oxide.

In some embodiments, the enteric polymeric linker comprises about 79% to about 89% (such as about 84%) HPMCAS and about 9% to about 19% (such as about 14%) carrier polymer (such as a TPU or a PCL), and about 1% to about 3% (such as about 2%) polyethylene glycol (such as polyethylene glycol 100 K). Optionally, the enteric polymeric linker further comprises iron oxide, for example about 0.01% to about 0.2 % (such as about 0.05% to about 0.1%) iron oxide.

In some embodiments, the enteric polymeric linker comprises about 70% to about 80% (such as about 75%) HPMCAS and about 10% to about 20% (such as about 15%) carrier polymer (such as a TPU or a PCL), and about 5% to about 15% (such as about 10%) TEC. Optionally, the enteric polymeric linker further comprises iron oxide, for example about 0.01% to about 0.2 % (such as about 0.05% to about 0.1%) iron oxide.

### Dual Time-Dependent and Enteric Linkers

In some embodiments, the gastric residence system includes a polymeric linker that includes both time-dependent and enteric functionalities. That is, the dual time-dependent and enteric polymeric linker weakens or degrades in both the gastric and intestinal environments, although weakening and degradation of the linker is faster in the intestinal environment than the gastric environment. This type of linker may be obtained, for example, by including a mixture of a pH-independent degradable polymer, such as PLGA, with an enteric polymer, such as HPMCAS.

For example, in some embodiments, the polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 12 hours at 37° C., retains about 60% or more, about 70% or more, about 80% or more, about 90% or more, about 95% or more, or about 99% or more of its flexural modulus after incubation in an aqueous solution, such as FaSSGF, at pH 1.6 for 12 hours at 37° C., and loses about 60% or more, about 70% or more, about 80% or more, about 90% or more, about 95% or more, or about 99% or more of its flexural modulus after incubation in an aqueous solution, such as FaSSGF, at pH 1.6 for 7 days at 37° C. In some embodiments, the polymeric linker loses about 60% or more, about 70% or more, about 80% or more, about 90% or more, about 95% or more, or about 99% or more of its flexural modulus or breaks after incubation in an aqueous solution, such as FaSSIF, at pH 6.5 for 24 hours at 37° C., retains about 60% or more, about 70% or more, about 80% or more, about 90% or more, about 95% or more, or about 99% or more of its flexural modulus after incubation in an aqueous solution, such as FaSSGF, at pH 1.6 for 24 hours at 37° C., and loses about 60% or more, about 70% or more, about 80% or more, about 90% or more, about 95% or more, or about 99% or more of its flexural modulus after incubation in an aqueous solution, such as FaSSGF, at pH 1.6 for 7 days at 37° C.

In some embodiments, the dual time-dependent polymeric linker has an initial flexural modulus of about 100 MPa to about 2500 MPa, such as about 100 MPa to about 2500 MPa, such as about 100 MPa to about 250 MPa, about 250 MPa to about 500 MPa, about 500 mPa to about 750 MPa, about 750 MPa to about 1000 MPa, about 1000 MPa to about 1250 MPa, about 1250 MPa to about 1500 MPa, about 1500 MPa to about 2000 MPa, or about 2000 MPa to about 2500 MPa.

In some embodiments, the dual time-dependent and enteric polymeric linker comprises PLGA. Examples of PLGA that may be included in the dual time-dependent and enteric polymeric linker is discussed above in reference to the time-dependent polymeric linker. In some embodiments, the dual time-dependent and enteric polymeric linker includes about 60% or less, about 55% or less, about 50% or less, about 40% or less, about 30% or less, about 20% or less, or about 10% or less (by weight) PLGA. In some embodiments, the dual time-dependent and enteric polymeric linker includes about 50% or more, about 40% or more, about 30% or more, about 20% or more, or about 10% or more (by weight) PLGA. In some embodiments, the dual time-dependent and enteric polymeric linker includes about 5% to about 60% PLGA, such as about 5% to about 10% PLGA, about 10% to about 20% PLGA, about 20% to about 30% PLGA, about 30% to about 40% PLGA, about 40% to about 50% PLGA, or about 50% to about 60% PLGA.

In some embodiments, the dual time-dependent and enteric polymeric linker includes about 60% or less, about 55% or less, about 50% or less, about 40% or less, about 30% or less, about 20% or less, or about 10% or less (by weight) enteric polymer, such as HPMCAS. In some embodiments, the dual time-dependent and enteric polymeric linker includes about 50% or more, about 40% or more, about 30% or more, about 20% or more, or about 10% or more (by weight) PLGA. In some embodiments, the dual time-dependent and enteric polymeric linker includes about 5% to about 60% enteric polymer, such as HPMCAS, such as about 5% to about 10%, about 10% to about 20%, about 20% to about 30%, about 30% to about 40%, about 40% to about 50%, or about 50% to about 60% enteric polymer, such as HPMCAS.

In some embodiments, the dual time-dependent and enteric polymeric linker comprise about 40% to about 80% HPMCAS and about 20% to about 60% PLGA. Optionally, the polymeric linker further comprises a carrier polymer (such as PLA, TPU, or PCL), for example at about 5% to about 40%.

### Structural Members

The gastric residence system includes one or more structural members (e.g., “arms”) attached to a second member (e.g., a central member, which may be an elastomeric member) through one or more polymeric linkers (e.g., a time-dependent polymeric linker, an enteric polymeric linker, both a time-dependent polymeric linker and an enteric polymeric linker, or a dual time-dependent and enteric polymeric linker). The structural members include a carrier polymer and an agent (such a as a drug). In some embodiments, the structural member are segmented into a plurality of segments. For example, the structural member may include one or more active segments (e.g., a segment containing an agent) and one or more inactive segments. An embodiment with two or more active segments may have identical or different active segments (for example, a first active segment containing a first agent, and a second active segment containing a second agent). In some embodiments, active segments and inactive segments include a common carrier polymer or a common type of carrier polymer.

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges. The cross-section of the segments and arms may match the cross-section of the one or more polymeric linkers used to attach the arms to the second (e.g., central) member.

### Carrier PolymerAgent Segments (Drug-Eluting Segments)

The carrier polymer-agent segments, or drug-eluting segments, release an agent in a controlled manner during the period that the gastric residence system resides in the stomach. The carrier polymer is blended with the agent, and formed into segments which are then assembled with the other components described herein to manufacture the gastric residence system. This mixture can be formed into the desired shape or shapes for use as carrier polymer-agent components in the systems. After the agent is blended into the carrier polymer to form the carrier polymer-drug mixture, the drug or drug salt is distributed or dispersed throughout the blended mixture. If excipients, anti-oxidants, or other ingredients are included in the carrier polymer-drug blend, they will also be distributed or dispersed throughout the blended mixture.

Examples of carrier polymers that may be included in the carrier polymer-agent segments are described in further detail herein.

Agents which can be administered to or via the gastrointestinal tract can be used in the gastric residence systems of the invention. The agent is blended with the carrier polymer, and any other excipients or other additives to the carrier polymer, and formed into a segment for use in a gastric residence system. Agents include, but are not limited to, drugs, pro-drugs, biologics, and any other substance which can be administered to produce a beneficial effect on an illness or injury. Agents that can be used in the gastric residence systems of the invention include statins, such as rosuvastatin; nonsteroidal anti-inflammatory drugs (NSAIDs) such as meloxicam; selective serotonin reuptake inhibitors (SSRIs) such as escitalopram and citalopram; blood thinners, such as clopidogrel; steroids, such as prednisone; antipsychotics, such as aripiprazole and risperidone; analgesics, such as buprenorphine; opioid antagonists, such as naloxone; antiasthmatics such as montelukast; anti-dementia drugs, such as memantine; cardiac glycosides such as digoxin; alpha blockers such as tamsulosin; cholesterol absorption inhibitors such as ezetimibe; anti-gout treatments, such as colchicine; antihistamines, such as loratadine and cetirizine, opioids, such as loperamide; proton-pump inhibitors, such as omeprazole;, antiviral agents, such as entecavir; antibiotics, such as doxycycline, ciprofloxacin, and azithromycin; antimalarial agents; levothyroxine; substance abuse treatments, such as methadone and varenicline; contraceptives; stimulants, such as caffeine; and nutrients such as folic acid, calcium, iodine, iron, zinc, thiamine, niacin, vitamin C, vitamin D, biotin, plant extracts, phytohormones, and other vitamins or minerals. Biologics that can be used as agents in the gastric residence systems of the invention include proteins, polypeptides, polynucleotides, and hormones. Exemplary classes of agents include, but are not limited to, analgesics; anti-analgesics; anti-inflammatory drugs; antipyretics; antidepressants; antiepileptics; antipsychotic agents; neuroprotective agents; anti-proliferatives, such as anti-cancer agents; antihistamines; antimigraine drugs; hormones; prostaglandins; antimicrobials, such as antibiotics, antifungals, antivirals, and antiparasitics; anti-muscarinics; anxiolytics; bacteriostatics; immunosuppressant agents; sedatives; hypnotics; antipsychotics; bronchodilators; anti-asthma drugs; cardiovascular drugs; anesthetics; anticoagulants; enzyme inhibitors; steroidal agents; steroidal or non-steroidal anti-inflammatory agents; corticosteroids; dopaminergics; electrolytes; gastro-intestinal drugs; muscle relaxants; nutritional agents; vitamins; parasympathomimetics; stimulants; anorectics; anti-narcoleptics; and antimalarial drugs, such as quinine, lumefantrine, chloroquine, amodiaquine, pyrimethamine, proguanil, chlorproguanil-dapsone, sulfonamides (such as sulfadoxine and sulfamethoxypyridazine), mefloquine, atovaquone, primaquine, halofantrine, doxycycline, clindamycin, artemisinin, and artemisinin derivatives (such as artemether, dihydroartemisinin, arteether and artesunate). The term “agent” includes salts, solvates, polymorphs, and co-crystals of the aforementioned substances. In certain embodiments, the agent is selected from the group consisting of cetirizine, rosuvastatin, escitalopram, citalopram, risperidone, olanzapine, donepezil, and ivermectin. In another embodiment, the agent is one that is used to treat a neuropsychiatric disorder, such as an anti-psychotic agent or an anti-dementia drug such as memantine.

In some embodiments, the agent or salt thereof (for example, a drug) makes up about 10% to about 40% by weight of the arm or segment, and thus the carrier polymer and any other components of the arm or segment blended into the carrier polymer together make up the remainder of the weight of the arm or segment. In some embodiments, the agent or salt thereof makes up about 10% to about 35%, about 10% to about 30%, about 10% to about 25%, about 10% to about 20%, about 10% to about 15%, about 15% to about 40%, about 20% to about 40%, about 25% to about 40%, about 30% to about 40%, about 35% to about 40%, about 15% to about 35%, about 20% to about 35%, or about 25% to about 40% by weight of the arm or segment.

Other excipients can be added to the carrier polymers to modulate the release of agent. Such excipients can be added in amounts from about 1% to 15%, preferably from about 5% to 10%, more preferably about 5% or about 10%. Examples of such excipients include Poloxamer 407 (available as Kolliphor P407, Sigma Cat # 62035), poly(ethylene glycol)-block-poly(propylene glycol)-block-poly(ethylene glycol), CAS No. 9003-11-6; H—(OCH2CH2)x—(O—CH(CH3)CH2)y—(OCH2CH2)z—OH where x and z are about 101 and y is about 56); Pluronic P407; Eudragit E, Eudragit EPO (available from Evonik); hypromellose (available from Sigma, Cat # H3785), Kolliphor RH40 (available from Sigma, Cat # 07076), polyvinyl caprolactam, polyvinyl acetate (PVAc), polyvinylpyrrolidone (PVP), polyvinyl alcohol (PVA), polyethylene glycol (PEG), and Soluplus (available from BASF; a copolymer of polyvinyl caprolactam, polyvinyl acetate, and polyethylene glycol). Preferred soluble excipients include Eudragit E, polyethylene glycol (PEG), polyvinylpyrrolidone (PVP), polyvinyl acetate (PVAc), and polyvinyl alcohol (PVA). Preferred insoluble excipients include Eudragit RS and Eudragit RL. Preferred insoluble, swellable excipients include crospovidone, croscarmellose, hypromellose acetate succinate (HPMCAS), and carbopol. EUDRAGIT RS and EUDRAGIT RL are registered trademarks of Evonik (Darmstadt, Germany) for copolymers of ethyl acrylate, methyl methacrylate and methacrylic acid ester with quaternary ammonium groups (trimethylammonioethyl methacrylate chloride), having a molar ratio of ethyl acrylate, methyl methacrylate and trimethylammonioethyl methacrylate of about 1:2:0.2 in Eudragit® RL and about 1:2:0.1 in Eudragit® RS. Preferred insoluble, swellable excipients include crospovidone, croscarmellose, hypromellose acetate succinate (HPMCAS), carbopol, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%).

### Inactive Segments

The arms of the gastric residence system may include one or more inactive segments that are free or substantially free of an agent. “Substantially free” of the agent refers to the absence of the agent or the presence of agent in trace amounts that may be present due to diffusion from or bonding to an adjacent member, or normal handling, packaging, or storage of the gastric residence system, and without including the agent with the polymer or other material used to form the inactive segment.

The inactive segment may include a carrier polymer, which may be the same carrier polymer (or type of carrier polymer) or a different carrier polymer (or a different type of carrier polymer) used in the active carrier polymer-agent segment or in one or more of the polymeric linkers described herein. Exemplary carrier polymers are further described herein. In some embodiments, the carrier polymer included in the inactive segment is a thermoplastic polyurethane (TPU). In some embodiments, the carrier polymer included in the inactive segment is a polycaprolactone (PCL).

### Carrier Polymers

Preferably, carrier polymers have the following characteristics. They should be thermoplastic, to allow extrusion using hot melt extrusion or 3D printing techniques. They should also have a high enough melt strength and viscosity to enable extrusion into the required geometry. They should have low melting temperatures (for example, less than about 120° C.), to avoid exposing agents or drugs to high temperatures during manufacture. They should have sufficient mechanical strength (Young’s modulus, compression strength, tensile strength) to avoid breaking in the stomach during the desired residence period. They should be capable of forming stable blends with agents, therapeutic agents, drugs, excipients, dispersants, and other additives.

Exemplary carrier polymers suitable for use in this invention include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof. Polycaprolactone (PCL) is a preferred carrier polymer. In another embodiment, polydioxanone is used as the carrier polymer. In any of the embodiments of the gastric residence system, the carrier polymer used in the gastric residence system can comprise polycaprolactone, such as linear polycaprolactone with a number-average molecular weight (Mn) range between about 60 kiloDalton (kDa) to about 100 kDa; 75 kDa to 85 kDa; or about 80 kDa; or between about 45 kDa to about 55 kDa; or between about 50 kDa to about 110,000 kDa, or between about 80 kDa to about 110,000 kDa.

In some embodiments, the carrier polymer comprises polylactic acid (PLA). The PLA may comprise, consist essentially of, or consist of a poly(L-lactide), a poly(D-lactide), a poly (D,L-lactide), or combinations thereof or copolymers thereof (such as an L-lactide/D,L-lactide copolymer (PLDL), or an L-lactide/D-lactide copolymer (PLD)). In some embodiments, the inherent viscosity (as measured in CHCl₃ at 25° C.) of the PLA is between about 0.1 dl/g to about 6.5 dl/g, such as about 0.1 dl/g to about 0.15 dl/g, about 0.15 dl/g to about 0.25 dl/g, about 0.25 dl/g to about 0.5 dl/g, about 0.5 dl/g to about 0.75 dl/g, about 0.75 dl/g to about 1.0 dl/g, about 1.0 dl/g to about 1.25 dl/g, about 1.25 dl/g to about 1.5 dl/g, about 1.5 dl/g to about 2.0 dl/g, about 2.0 to about 2.5 dl/g, about 2.5 dl/g to about 3.0 dl/g, about 3.0 dl/g to about 3.5 dl/g, about 3.5 dl/g to about 4.0 dl/g, about 4.0 dl/g to about 4.5 dl/g, about 4.5 dl/g to about 5.0 dl/g, about 5.0 dl/g to about 5.5 dl/g, about 5.5 dl/g to about 6.0 dl/g or about 6.0 dl/g to about 6.5 dl/g. In some embodiments, the ratio of L-lactide monomers to D,L-lactide monomers in the PLDL copolymer ranges from about 5:95 to about 95:5, such as about 5:95 to about 10:90, about 10:90 to about 20:80, about 20:80 to about 35:65, about 35:65 to about 50:50, about 50:50 to about 65:35, about 65:35 to about 80:20, about 80:20 to about 90:10, or about 90:10 to about 95:5. In some embodiments, the ratio of L-lactide monomers to D-lactide monomers in the PLD copolymer ranges from about 5:95 to about 95:5, such as about 5:95 to about 10:90, about 10:90 to about 20:80, about 20:80 to about 35:65, about 35:65 to about 50:50, about 50:50 to about 65:35, about 65:35 to about 80:20, about 80:20 to about 90:10, or about 90:10 to about 95:5. In some embodiments, the PLA comprises a poly(L-lactide) with an inherent viscosity between 0.9 dl/g and 1.2 dl/g (measured in CHCl₃ at 25° C.) (such as the polymer sold under the tradename Purasorb® PL10). In some embodiments, the PLA comprises a poly(L-lactide) with an inherent viscosity between 1.5 dl/g and 2.0 dl/g (measured in CHCl₃ at 25° C.) (such as the polymer sold under the tradename Purasorb® PL18). In some embodiments, the PLA comprises a poly(L-lactide) with an inherent viscosity between 2.0 dl/g and 2.7 dl/g (measured in CHCl₃ at 25° C.) (such as the polymer sold under the tradename Purasorb® PL24). In some embodiments, the PLA comprises a poly(L-lactide) with an inherent viscosity between 2.7 dl/g and 3.6 dl/g (measured in CHCl₃ at 25° C.) (such as the polymer sold under the tradename Purasorb® PL32). In some embodiments, the PLA comprises a poly(L-lactide) with an inherent viscosity between 3.2 dl/g and 4.3 dl/g (measured in CHCl₃ at 25° C.) (such as the polymer sold under the tradename Purasorb® PL38). In some embodiments, the PLA comprises a poly(L-lactide) with an inherent viscosity between 4.3 dl/g and 5.5 dl/g (measured in CHCl₃ at 25° C.) (such as the polymer sold under the tradename Purasorb® PL49). In some embodiments, the PLA comprises a poly(L-lactide) with an inherent viscosity between 5.5 dl/g and 7.5 dl/g (measured in CHCl₃ at 25° C.) (such as the polymer sold under the tradename Purasorb® PL65). In some embodiments, the PLA comprises a poly(D-lactide) with an inherent viscosity between 2.0 dl/g and 2.8 dl/g (measured in CHCl₃ at 25° C.) (such as the polymer sold under the tradename Purasorb® PD24). In some embodiments, the PLA comprises a poly(D-lactide) with an inherent viscosity between 3.2 dl/g and 4.3 dl/g (measured in CHCl₃ at 25° C.) (such as the polymer sold under the tradename Purasorb® PD38). In some embodiments, the PLA comprises a poly(D,L-lactide) with an inherent viscosity between 0.4 dl/g and 0.6 dl/g (measured in CHCl₃ at 25° C.) (such as the polymer sold under the tradename Purasorb® PDL05). In some embodiments, the PLA comprises a poly(D,L-lactide) with an inherent viscosity between 1.6 dl/g and 2.4 dl/g (measured in CHCl₃ at 25° C.) (such as the polymer sold under the tradename Purasorb® PDL20). In some embodiments, the PLA comprises a poly(D,L-lactide) with an inherent viscosity between 3.5 dl/g and 5.5 dl/g (measured in CHCl₃ at 25° C.) (such as the polymer sold under the tradename Purasorb® PDL45). In some embodiments, the PLA comprises a PLDL with an L-lactide to D,L-lactide ratio of about 70:30 with an inherent viscosity between 2.0 dl/g and 2.8 dl/g (measured in CHCl₃ at 25° C.) (such as the polymer sold under the tradename Purasorb® PLDL 7024). In some embodiments, the PLA comprises a PLDL with an L-lactide to D,L-lactide ratio of about 70:30 with an inherent viscosity between 2.3 dl/g and 3.3 dl/g (measured in CHCl₃ at 25° C.) (such as the polymer sold under the tradename Purasorb® PLDL 7028). In some embodiments, the PLA comprises a PLDL with an L-lactide to D,L-lactide ratio of about 70:30 with an inherent viscosity between 3.3 dl/g and 4.3 dl/g (measured in CHCl₃ at 25° C.) (such as the polymer sold under the tradename Purasorb® PLDL 7038). In some embodiments, the PLA comprises a PLDL with an L-lactide to D,L-lactide ratio of about 70:30 with an inherent viscosity between 5.5 dl/g and 6.5 dl/g (measured in CHCl₃ at 25° C.) (such as the polymer sold under the tradename Purasorb® PLDL 7060). In some embodiments, the PLA comprises a PLDL with an L-lactide to D,L-lactide ratio of about 80:20 with an inherent viscosity between 3.3 dl/g and 4.3 dl/g (measured in CHCl₃ at 25° C.) (such as the polymer sold under the tradename Purasorb® PLDL 8038). In some embodiments, the PLA comprises a PLDL with an L-lactide to D,L-lactide ratio of about 80:20 with an inherent viscosity between 5.2 dl/g and 6.3 dl/g (measured in CHCl₃ at 25° C.) (such as the polymer sold under the tradename Purasorb® PLDL 8058).

In some embodiments, the carrier polymer is a PCL. For example, in some embodiments, the PCL has an inherent viscosity of about 1.5 to about 1.9 dl/g (such as the PCL sold under the tradename Purasorb® PC 17, available from Corbion).

### Second Structural Member or Central Member

The structural members are attached to a second structural member, such as a central member. That is one or more structural members may radiate from the central member, for example in a stellate configuration. The second structural member or central member may be an elastomeric member, which allows the gastric residence system to be configurable between a folded condensed configuration (for example, during delivery of the gastric residence system) and an expanded configuration (for example, during gastric residence).

The elastomeric member is made from an elastomeric polymer. The elastomeric polymer is typically not an enteric polymer; however, the central elastomeric polymer can also be made from such an enteric polymer where desirable and practical.

The durometer of the elastomeric member determines the folding force of the dosage form and whether it will remain in the stomach; a preferred range is from about 60 to about 90A. The compression set should be as low as possible to avoid having permanent deformation of the gastric residence system when stored in the capsule in its compacted configuration. A preferred range is about 10 % to about 20% range. Materials that fit these requirements are the QP1 range of liquid silicone rubbers from Dow Corning. In any embodiment with a central elastomer, the QP1-270 (70A durometer) liquid silicone rubber can be used.

Elastomers (also referred to as elastic polymers, elastomeric polymers, or tensile polymers) enable the gastric residence system to be compacted, such as by being folded or compressed, into a form suitable for administration to the stomach by swallowing a container or capsule containing the compacted system. Upon dissolution of the capsule in the stomach, the gastric residence system expands into a shape which prevents passage of the system through the pyloric sphincter of the patient for the desired residence time of the system. Thus, the elastomer must be capable of being stored in a compacted configuration in a capsule for a reasonable shelf life, and of expanding to its original shape, or approximately its original shape, upon release from the capsule. In one embodiment, the elastomer is a silicone elastomer. In one embodiment, the elastomer is formed from a liquid silicone rubber (LSR), such as sold in the Dow Corning QP-1 liquid silicone rubber kit. In one embodiment, the elastomer is cross-linked polycaprolactone. In one embodiment, the elastomer is an enteric polymer, such as those listed in the Enteric Polymer Table (Table 1). In some embodiments, the coupling polymer(s) used in the system are also elastomers. Elastomers are preferred for use as the central polymer in the star-shaped or stellate design of the gastric residence systems.

Examples of elastomers which can be used include silicones, such as those formed using Dow Corning QP-1 kits; urethane-cross-linked polycaprolactones; poly(acryloyl 6-aminocaproic acid) (PA6ACA); poly(methacrylic acid-co-ethyl acrylate) (EUDRAGIT L 100-55); and mixtures of poly(acryloyl 6-aminocaproic acid) (PA6ACA) and poly(methacrylic acid-co-ethyl acrylate) (EUDRAGIT L 100-55).

### Other Coupling Members

Components of the gastric residence system may attached directly or through one or more coupling members. The coupling members may be inactive (i.e., free or substantially free of an agent), but can contain a carrier polymer, which may be the same (or same type) as the carrier polymer contained in an adjacent member (or segment) or a different (or different type) of carrier polymer as the carrier polymer contained in an adjacent member (or segment).

In some embodiments, a coupling member separates a first segment of an arm from a second segment of an arm. For example, in some embodiments, the coupling member separates an active segment of an arm from an inactive segment from an arm. The coupling member separating the two segments may directly interface with the two segments. In some embodiments, the first segment, the second segment, and the coupling member separating the two segments (such as directly interfacing with the two segments) comprises the same carrier polymer, such as PCL, TPU, PLA, or other carrier polymer described herein.

In some embodiments, a coupling member separates an arm from a polymeric linker (such as a time-dependent polymeric linker, an enteric polymeric linker, or a dual time-dependent and enteric polymeric linker). The coupling member separating the polymeric linker from the arm may directly interface with the arm and the polymeric linker. In some embodiments, the coupling member comprises the same (or same type) of carrier polymer as the arm at the interface junction, and/or comprises the same (or same type) of carrier polymer as the polymeric linker (i.e., one or more of the one or more additional polymers in the polymeric linker may be the common carrier polymer or common carrier polymer type). For example, in some embodiments, the arm, the polymeric linker and the coupling member between the arm comprise a PCL. In some embodiments, the arm, the polymeric linker and the coupling member between the arm comprise a TPU. In some embodiments, the arm, the polymeric linker and the coupling member between the arm comprise a PLA.

In some embodiments, a coupling member separates a first polymeric linker from a second polymer linker. The coupling member separating the first polymeric linker from the second polymeric linker may directly interface with both polymeric linkers. In some embodiments, the first and second polymeric linkers and the coupling member between the polymeric linkers have a common polymer (or common type of polymer), such as a PCL, a TPU, or a PLA.

In some embodiments, a coupling member separates a polymeric linker from a second structural member (such as a central elastomeric member). The coupling member may interface directly with both the second structural member and the polymeric linker, for example.

### Exemplary Gastric Residence Systems

The following gastric residence systems are exemplary to better illustrate certain embodiments of the system described herein. As these examples are only exemplary, they are not intended to limit the gastric residence system described herein. One skilled in the art, in view of the provided disclosure, would be able to contemplate additional configurations of the gastric residence system.

In one example of a gastric residence system, the system includes a plurality of structural members comprising an active segment comprising a carrier polymer homogenously mixed with a drug, the arms attached to and radially extending from a central elastomeric member through a time-dependent polymeric linker comprising a pH-independent degradable polymer (such as PLGA) and at least one additional polymer (such as PLA or the carrier polymer), wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising a carrier polymer homogenously mixed with a drug, the arms attached to and radially extending from a central elastomeric member through a time-dependent polymeric linker comprising a pH-independent degradable polymer (such as PLGA) and the carrier polymer; wherein the time-dependent polymeric linker is directly bonded to the segment of the structural member comprising the carrier polymer homogenously mixed with the drug; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member and an active segment comprising a carrier polymer homogenously mixed with a drug, the arms attached to and radially extending from a central elastomeric member through a time-dependent polymeric linker comprising a pH-independent degradable polymer (such as PLGA) and the carrier polymer; wherein the time-dependent polymeric linker is directly bonded to the coupling member; wherein the coupling member separates the active segment from the time-dependent polymeric linker; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising PCL polymer homogenously mixed with a drug, the arms attached to and radially extending from a central elastomeric member through a time-dependent polymeric linker comprising a pH-independent degradable polymer (such as PLGA) and PCL, wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising PCL homogenously mixed with a drug, the arms attached to and radially extending from a central elastomeric member through a time-dependent polymeric linker comprising a pH-independent degradable polymer (such as PLGA) and PCL; wherein the time-dependent polymeric linker is directly bonded to the segment of the structural member comprising the PCL homogenously mixed with the drug; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member comprising PCL and an active segment comprising a carrier polymer homogenously mixed with a drug, the arms attached to and radially extending from a central elastomeric member through a time-dependent polymeric linker comprising a pH-independent degradable polymer (such as PLGA) and PCL; wherein the time-dependent polymeric linker is directly bonded to the coupling member of the structural member; wherein the coupling member separates the active segment from the time-dependent polymeric linker; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU polymer homogenously mixed with a drug, the arms attached to and radially extending from a central elastomeric member through a time-dependent polymeric linker comprising a pH-independent degradable polymer (such as PLGA) and TPU, wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU homogenously mixed with a drug, the arms attached to and radially extending from a central elastomeric member through a time-dependent polymeric linker comprising a pH-independent degradable polymer (such as PLGA) and TPU; wherein the time-dependent polymeric linker is directly bonded to the segment of the structural member comprising the TPU homogenously mixed with the drug; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member comprising TPU and an active segment comprising a carrier polymer homogenously mixed with a drug, the arms attached to and radially extending from a central elastomeric member through a time-dependent polymeric linker comprising a pH-independent degradable polymer (such as PLGA) and TPU; wherein the time-dependent polymeric linker is directly bonded to the coupling member of the structural member; wherein the coupling member separates the active segment from the time-dependent polymeric linker; wherein the time-dependent polymeric linker loses 80% or more of its flexural modulus or breaks after incubation in an aqueous solution at pH 1.6 for 14 days at 37° C.; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising a carrier polymer homogenously mixed with a drug, the arms attached to and radially extending from a central elastomeric member through a time-dependent polymeric linker comprising a pH-independent degradable polymer (such as PLGA) and PLA; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising PCL homogenously mixed with a drug, the arms attached to and radially extending from a central elastomeric member through a time-dependent polymeric linker comprising a pH-independent degradable polymer (such as PLGA) and PCL; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU homogenously mixed with a drug, the arms attached to and radially extending from a central elastomeric member through a time-dependent polymeric linker comprising a pH-independent degradable polymer (such as PLGA) and TPU; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising a carrier polymer homogenously mixed with a drug, the arms attached to and radially extending from a central elastomeric member through a time-dependent polymeric linker comprising a pH-independent degradable polymer (such as PLGA) and PLA; wherein the time-dependent polymeric linker is directly bonded to the segment of the structural member comprising the carrier polymer homogenously mixed with the drug; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising PCL homogenously mixed with a drug, the arms attached to and radially extending from a central elastomeric member through a time-dependent polymeric linker comprising a pH-independent degradable polymer (such as PLGA) and PCL; wherein the time-dependent polymeric linker is directly bonded to the segment of the structural member comprising the PCL homogenously mixed with the drug; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU homogenously mixed with a drug, the arms attached to and radially extending from a central elastomeric member through a time-dependent polymeric linker comprising a pH-independent degradable polymer (such as PLGA) and TPU; wherein the time-dependent polymeric linker is directly bonded to the segment of the structural member comprising the TPU homogenously mixed with the drug; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member and an active segment comprising a carrier polymer homogenously mixed with a drug, the arms attached to and radially extending from a central elastomeric member through a time-dependent polymeric linker comprising a pH-independent degradable polymer (such as PLGA) and PLA; wherein the time-dependent polymeric linker is directly bonded to the coupling member; wherein the coupling member separates the active segment from the time-dependent polymeric linker; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member comprising PCL and an active segment comprising a carrier polymer homogenously mixed with a drug, the arms attached to and radially extending from a central elastomeric member through a time-dependent polymeric linker comprising a pH-independent degradable polymer (such as PLGA) and PCL; wherein the time-dependent polymeric linker is directly bonded to the coupling member; wherein the coupling member separates the active segment from the time-dependent polymeric linker; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising a coupling member comprising TPU and an active segment comprising a carrier polymer homogenously mixed with a drug, the arms attached to and radially extending from a central elastomeric member through a time-dependent polymeric linker comprising a pH-independent degradable polymer (such as PLGA) and TPU; wherein the time-dependent polymeric linker is directly bonded to the coupling member; wherein the coupling member separates the active segment from the time-dependent polymeric linker; and wherein the gastric residence system is retained in the stomach for a period of at least 24 hours. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the time-dependent polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU homogenously mixed with a drug, the arms attached to and radially extending from a central elastomeric member through an enteric polymeric linker comprising an enteric polymer and TPU, wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymer is HMPCAS. Optionally, the enteric polymeric linker comprises a plasticizer, such as about 0.5% to about 20% plasticizer (such as about 0.5% to about 12% plasticizer).

In another example of a gastric residence system, the system includes a plurality of structural members comprising an active segment comprising TPU homogenously mixed with a drug, the arms attached to and radially extending from a central elastomeric member through an enteric polymeric linker comprising an enteric polymer and TPU; wherein the enteric polymeric linker is directly bonded to the active segment comprising TPU; wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymer is HMPCAS. Optionally, the enteric polymeric linker comprises a plasticizer, such as about 0.5% to about 20% plasticizer (such as about 0.5% to about 12% plasticizer).

In another example of a gastric residence system, the system includes a plurality of structural members comprising a coupling member comprising TPU and an active segment comprising a carrier polymer homogenously mixed with a drug, the arms attached to and radially extending from a central elastomeric member through an enteric polymeric linker comprising an enteric polymer and TPU; wherein the enteric polymeric linker is directly bonded to the coupling member; wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymer is HMPCAS. Optionally, the enteric polymeric linker comprises a plasticizer, such as about 0.5% to about 20% plasticizer (such as about 0.5% to about 12% plasticizer).

In another example of a gastric residence system, the system includes a plurality of structural members comprising an active segment comprising a carrier polymer homogenously mixed with a drug, the arms attached to and radially extending from a central elastomeric member through an enteric polymeric linker comprising an enteric polymer and PLGA, wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymeric linker comprises the carrier polymer. In some embodiments, the carrier polymer is PCL and the enteric polymeric linker comprise PCL. In some embodiments, the carrier polymer is TPU and the enteric polymeric linker comprise TPU. In some embodiments, the enteric polymer is HMPCAS. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the enteric polymeric linker comprises a plasticizer, such as about 0.5% to about 20% plasticizer (such as about 0.5% to about 12% plasticizer).

In another example of a gastric residence system, the system includes a plurality of structural members comprising an active segment comprising a carrier polymer homogenously mixed with a drug, the arms attached to and radially extending from a central elastomeric member through an enteric polymeric linker comprising an enteric polymer and PLGA; wherein the enteric polymeric linker is directly bonded to the active segment comprising the carrier polymer; wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymer is HMPCAS. In some embodiments, the enteric polymeric linker comprises the carrier polymer. In some embodiments, the carrier polymer is PCL and the enteric polymeric linker comprise PCL. In some embodiments, the carrier polymer is TPU and the enteric polymeric linker comprise TPU. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the enteric polymeric linker comprises a plasticizer, such as about 0.5% to about 20% plasticizer (such as about 0.5% to about 12% plasticizer).

In another example of a gastric residence system, the system includes a plurality of structural members comprising a coupling member and an active segment comprising a carrier polymer homogenously mixed with a drug, the arms attached to and radially extending from a central elastomeric member through an enteric polymeric linker comprising an enteric polymer and PLGA; wherein the enteric polymeric linker is directly bonded to the coupling member; wherein the polymeric linker loses 80% or more of its flexural modulus or breaks after incubation in an aqueous solution at pH 6.5 for 3 days at 37° C.; and wherein the gastric residence system is retained in the stomach for a period of at least 24 hours. In some embodiments, the enteric polymer is HMPCAS. In some embodiments, the coupling member and the enteric polymeric linker comprise a carrier polymer. In some embodiments, the coupling member and the enteric polymeric linker comprise PCL. In some embodiments, the coupling member and the enteric polymeric linker comprise TPU. In some embodiments, the pH-independent degradable polymer comprises acid-terminated PLGA, ester-terminated PLGA, or a mixture thereof. In some embodiments, the polymeric linker comprises 70% or less PLGA (such as about 30% to about 70% PLGA). Optionally, the enteric polymeric linker comprises a plasticizer, such as about 0.5% to about 20% plasticizer (such as about 0.5% to about 12% plasticizer).

### System Dimensions

The system must be able to adopt a compacted state with dimensions that enable the patient to swallow the system (or for the system to be introduced into the stomach by alternate means, such as a feeding tube or gastrostomy tube). Typically, the system is held in the compacted state by a container such as a capsule. Upon entry into the stomach, the system is then released from the container and adopts an uncompacted state, that is, an expanded conformation, with dimensions that prevent passage of the system through the pyloric sphincter, thus permitting retention of the system in the stomach.

Accordingly, the system should be capable of being placed inside a standard-sized capsule of the type commonly used in pharmacy. Standard capsule sizes in use in the United States are provided in the Capsule Table (Table 2). As these are the outer dimensions of the capsule, and as dimensions will vary slightly between capsule manufacturers, the system should be capable of adopting a configuration which is about 0.5 to 1 mm smaller than the outer diameter shown, and about 1 to 2 mm shorter than the length shown in the Capsule Table (Table 2).

TABLE 2

Capsule Table

| Capsule Size | Outer Diameter (mm) | Length (mm) |
|---|---|---|
| 000 | 9.9 | 26.1 |
| 00 | 8.5 | 23.3 |
| 0 | 7.6 | 21.7 |
| 1 | 6.9 | 19.4 |
| 2 | 6.3 | 18.0 |
| 3 | 5.8 | 15.9 |
| 4 | 5.3 | 14.3 |
| 5 | 4.9 | 11.1 |

Capsules can be made of materials well-known in the art, such as gelatin or hydroxypropyl methylcellulose. In one embodiment, the capsule is made of a material that dissolves in the gastric environment, but not in the oral or esophageal environment, which prevents premature release of the system prior to reaching the stomach.

In one embodiment, the system will be folded or compressed into a compacted state in order to fit into the capsule. Once the capsule dissolves in the stomach, the system will adopt a configuration suitable for gastric retention, for example, in a manner such as that shown in FIGS. 1A-1D.

Once released from the container, the system adopts an uncompacted state with dimensions suitable to prevent passage of the gastric residence system through the pyloric sphincter. In one embodiment, the system has at least two perpendicular dimensions, each of at least 2 cm in length; that is, the gastric residence system measures at least about 2 cm in length over at least two perpendicular directions. In another embodiment, the perimeter of the system in its uncompacted state, when projected onto a plane, has two perpendicular dimensions, each of at least 2 cm in length. The two perpendicular dimensions can independently have lengths of from about 2 cm to about 7 cm, about 2 cm to about 6 cm, about 2 cm to about 5 cm, about 2 cm to about 4 cm, about 2 cm to about 3 cm, about 3 cm to about 7 cm, about 3 cm to about 6 cm, about 3 cm to about 5 cm, about 3 cm to about 4 cm, about 4 cm to about 7 cm, about 4 cm to about 6 cm, about 4 cm to about 5 cm, or about 4 cm to about 4 cm. These dimensions prevent passage of the gastric residence system through the pyloric sphincter. For star-shaped polymers with N arms (where N is greater than or equal to three, such as N = 6), the arms can have dimensions such that the system has at least two perpendicular dimensions, each of length as noted above. These two perpendicular dimensions are chosen as noted above in order to promote retention of the gastric residence system.

### Radiopacity

The systems are optionally radiopaque, so that they can be located via abdominal X-ray if necessary. In some embodiments, one or more of the materials used for construction of the system is sufficiently radiopaque for X-ray visualization. In other embodiments, a radiopaque substance is added to one or more materials of the system, or coated onto one or more materials of the system, or are added to a small portion of the system. Examples of suitable radiopaque substances are barium sulfate, bismuth subcarbonate, bismuth oxychloride, and bismuth trioxide. It is preferable that these materials should not be blended into the polymers used to construct the gastric residence system, so as not to alter drug release from the carrier polymer, or desired properties of other system polymers. Metal striping or tips on a small portion of the system components can also be used, such as tungsten.

### Manufacture of the Gastric Residence System

### Manufacture of System Components

Components of the gastric residence systems, such as structural members (or member segments), linkers, and/or other coupling members, can be manufactured by hot melt extrusion or co-extrusion and/or three dimensional printing. Co-extrusion of components of the gastric residence system can be performed using commercially-available equipment, combined with customized co-extruder plumbing and customized dies for the desired configuration. The initial feedstocks for co-extrusion are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the segment or arm to be manufactured are mixed and pelletized using hot melt extrusion. The polymer pellets thus formed are placed into hoppers above single screw extruders and dried to remove surface moisture. Pellets are gravimetrically fed into individual single-screw extruders, where they are melted and pressurized for co-extrusion.

The appropriate molten polymers are then pumped through custom designed dies with multiple channels where they form the required geometry. The composite polymer block is cooled (water-cooled, air-cooled, or both) and cut or stamped into the desired shape, including, but not limited to, such shapes as triangular prisms, rectangular prisms, or cylinder sections (pie-shaped wedges). Materials are generally extruded to a thickness between about 0.2 mm to about 2.0 mm.

In some embodiments of the invention, producing an entire elongate member, or “arm,” of the gastric residence system by co-extruding the arm is contemplated. In some embodiments of the invention, producing a segment of an elongate member, or “arm,” of the gastric residence system by co-extruding the segment of an arm is contemplated. In some embodiments, an arm or a segment thereof is produced by co-extruding adjacent portions of carrier polymer-agent or carrier polymer-agent salt blend and linker material in a bulk configuration, such as a slab configuration. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

In some embodiments, an arm or a segment thereof is produced by co-extruding adjacent portions of carrier polymer-agent or carrier polymer-agent salt blend and linker material in a bulk configuration, such as a slab configuration, while also co-extruding an additional polymer or polymers within the carrier polymer-agent or carrier polymer-agent salt blend, the linker material, or both the carrier polymer-agent (or agent salt) blend and the linker material. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

A plasticizer, such as propylene glycol, polyethylene glycol (PEG), triethyl butyl citrate (TBC), dibutyl sebacate (DBS), triacetin, triethyl citrate (TEC), a poloxamer (e.g., Poloxamer 407, or “P407”), or D-α-tocopheryl polyethylene glycol succinate, among others, may be included in the coextruded polymer blend, which can aid in cutting the extruded material to the desired size or shape. In some embodiments, the molecular weight of the polyethylene glycol is about 200 Da to about 8,000,000 Da (also referred to as 8000 K or 8000 kDa), for example, about 200 Da to about 400 Da, about 400 Da to about 800 Da, about 800 Da to about 1600 Da, about 1600 Da to about 2500 Da, about 2500 Da to about 5000 Da, about 5000 Da to about 10 K, about 10 K to about 20 K, about 20 K to about 50 K, about 50 K to about 100 K, about 100 K to about 200 K, about 200 K to about 400 K, about 400 K to about 800 K, about 800 K to about 1000 K, about 1000 K to about 2000 K, about 2000 K to about 4000 K, about 4000 K to about 6000 K, or about 6000 K to about 8000 K. In some embodiments, the coextruded polymer may include up to 20% plasticizer, such as up to 18% plasticizer, up to 15% plasticizer, up to 12% plasticizer, up to 10% plasticizer, up to 8% plasticizer, up to 6% plasticizer, up to 4% plasticizer, up to 3% plasticizer, up to 2% plasticizer, or up to 1% plasticizer. In some embodiments, the coextruded polymer includes about 0.5% to about 15% plasticizer, such as about 0.5% to about 1%, about 1% to about 2%, about 2% to about 3%, about 3% to about 5%, about 5% to about 7%, about 7% to about 10%, about 10% to about 12%, about 12% to about 15%, about 15% to about 18%, or about 18% to about 20% plasticizer.

### Assembly of System Components

The various components of the gastric residence system or polymer assemblies can be attached to each other by various methods. One convenient method for attachment is heat welding, which involves heating a first surface on a first component at a first temperature to provide a first heated surface, heating a second surface on a second component at a second temperature to provide a second heated surface, and then contacting the first heated surface with the second heated surface (or equivalently, contacting the second heated surface with the first heated surface). The first temperature may be the same as the second temperature, or the first temperature and the second temperature may be different, depending on the properties of the first and second components to be welded together. Heating of the first surface or of the second surface can be performed by contacting the respective surface with a metal platen (a flat metal plate) at the respective temperature. For ease of manufacture, a dual-temperature platen can be used where a first end of the platen is at the first temperature and a second end of the platen is at the second temperature; the first surface can be pressed against the first end of the platen, the second surface can be pressed against the second end of the platen, and then the platen can be removed and the resulting first heated surface can be contacted with the resulting second heated surface. The contacting heated surfaces are pressed together with some degree of force or pressure to ensure adherence after cooling (the applied force or pressure is optionally maintained during the cooling process). Heat welding is also referred to as heat fusion.

Another method for attachment of the various components of the gastric residence systems, or polymer assemblies, is infrared welding. Infrared welding is performed by contacting a first surface on a first component with a second surface on a second component, and irradiating the region of the contacting surfaces with infrared radiation, while applying force or pressure to maintain the contact between the two surfaces, followed by cooling of the attached components (the applied force or pressure is optionally maintained during the cooling process).

After each welding step, an annealing step can optionally be used to increase the strength of the weld. The welded first and second components can be heat annealed by placing the welded components in an oven set to a third temperature (if the components were welded by heat welding, the third temperature can be the same as the first temperature, the same as the second temperature, or different from the first temperature and second temperature used in heat welding). The welded first and second components can be infrared annealed by irradiating the welded region with infrared radiation. Infrared annealing has the advantage that a localized area can be irradiated, unlike heat annealing in an oven where all of the first and second components will be heated.

Any combination of welding and annealing can be used. Heat welding of components can be followed by heat annealing in an oven of the heat weld; heat welding of components can be followed by infrared annealing of the heat weld; infrared welding of components can be followed by heat annealing in an oven of the infrared weld; or infrared welding of components can be followed by infrared annealing of the infrared weld.

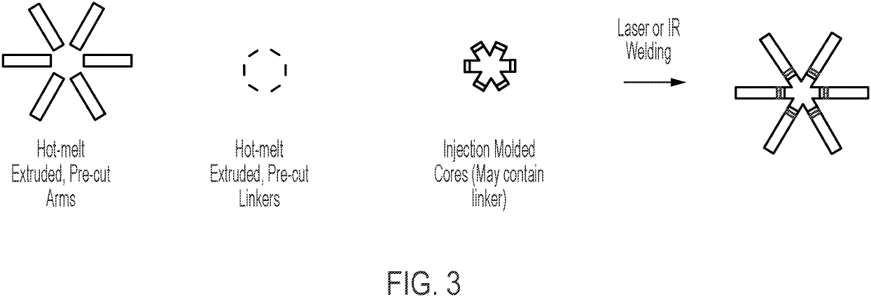

FIG. 3 shows an exemplary method of bonding components together to form a gastric residence system. A pre-cut polymeric linker (such as an enteric linker or a time-dependent linker) is laser or IR welded to an elastomeric central member. The polymeric linker may be formed, for example, by hot melt extruding a material and cutting it to the desired length. Hot melt extruded arms containing a carrier polymer mixed with an agent are then laser or IR welded to the polymeric linkers, thereby forming the stellate structure of the gastric residence system.

Strong attachment of gastric residence system components to each other allows for optimal system performance when deployed in the stomach of an individual. Poor welding or other attachments of system components may cause the interfaces between system components to sever, which can cause some or all of the system to pass through the pyloric valve into the intestine prior completion of the desired gastric residence period. Several features have been identified to enhance attachment of system components, any one or more of which may be utilized in any of the gastric system components, such as the polymeric linkers (e.g., the time-dependent polymeric linker and/or the enteric polymeric linker) described herein.

The inclusion of a plasticizer in a system component may enhance attachment (such as welding) of that component to an immediately adjacent component. For example, a plasticizer may be included in a polymeric linker (such as a time-dependent linker, an enteric linker, or a dual time-dependent and enteric linker) to strengthen the welded interface between the polymeric linker and an immediately adjacent component (such as a structural member comprising a carrier polymer and an agent (or an active or inactive segment thereof), a coupling member, another polymeric linker, or a second structural member (such as an elastomeric central member)). In certain embodiments, too much plasticizer may result in a weaker welded interface compared to a lower amount of plasticizer. Therefore, in some embodiments, the plasticizer in the system component (such as the polymeric linker) is included in an amount of up to 20% plasticizer, such as up to 18% plasticizer, up to 15% plasticizer, up to 12% plasticizer, up to 10% plasticizer, up to 8% plasticizer, up to 6% plasticizer, up to 4% plasticizer, up to 3% plasticizer, up to 2% plasticizer, or up to 1% plasticizer. In some embodiments, the system component (such as the polymeric linker) includes about 0.5% to about 15% plasticizer, such as about 0.5% to about 1%, about 1% to about 2%, about 2% to about 3%, about 3% to about 5%, about 5% to about 7%, about 7% to about 10%, about 10% to about 12%, about 12% to about 15%, about 15% to about 18%, or about 18% to about 20% plasticizer. Exemplary plasticizers include propylene glycol, polyethylene glycol (PEG), triethyl butyl citrate (TBC), dibutyl sebacate (DBS), triacetin, triethyl citrate (TEC), a poloxamer (e.g., Poloxamer 407, or “P407”), and D-α-tocopheryl polyethylene glycol succinate, among others. In some embodiments, the molecular weight of the polyethylene glycol is about 200 Da to about 8,000,000 Da (also referred to as 8000 K or 8000 kDa), for example, about 200 Da to about 400 Da, about 400 Da to about 800 Da, about 800 Da to about 1600 Da, about 1600 Da to about 2500 Da, about 2500 Da to about 5000 Da, about 5000 Da to about 10 K, about 10 K to about 20 K, about 20 K to about 50 K, about 50 K to about 100 K, about 100 K to about 200 K, about 200 K to about 400 K, about 400 K to about 800 K, about 800 K to about 1000 K, about 1000 K to about 2000 K, about 2000 K to about 4000 K, about 4000 K to about 6000 K, or about 6000 K to about 8000 K.

The inclusion of a color-absorbing agent in a system component may enhance attachment (such as welding) of a system component to an immediately adjacent component. The welding includes heading a component, such as using infrared energy. The color-absorbing agent can absorb heat and act as a black body radiation to evenly distribute head to the welded joint, thus enhancing the strength and durability of the weld. Exemplary color-absorbing agents include iron oxide and carbon black.

The inclusion of a common polymer (such as a common carrier polymer) or a common type of polymer (such as a common type of carrier polymer) between joined components of the gastric residence system can enhance the strength of the welded joint between directly adjacent components. By way of example, in some embodiments, a polymeric linker (such as a time-dependent linker, an enteric polymeric linker, or a dual time-dependent and enteric polymeric linker) includes a common polymer or type of polymer with a directly adjacent component (such as a structural member comprising a carrier polymer and an agent (or an active or inactive segment thereof), a coupling member, another polymeric linker, or a second structural member (such as an elastomeric central member)). The common polymer may be, for example, PCL or a type of PCL, a TPU or a type of TPU, or PLA or a type of PLA.

Directly adjacent or welded components may have similar melt flow index at the welding temperature, which can enhance the weld between the joined gastric residence system components. The melt flow index is a measurement of viscosity determined by the grams of material that flow through a capillary in 10 minutes at a set temperature and set load. The melt flow index may be measured, for example, in accordance with the method described in ASTM D1238, using a 2.16 kg load. In some embodiments, the melt flow index of two gastric residence system components welded together differ by no more than 50%, no more than 40%, no more than 30%, no more than 20%, or no more than 10%, relative to the lower melt flow index of the two components. In some embodiments, the weld temperature of the two components is between about 120° C. and about 200° C., such as about 120° C. to about 140° C., about 140° C. to about 160° C., about 160° C. to about 180° C., or about 180° C. to about 200° C.

### Methods of Treatment Using the Gastric Residence Systems

The gastric residence systems can be used to treat conditions requiring administration of a drug or agent over an extended period of time. In a preferred embodiment, a gastric residence system is administered to a human. For long-term administration of agents or drugs which are taken for months, years, or indefinitely, administration of a gastric residence system periodically, such as once weekly or once every two weeks can provide substantial advantages in patient compliance and convenience. Accordingly, the gastric residence systems of the invention can be administered once every three days, once every five days, once weekly, once every ten days, or once every two weeks. The administration frequency is timed to coincide with the designed gastric residence period of the gastric residence system which is administered, so that at about the same time that a gastric residence system passes out of the stomach after its residence period, a new gastric residence system is administered.

Once a gastric residence system has been administered to a patient, the system provides sustained release of agent or drug over the period of gastric retention. After the period of gastric retention, the system degrades and passes out of the stomach. Thus, for a system with a gastric retention period of one week, the patient will swallow (or have administered to the stomach via other methods) a new system every week. Accordingly, in one embodiment, a method of treatment of a patient with a gastric retention system of the invention having a gastric residence period of a number of days D (where D-days is the gastric residence period in days), over a total desired treatment period T-total (where T-total is the desired length of treatment in days) with the agent or drug in the system, comprises introducing a new gastric residence system every D-days into the stomach of the patient, by oral administration or other methods, over the total desired treatment period. The number of gastric residence systems administered to the patient will be (T-total) divided by (D-days). For example, if treatment of a patient for a year (T-total = 365 days) is desired, and the gastric residence period of the system is 7 days (D-days = 7 days), approximately 52 gastric residence systems will be administered to the patient over the 365 days, as a new system will be administered once every seven days.

Alternatively, the patient can swallow (or have administered to the stomach via other methods) a new gastric residence system at the end of the effective release period of the gastric residence system. The “effective release period” or “effective release time” is the time over which the gastric residence system releases an effective amount of the agent contained in the system. Accordingly, in one embodiment, a method of treatment of a patient with a gastric residence system of the invention having an effective release period of a number of days E (where E-days is the effective release period in days), over a total desired treatment period T-total (where T-total is the desired length of treatment in days) with the agent in the system, comprises introducing a new gastric residence system every E-days into the stomach of the patient, by oral administration or other means, over the total desired treatment period. The number of gastric residence systems administered to the patient will be (T-total) divided by (E-days). For example, if treatment of a patient for a year (T-total = 365 days) is desired, and the effective release period of the system is 7 days (E-days = 7 days), approximately 52 gastric residence systems will be administered to the patient over the 365 days, as a new system will be administered once every seven days.

### Kits and Articles of Manufacture

Also provided herein are kits for treatment of patients with the gastric residence systems of the invention. The kit may contain, for example, a sufficient number of gastric residence systems for periodic administration to a patient over a desired total treatment time period. If the total treatment time in days is (T-total), and the gastric residence systems have a residence time of (D-days), then the kit will contain a number of gastric residence systems equal to ((T-total) divided by (D-days)) (rounded to an integral number), for administration every D-days. Alternatively, if the total treatment time in days is (T-total), and the gastric residence systems have an effective release period of (E-days), then the kit will contain a number of gastric residence systems equal to ((T-total) divided by (E-days)) (rounded to an integral number), for administration every E-days. The kit may contain, for example, several gastric residence systems in containers (where the containers may be capsules) and may optionally also contain printed or computer readable instructions for dosing regimens, duration of treatment, or other information pertinent to the use of the gastric residence systems and/or the agent or drug contained in the gastric residence systems. For example, if the total treatment period prescribed for the patient is one year, and the gastric residence system has a residence time of one week or an effective release period of one week, the kit may contain 52 capsules, each capsule containing one gastric residence system, with instructions to swallow one capsule once a week on the same day (e.g., every Saturday).

Articles of manufacture, comprising a sufficient number of gastric residence systems for periodic administration to a patient over a desired total treatment time period, and optionally comprising instructions for dosing regimens, duration of treatment, or other information pertinent to the use of the gastric residence systems and/or the agent or drug contained in the gastric residence systems, are also included in the invention. The articles of manufacture may be supplied in appropriate packaging, such as dispensers, trays, or other packaging that assists the patient in administration of the gastric residence systems at the prescribed interval.

### EXEMPLARY EMBODIMENTS

The following embodiments are exemplary and are not intended to limit the scope of the invention or inventions described herein.

Embodiment 1. A gastric residence system, comprising:
one or more first structural members comprising a carrier polymer and an agent, the one or more first structural members attached to a second structural member through a polymeric linker comprising poly(lactic-co-glycolide) (PLGA) and at least one additional linker polymer;
wherein the polymeric linker loses 20% or more of its flexural modulus or breaks after incubation in an aqueous solution at pH 1.6 for 30 days at 37° C.; and
wherein the gastric residence system is retained in the stomach for a period of at least 24 hours.

Embodiment 2. The gastric residence system of embodiment 1, wherein the polymeric linker loses 40% or more of its flexural modulus or breaks after incubation in an aqueous solution at pH 1.6 for 30 days at 37° C.

Embodiment 3. The gastric residence system of embodiment 1, wherein the polymeric linker loses 60% or more of its flexural modulus or breaks after incubation in an aqueous solution at pH 1.6 for 30 days at 37° C.

Embodiment 4. The gastric residence system of embodiment 1, wherein the polymeric linker loses 80% or more of its flexural modulus or breaks after incubation in an aqueous solution at pH 1.6 for 30 days at 37° C.

Embodiment 5. The gastric residence system of embodiment 1, wherein the polymeric linker loses 90% or more of its flexural modulus or breaks after incubation in an aqueous solution at pH 1.6 for 30 days at 37° C.

Embodiment 6. The gastric residence system of any one of embodiments 1-5, wherein the polymeric linker loses 40% or more of its flexural modulus or breaks after incubation in an aqueous solution at pH 1.6 for 21 days at 37° C.

Embodiment 7. The gastric residence system of any one of embodiments 1-5, wherein the polymeric linker loses 40% or more of its flexural modulus or breaks after incubation in an aqueous solution at pH 1.6 for 21 days at 37° C.

Embodiment 8. The gastric residence system of any one of embodiments 1-5, wherein the polymeric linker loses 60% or more of its flexural modulus or breaks after incubation in an aqueous solution at pH 1.6 for 21 days at 37° C.

Embodiment 9. The gastric residence system of any one of embodiments 1-5, wherein the polymeric linker loses 80% or more of its flexural modulus or breaks after incubation in an aqueous solution at pH 1.6 for 21 days at 37° C.

Embodiment 10. The gastric residence system of any one of embodiments 1-5,wherein the polymeric linker loses 90% or more of its flexural modulus or breaks after incubation in an aqueous solution at pH 1.6 for 21 days at 37° C.

Embodiment 11. The gastric residence system of any one of embodiments 1-10, wherein the polymeric linker loses 20% or more of its flexural modulus or breaks after incubation in an aqueous solution at pH 1.6 for 14 days at 37° C.

Embodiment 12. The gastric residence system of any one of embodiments 1-10, wherein the polymeric linker loses 40% or more of its flexural modulus or breaks after incubation in an aqueous solution at pH 1.6 for 14 days at 37° C.

Embodiment 13. The gastric residence system of any one of embodiments 1-10, wherein the polymeric linker loses 60% or more of its flexural modulus or breaks after incubation in an aqueous solution at pH 1.6 for 14 days at 37° C.

Embodiment 14. The gastric residence system of any one of embodiments 1-10, wherein the polymeric linker loses 80% or more of its flexural modulus or breaks after incubation in an aqueous solution at pH 1.6 for 14 days at 37° C.

Embodiment 15. The gastric residence system of any one of embodiments 1-10, wherein the polymeric linker loses 90% or more of its flexural modulus or breaks after incubation in an aqueous solution at pH 1.6 for 14 days at 37° C.

Embodiment 16. The gastric residence system of any one of embodiments 1-15, wherein the polymeric linker loses 20% or more of its flexural modulus or breaks after incubation in an aqueous solution at pH 1.6 for 7 days at 37° C.

Embodiment 17. The gastric residence system of any one of embodiments 1-15, wherein the polymeric linker loses 40% or more of its flexural modulus or breaks after incubation in an aqueous solution at pH 1.6 for 7 days at 37° C.

Embodiment 18. The gastric residence system of any one of embodiments 1-15, wherein the polymeric linker loses 60% or more of its flexural modulus or breaks after incubation in an aqueous solution at pH 1.6 for 7 days at 37° C.

Embodiment 19. The gastric residence system of any one of embodiments 1-15, wherein the polymeric linker loses 80% or more of its flexural modulus or breaks after incubation in an aqueous solution at pH 1.6 for 7 days at 37° C.

Embodiment 20. The gastric residence system of any one of embodiments 1-15, wherein the polymeric linker loses 90% or more of its flexural modulus or breaks after incubation in an aqueous solution at pH 1.6 for 7 days at 37° C.

Embodiment 21. The gastric residence system of any one of embodiments 1-20, wherein the polymeric linker loses 20% or more of its flexural modulus or breaks after incubation in an aqueous solution at pH 1.6 for 3 days at 37° C.

Embodiment 22. The gastric residence system of any one of embodiments 1-20, wherein the polymeric linker loses 40% or more of its flexural modulus or breaks after incubation in an aqueous solution at pH 1.6 for 3 days at 37° C.

Embodiment 23. The gastric residence system of any one of embodiments 1-20, wherein the polymeric linker loses 60% or more of its flexural modulus or breaks after incubation in an aqueous solution at pH 1.6 for 3 days at 37° C.

Embodiment 24. The gastric residence system of any one of embodiments 1-20, wherein the polymeric linker loses 80% or more of its flexural modulus or breaks after incubation in an aqueous solution at pH 1.6 for 3 days at 37° C.

Embodiment 25. The gastric residence system of any one of embodiments 1-20, wherein the polymeric linker loses 90% or more of its flexural modulus or breaks after incubation in an aqueous solution at pH 1.6 for 3 days at 37° C.

Embodiment 26. The gastric residence system of any one of embodiments 1-25, wherein the at least one additional linker polymer comprises polylactic acid (PLA), the carrier polymer, polycaprolactone (PCL), or a thermoplastic polyurethane (TPU).

Embodiment 27. The gastric residence system of any one of embodiments 1-25, wherein the carrier polymer comprises PCL and the at least one additional linker polymer comprises PCL.

Embodiment 28. The gastric residence system of any one of embodiments 1-25, wherein the carrier polymer comprises TPU and the at least one additional linker polymer comprises a TPU.

Embodiment 29. The gastric residence system of any one of embodiments 1-25, wherein the at least one additional linker polymer comprises PLA.

Embodiment 30. The gastric residence system of embodiment 29, wherein the carrier polymer comprises PCL or TPU.

Embodiment 31. A gastric residence system, comprising:
one or more first structural members comprising a carrier polymer and an agent, the one or more first structural members attached to a second structural member through a polymeric linker comprising:
   (a) poly(lactic-co-glycolide) (PLGA), and
   (b) polylactic acid (PLA), polycaprolactone (PCL), or a thermoplastic polyurethane (TPU); and
wherein the gastric residence system is retained in the stomach for a period of at least 24 hours.

Embodiment 32. The gastric residence system of embodiment 31, wherein the carrier polymer comprises PCL and the polymeric linker comprises the PLGA and the PCL.

Embodiment 33. The gastric residence system of embodiment 31, wherein the carrier polymer comprises the TPU and the polymeric linker comprises the PLGA and the TPU.

Embodiment 34. The gastric residence system of embodiment 31, wherein the polymeric linker comprises the PLGA and the PLA.

Embodiment 35. The gastric residence system of embodiment 34, wherein the carrier polymer comprises the TPU or the PCL.

Embodiment 36. The gastric residence system of any one of embodiments 1-35, wherein the PLGA comprises poly(D,L-lactic-co-glycolide) (PDLG).

Embodiment 37. The gastric residence system of any one of embodiments 1-36, wherein the PLGA comprises acid-terminated PLGA.

Embodiment 38. The gastric residence system of any one of embodiments 1-37, wherein the PLGA comprises ester-terminated PLGA.

Embodiment 39. The gastric residence system of any one of embodiments 1-38, wherein the PLGA comprises acid-terminated PLGA and ester-terminated PLGA at a ratio of about 1:9 to about 9:1.

Embodiment 40. The gastric residence system of any one of embodiments 1-39, wherein the polymeric linker comprises about 70 wt% or less PLGA.

Embodiment 41. The gastric residence system of any one of embodiments 1-40, wherein the polymeric linker comprises between about 30 wt% and about 70 wt% PLGA.

Embodiment 42. The gastric residence system of any one of embodiments 1-41, wherein the polymeric linker further comprises an enteric polymer.

Embodiment 43. The gastric residence system of embodiment 42, wherein the enteric polymer comprises hydroxypropyl methylcellulose acetate succinate (HPMCAS).

Embodiment 44. The gastric residence system of any one of embodiments 1-43, wherein the polymeric linker further loses 20% or more of its flexural modulus or breaks after incubation in an aqueous solution at pH 6.5 for 3 days at 37° C.

Embodiment 45. The gastric residence system of any one of embodiments 1-43, wherein the polymeric linker further loses 40% or more of its flexural modulus or breaks after incubation in an aqueous solution at pH 6.5 for 3 days at 37° C.

Embodiment 46. The gastric residence system of any one of embodiments 1-43, wherein the polymeric linker further loses 60% or more of its flexural modulus or breaks after incubation in an aqueous solution at pH 6.5 for 3 days at 37° C.

Embodiment 47. The gastric residence system of any one of embodiments 1-43, wherein the polymeric linker further loses 80% or more of its flexural modulus or breaks after incubation in an aqueous solution at pH 6.5 for 3 days at 37° C.

Embodiment 48. The gastric residence system of any one of embodiments 1-47, wherein the polymeric linker further loses 20% or more of its flexural modulus or breaks after incubation in an aqueous solution at pH 6.5 for 1 day at 37° C.

Embodiment 49. The gastric residence system of any one of embodiments 1-47, wherein the polymeric linker further loses 40% or more of its flexural modulus or breaks after incubation in an aqueous solution at pH 6.5 for 1 day at 37° C.

Embodiment 50. The gastric residence system of any one of embodiments 1-47, wherein the polymeric linker further loses 60% or more of its flexural modulus or breaks after incubation in an aqueous solution at pH 6.5 for 1 day at 37° C.

Embodiment 51. The gastric residence system of any one of embodiments 1-47, wherein the polymeric linker further loses 80% or more of its flexural modulus or breaks after incubation in an aqueous solution at pH 6.5 for 1 day at 37° C.

Embodiment 52. The gastric residence system of any one of embodiments 1-43, wherein the polymeric linker loses its flexural modulus after incubation in an aqueous solution at pH 6.5 for 3 days at 37° C. by more than 10% of the loss of its flexural modulus after incubation an aqueous solution at pH 1.6 for 3 days.

Embodiment 53. The gastric residence system of any one of embodiments 1-43, wherein the polymeric linker loses its flexural modulus after incubation in an aqueous solution at pH 6.5 for 3 days at 37° C. by more than 20% of the loss of its flexural modulus after incubation an aqueous solution at pH 1.6 for 3 days.

Embodiment 54. The gastric residence system of any one of embodiments 1-43, wherein the polymeric linker loses its flexural modulus after incubation in an aqueous solution at pH 6.5 for 3 days at 37° C. by more than 40% of the loss of its flexural modulus after incubation an aqueous solution at pH 1.6 for 3 days.

Embodiment 55. The gastric residence system of any one of embodiments 1-43, wherein the polymeric linker loses its flexural modulus after incubation in an aqueous solution at pH 6.5 for 3 days at 37° C. by more than 60% of the loss of its flexural modulus after incubation an aqueous solution at pH 1.6 for 3 days.

Embodiment 56. The gastric residence system of any one of embodiments 1-43, wherein the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

Embodiment 57. The gastric residence system of any one of embodiments 1-56, wherein the one or more first structural members are attached to the second structural member through the polymeric linker and a second polymeric linker, the second polymeric linker comprising an enteric polymer.

Embodiment 58. The gastric residence system of embodiment 57, wherein the second polymeric linker further comprises TPU, PCL or PLGA.

Embodiment 59. The gastric residence system of embodiment 57 or 58, wherein the second polymeric linker loses 20% or more of its flexural modulus or breaks after incubation in an aqueous solution at pH 6.5 for 3 days at 37° C.

Embodiment 60. A gastric residence system, comprising:
one or more first structural members comprising a carrier polymer and an agent, the one or more first structural members attached to a second structural member through a polymeric linker comprising:
   (a) a thermoplastic polyurethane (TPU) or comprising poly(lactic-co-glycolide) (PLGA), and
   (b) an enteric polymer;
wherein the polymeric linker loses 20% or more of its flexural modulus or breaks after incubation in an aqueous solution at pH 6.5 for 3 days at 37° C.; and
wherein the gastric residence system is retained in the stomach for a period of at least 24 hours.

Embodiment 61. The gastric residence system of embodiment 60, wherein the polymeric linker further loses 40% or more of its flexural modulus or breaks after incubation in an aqueous solution at pH 6.5 for 3 days at 37° C.

Embodiment 62. The gastric residence system of embodiment 60, wherein the polymeric linker further loses 60% or more of its flexural modulus or breaks after incubation in an aqueous solution at pH 6.5 for 3 days at 37° C.

Embodiment 63. The gastric residence system of embodiment 60, wherein the polymeric linker further loses 80% or more of its flexural modulus or breaks after incubation in an aqueous solution at pH 6.5 for 3 days at 37° C.

Embodiment 64. The gastric residence system of any one of embodiments 60-63, wherein the polymeric linker further loses 20% or more of its flexural modulus or breaks after incubation in an aqueous solution at pH 6.5 for 1 day at 37° C.

Embodiment 65. The gastric residence system of any one of embodiments 60-63, wherein the polymeric linker further loses 40% or more of its flexural modulus or breaks after incubation in an aqueous solution at pH 6.5 for 1 day at 37° C.

Embodiment 66. The gastric residence system of any one of embodiments 60-63, wherein the polymeric linker further loses 60% or more of its flexural modulus or breaks after incubation in an aqueous solution at pH 6.5 for 1 day at 37° C.

Embodiment 67. The gastric residence system of any one of embodiments 60-63, wherein the polymeric linker further loses 80% or more of its flexural modulus or breaks after incubation in an aqueous solution at pH 6.5 for 1 day at 37° C.

Embodiment 68. The gastric residence system of embodiment 60, wherein the polymeric linker loses its flexural modulus after incubation in an aqueous solution at pH 6.5 for 3 days at 37° C. by more than 10% of the loss of its flexural modulus after incubation an aqueous solution at pH 1.6 for 3 days.

Embodiment 69. The gastric residence system of embodiment 60, wherein the polymeric linker loses its flexural modulus after incubation in an aqueous solution at pH 6.5 for 3 days at 37° C. by more than 20% of the loss of its flexural modulus after incubation an aqueous solution at pH 1.6 for 3 days.

Embodiment 70. The gastric residence system of embodiment 60, wherein the polymeric linker loses its flexural modulus after incubation in an aqueous solution at pH 6.5 for 3 days at 37° C. by more than 40% of the loss of its flexural modulus after incubation an aqueous solution at pH 1.6 for 3 days.

Embodiment 71. The gastric residence system of embodiment 60, wherein the polymeric linker loses its flexural modulus after incubation in an aqueous solution at pH 6.5 for 3 days at 37° C. by more than 60% of the loss of its flexural modulus after incubation an aqueous solution at pH 1.6 for 3 days.

Embodiment 72. The gastric residence system of embodiment 60, wherein the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

Embodiment 73. The gastric residence system of any one of embodiments 60-72, wherein the carrier polymer comprises TPU and the one or more polymeric linkers comprises TPU.

Embodiment 74. The gastric residence system of any one of embodiments 60-72, wherein the polymeric linker comprises PLGA.

Embodiment 75. The gastric residence system of embodiment 74, wherein the polymeric linker further comprises polylactic acid (PLA).

Embodiment 76. The gastric residence system of embodiment 74 or 75, wherein the PLGA is poly(D,L-lactic-co-glycolide) (PDLG).

Embodiment 77. The gastric residence system of any one of embodiments 74-76, wherein the PLGA comprises acid-terminated PLGA.

Embodiment 78. The gastric residence system of any one of embodiments 74-77, wherein the PLGA comprises ester-terminated PLGA.

Embodiment 79. The gastric residence system of any one of embodiments 74-78, wherein the PLGA comprises acid-terminated PLGA and ester-terminated PLGA at a ratio of about 1:9 to about 9:1.

Embodiment 80. The gastric residence system of any one of embodiments 74-79, wherein the polymeric linker comprises about 70 wt% or less PLGA.

Embodiment 81. The gastric residence system of any one of embodiments 74-80, wherein the polymeric linker comprises between about 30 wt% and about 70% PLGA.

Embodiment 82. The gastric residence system of any one of embodiments 60-81, wherein the enteric polymer comprises hydroxypropyl methylcellulose acetate succinate (HPMCAS).

Embodiment 83. The gastric residence system of any one of embodiments 60-82, wherein the polymeric linker comprises about 20 wt% to about 80 wt% enteric polymer.

Embodiment 84. The gastric residence system of any one of embodiments 1-83, wherein the polymeric linker comprises about 0.5 wt% to about 20 wt% plasticizer.

Embodiment 85. The gastric residence system of embodiment 84, wherein the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

Embodiment 86. A gastric residence system, comprising:
one or more first structural members comprising a carrier polymer and an agent, the one or more first structural members attached to a second structural member through a polymeric linker comprising a linker polymer and about 0.5 wt% to about 20 wt% plasticizer;
wherein the gastric residence system is retained in the stomach for a period of at least 24 hours.

Embodiment 87. The gastric residence system of embodiment 86, wherein the polymeric linker comprises about 0.5% to about 12% plasticizer.

Embodiment 88. The gastric residence system of embodiment 86 or 87, wherein the linker polymer comprises an enteric polymer.

Embodiment 89. The gastric residence system of embodiment 88, wherein the polymeric linker loses 20% or more of their flexural modulus or breaks after incubation in an aqueous solution at pH 6.5 for 3 days at 37° C.

Embodiment 90. The gastric residence system of embodiment 88, wherein the polymeric linker loses 40% or more of its flexural modulus or breaks after incubation in an aqueous solution at pH 6.5 for 3 days at 37° C.

Embodiment 91. The gastric residence system of embodiment 88, wherein the polymeric linker loses 60% or more of its flexural modulus or breaks after incubation in an aqueous solution at pH 6.5 for 3 days at 37° C.

Embodiment 92. The gastric residence system of embodiment 88, wherein the polymeric linker loses 80% or more of its flexural modulus or breaks after incubation in an aqueous solution at pH 6.5 for 3 days at 37° C.

Embodiment 93. The gastric residence system of any one of embodiments 88-92, wherein the polymeric linker loses 20% or more of its flexural modulus or breaks after incubation in an aqueous solution at pH 6.5 for 1 day at 37° C.

Embodiment 94. The gastric residence system of any one of embodiments 88-92, wherein the polymeric linker loses 40% or more of its flexural modulus or breaks after incubation in an aqueous solution at pH 6.5 for 1 day at 37° C.

Embodiment 95. The gastric residence system of any one of embodiments 88-92, wherein the polymeric linker loses 60% or more of its flexural modulus or breaks after incubation in an aqueous solution at pH 6.5 for 1 day at 37° C.

Embodiment 96. The gastric residence system of any one of embodiments 88-92, wherein the polymeric linker loses 80% or more of its flexural modulus or breaks after incubation in an aqueous solution at pH 6.5 for 1 day at 37° C.

Embodiment 97. The gastric residence system of embodiment 88, wherein the polymeric linker loses its flexural modulus after incubation in an aqueous solution at pH 6.5 for 3 days at 37° C. by more than 10% of the loss of its flexural modulus after incubation an aqueous solution at pH 1.6 for 3 days.

Embodiment 98. The gastric residence system of embodiment 88, wherein the polymeric linker loses its flexural modulus after incubation in an aqueous solution at pH 6.5 for 3 days at 37° C. by more than 20% of the loss of its flexural modulus after incubation an aqueous solution at pH 1.6 for 3 days.

Embodiment 99. The gastric residence system of embodiment 88, wherein the polymeric linker loses its flexural modulus after incubation in an aqueous solution at pH 6.5 for 3 days at 37° C. by more than 40% of the loss of its flexural modulus after incubation an aqueous solution at pH 1.6 for 3 days.

Embodiment 100. The gastric residence system of embodiment 88, wherein the polymeric linker loses its flexural modulus after incubation in an aqueous solution at pH 6.5 for 3 days at 37° C. by more than 60% of the loss of its flexural modulus after incubation an aqueous solution at pH 1.6 for 3 days.

Embodiment 101. The gastric residence system of embodiment 88, wherein the polymeric linker loses its flexural modulus after incubation in an aqueous solution at pH 6.5 for 3 days at 37° C. by more than 80% of the loss of its flexural modulus after incubation an aqueous solution at pH 1.6 for 3 days.

Embodiment 102. The gastric residence system of any one of embodiments 88-101, wherein the enteric polymer comprises hydroxypropyl methylcellulose acetate succinate (HPMCAS).

Embodiment 103. The gastric residence system of any one of embodiments 88-102, wherein the polymeric linker comprises about 20 wt% to about 80 wt% enteric polymer.

Embodiment 104. The gastric residence system of any one of embodiments 86-103, wherein the linker polymer comprises the carrier polymer.

Embodiment 105. The gastric residence system of embodiment 104 wherein the carrier polymer is polycaprolactone (PCL) or a thermoplastic polyurethane (TPU).

Embodiment 106. The gastric residence system of any one of embodiments 86-103, wherein the linker polymer comprises polylactic acid (PLA), polycaprolactone (PCL), or a thermoplastic polyurethane (TPU).

Embodiment 107. The gastric residence system of any one of embodiments 86-106, wherein the linker polymer comprises a time-dependent degradable polymer.

Embodiment 108. The gastric residence system of any one of embodiments 86-107, wherein the polymeric linker loses 20% or more of its flexural modulus or breaks after incubation in an aqueous solution at pH 1.6 for 30 days at 37° C.

Embodiment 109. The gastric residence system of any one of embodiments 86-107, wherein the polymeric linker loses 40% or more of its flexural modulus or breaks after incubation in an aqueous solution at pH 1.6 for 30 days at 37° C.

Embodiment 110. The gastric residence system of any one of embodiments 86-107, wherein the polymeric linker loses 60% or more of its flexural modulus or breaks after incubation in an aqueous solution at pH 1.6 for 30 days at 37° C.

Embodiment 111. The gastric residence system of any one of embodiments 86-107, wherein the polymeric linker loses 80% or more of its flexural modulus or breaks after incubation in an aqueous solution at pH 1.6 for 30 days at 37° C.

Embodiment 112. The gastric residence system of any one of embodiments 86-107, wherein the polymeric linker loses 90% or more of its flexural modulus or breaks after incubation in an aqueous solution at pH 1.6 for 30 days at 37° C.

Embodiment 113. The gastric residence system of any one of embodiments 86-112, wherein the polymeric linker loses 20% or more of its flexural modulus or breaks after incubation in an aqueous solution at pH 1.6 for 21 days at 37° C.

Embodiment 114. The gastric residence system of any one of embodiments 86-112, wherein the polymeric linker loses 40% or more of its flexural modulus or breaks after incubation in an aqueous solution at pH 1.6 for 21 days at 37° C.

Embodiment 115. The gastric residence system of any one of embodiments 86-112, wherein the polymeric linker loses 60% or more of its flexural modulus or breaks after incubation in an aqueous solution at pH 1.6 for 21 days at 37° C.

Embodiment 116. The gastric residence system of any one of embodiments 86-112, wherein the polymeric linker loses 80% or more of its flexural modulus or breaks after incubation in an aqueous solution at pH 1.6 for 21 days at 37° C.

Embodiment 117. The gastric residence system of any one of embodiments 86-112, wherein the polymeric linker loses 90% or more of its flexural modulus or breaks after incubation in an aqueous solution at pH 1.6 for 21 days at 37° C.

Embodiment 118. The gastric residence system of any one of embodiments 86-117, wherein the polymeric linker loses 20% or more of its flexural modulus or breaks after incubation in an aqueous solution at pH 1.6 for 14 days at 37° C.

Embodiment 119. The gastric residence system of any one of embodiments 86-117, wherein the polymeric linker loses 40% or more of its flexural modulus or breaks after incubation in an aqueous solution at pH 1.6 for 14 days at 37° C.

Embodiment 120. The gastric residence system of any one of embodiments 86-117, wherein the polymeric linker loses 60% or more of its flexural modulus or breaks after incubation in an aqueous solution at pH 1.6 for 14 days at 37° C.

Embodiment 121. The gastric residence system of any one of embodiments 86-117, wherein the polymeric linker loses 80% or more of its flexural modulus or breaks after incubation in an aqueous solution at pH 1.6 for 14 days at 37° C.

Embodiment 122. The gastric residence system of any one of embodiments 86-117, wherein the polymeric linker loses 90% or more of its flexural modulus or breaks after incubation in an aqueous solution at pH 1.6 for 14 days at 37° C.

Embodiment 123. The gastric residence system of any one of embodiments 86-122, wherein the polymeric linker loses 20% or more of its flexural modulus or breaks after incubation in an aqueous solution at pH 1.6 for 7 days at 37° C.

Embodiment 124. The gastric residence system of any one of embodiments 86-122, wherein the polymeric linker loses 40% or more of its flexural modulus or breaks after incubation in an aqueous solution at pH 1.6 for 7 days at 37° C.

Embodiment 125. The gastric residence system of any one of embodiments 86-122, wherein the polymeric linker loses 60% or more of its flexural modulus or breaks after incubation in an aqueous solution at pH 1.6 for 7 days at 37° C.

Embodiment 126. The gastric residence system of any one of embodiments 86-122, wherein the polymeric linker loses 80% or more of its flexural modulus or breaks after incubation in an aqueous solution at pH 1.6 for 7 days at 37° C.

Embodiment 127. The gastric residence system of any one of embodiments 86-122, wherein the polymeric linker loses 90% or more of its flexural modulus or breaks after incubation in an aqueous solution at pH 1.6 for 7 days at 37° C.

Embodiment 128. The gastric residence system of any one of embodiments 86-127, wherein the polymeric linker loses 20% or more of its flexural modulus or breaks after incubation in an aqueous solution at pH 1.6 for 3 days at 37° C.

Embodiment 129. The gastric residence system of any one of embodiments 86-127, wherein the polymeric linker loses 40% or more of its flexural modulus or breaks after incubation in an aqueous solution at pH 1.6 for 3 days at 37° C.

Embodiment 130. The gastric residence system of any one of embodiments 86-127, wherein the polymeric linker loses 60% or more of its flexural modulus or breaks after incubation in an aqueous solution at pH 1.6 for 3 days at 37° C.

Embodiment 131. The gastric residence system of any one of embodiments 86-127, wherein the polymeric linker loses 80% or more of its flexural modulus or breaks after incubation in an aqueous solution at pH 1.6 for 3 days at 37° C.

Embodiment 132. The gastric residence system of any one of embodiments 86-127, wherein the polymeric linker loses 90% or more of its flexural modulus or breaks after incubation in an aqueous solution at pH 1.6 for 3 days at 37° C.

Embodiment 133. The gastric residence system of any one of embodiments 107-132, wherein the time-dependent degradable polymer comprises poly(lactic-co-glycolide) (PLGA).

Embodiment 134. The gastric residence system of embodiment 133, wherein the PLGA comprises poly(D,L-lactic-co-glycolide) (PDLG).

Embodiment 135. The gastric residence system embodiment 133 or 134, wherein the PLGA comprises acid-terminated PLGA.

Embodiment 136. The gastric residence system of any one of embodiments 133-135, wherein the PLGA comprises ester-terminated PLGA.

Embodiment 137. The gastric residence system of any one of embodiments 133-136, wherein the PLGA comprises acid-terminated PLGA and ester-terminated PLGA at a ratio of about 1:9 to about 9:1.

Embodiment 138. The gastric residence system of any one of embodiments 133-137, wherein the polymeric linker comprises about 70 wt% or less PLGA.

Embodiment 139. The gastric residence system of any one of embodiments 133-138, wherein the polymeric linker comprises between about 30% and about 70% PLGA.

Embodiment 140. The gastric residence system of any one of embodiments 86-139, wherein the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

Embodiment 141. A gastric residence system, comprising:
one or more first structural members comprising a carrier polymer and an agent, the one or more first structural members attached to a second structural member through a polymeric linker comprising:
   (a) a pH-independent degradable polymer, and
   (b) an enteric polymer;
wherein the gastric residence system is retained in the stomach for a period of at least 24 hours.

Embodiment 142. The gastric residence system of embodiment 141, wherein the polymeric linker further comprises the carrier polymer.

Embodiment 143. The gastric residence system of embodiment 142, wherein the carrier polymer is a TPU or a PCL.

Embodiment 144. The gastric residence system of any one of clams 141-143, wherein the pH-independent degradable polymer comprises PLGA.

Embodiment 145. The gastric residence system of embodiment 144, wherein the PLGA is poly(D,L-lactic-co-glycolide) (PDLG).

Embodiment 146. The gastric residence system of embodiment 144 or 145, wherein the PLGA comprises acid-terminated PLGA.

Embodiment 147. The gastric residence system of any one of embodiments 144-146, wherein the PLGA comprises ester-terminated PLGA.

Embodiment 148. The gastric residence system of any one of embodiments 144-147, wherein the PLGA comprises acid-terminated PLGA and ester-terminated PLGA at a ratio of about 1:9 to about 9:1.

Embodiment 149. The gastric residence system of any one of embodiments 144-148, wherein the polymeric linker comprises about 70 wt% or less PLGA.

Embodiment 150. The gastric residence system of any one of embodiments 144-149, wherein the polymeric linker comprises between about 30 wt% and about 70% PLGA.

Embodiment 151. The gastric residence system of any one of embodiments 141-150, wherein the enteric polymer comprises hydroxypropyl methylcellulose acetate succinate (HPMCAS).

Embodiment 152. The gastric residence system of any one of embodiments 141-151, wherein the polymeric linker comprises about 20 wt% to about 80 wt% enteric polymer.

Embodiment 153. The gastric residence system of any one of embodiments 141-152, wherein the polymeric linker comprises about 0.5 wt% to about 20 wt% plasticizer.

Embodiment 154. The gastric residence system of embodiment 153, wherein the plasticizer comprises propylene glycol, polyethylene glycol (PEG), triethyl butyl citrate (TBC), dibutyl sebacate (DBS), triacetin, triethyl citrate (TEC), a poloxamer, or D-α-tocopheryl polyethylene glycol succinate.

Embodiment 155. The gastric residence system of any one of embodiments 1-154, wherein materials in the polymeric linker are homogenously blended.

Embodiment 156. The gastric residence system of any one of embodiments 1-155, wherein the polymeric linker is substantially free of the agent.

Embodiment 157. The gastric residence system of any one of embodiments 1-156, wherein the polymeric linker further comprises a color-absorbing dye.

Embodiment 158. The gastric residence system of embodiment 157, wherein the color-absorbing dye comprises iron oxide.

Embodiment 159. The gastric residence system of any one of embodiments 1-158, comprising a plurality of first structural members, wherein:
each first structural member is attached to the second structural member through a separate polymeric linker;
the second structural member is an elastic central member;
the gastric residence system is configured to be folded and physically constrained during administration and is configured to assume an open retention shape upon removal of a constraint; and
change between the folded shape and the open retention shape is mediated by the elastic central member that undergoes elastic deformation when the residence structure is in the folded shape and recoils when the gastric residence structure assumes the open retention shape.

Embodiment 160. The gastric system of embodiment 159, wherein the gastric residence system is constrained within a capsule configured to degrade with the stomach.

Embodiment 161. The gastric residence system of any one of embodiments 1-160, wherein the agent is a drug.

Embodiment 162. The gastric residence system of any one of embodiments 1-161, wherein the second structural member is an elastomer.

Embodiment 163. The gastric residence system of any one of embodiments 1-162, wherein the second structural member is a central elastomer, and wherein the one or more first structural members are arms that radially project from the central elastomer.

Embodiment 164. The gastric residence system of any one of embodiments 1-163, wherein the gastric residence system is retained in the stomach for a period of at least 48 hours.

Embodiment 165. The gastric residence system of any one of embodiments 1-164, wherein the gastric residence system is retained in the stomach for a period of at least 3 days.

Embodiment 166. The gastric residence system of any one of embodiments 1-164, wherein the gastric residence system is retained in the stomach for a period of at least 7 days.

Embodiment 167. The gastric residence system of any one of embodiments 1-164, wherein the gastric residence system is retained in the stomach for a period of at least 14 days.

Embodiment 168. The gastric residence system of any one of embodiments 1-164, wherein the gastric residence system is retained in the stomach for a period of at least 30 days.

Embodiment 169. A method of delivering an agent to an individual, comprising deploying the gastric residence system of any one of embodiments 1-168, within the stomach of the individual.

Embodiment 170. The method of embodiment 169, wherein the individual is a human.

Embodiment 171. A gastric residence system comprising one or more first structural members attached to a second structural member through a polymeric linker, the polymeric linker comprising 68 to 72 % by weight poly(lactic-co-glycolide) (PLGA) and 28 to 32 % by weight polylactic acid, wherein the PLGA comprises a lactic acid to glycolic acid ratio of 65:35.

Embodiment 172. A gastric residence system comprising one or more first structural members attached to a second structural member through a polymeric linker, the polymeric linker comprising 68 to 72 % poly(lactic-co-glycolide) PLGA by weight and 28 to 32 % by weight polylactic acid, wherein the PLGA comprises a lactic acid to glycolic acid ratio of 75:25.

Embodiment 173. A gastric residence system comprising one or more first structural members attached to a second structural member through a polymeric linker, the polymeric linker comprising 48 to 52 % poly(lactic-co-glycolide) PLGA by weight and 48 to 52 % by weight polylactic acid (PLA), wherein the PLGA comprises a lactic acid to glycolic acid ratio of 75:25.

Embodiment 174. A gastric residence system comprising one or more first structural members attached to a second structural member through a polymeric linker, the polymeric linker comprising 22 to 26 % poly(lactic-co-glycolide) PLGA by weight, 54 to 58 % by weight polylactic acid (PLA), and 18 to 22 % by weight thermoplastic polyurethane (TPU), wherein the PLGA comprises a lactic acid to glycolic acid ratio of 65:35.

Embodiment 175. A gastric residence system comprising one or more first structural members attached to a second structural member through a polymeric linker, the polymeric linker comprising 22 to 26 % poly(lactic-co-glycolide) PLGA by weight, 54 to 58 % by weight polylactic acid (PLA), and 18 to 22 % by weight thermoplastic polyurethane (TPU), wherein the PLGA comprises a lactic acid to glycolic acid ratio of 75:25.

Embodiment 176. A gastric residence system comprising one or more first structural members attached to a second structural member through a polymeric linker, the polymeric linker comprising 38 to 42 % poly(lactic-co-glycolide) PLGA by weight, 38 to 42 % by weight polylactic acid (PLA), and 18 to 22 % by weight TPU, wherein the PLGA comprises a lactic acid to glycolic acid ratio of 75:25.

Embodiment 177. A gastric residence system comprising one or more first structural members attached to a second structural member through a polymeric linker, wherein a glass transition temperature of the polymeric linker decreases to below body temperature after 7-14 days in an aqueous environment.

### EXAMPLES

The technology as disclosed herein is further illustrated by the following non-limiting examples.

In the following examples, reference to a fasted state simulated gastric fluid (FaSSGF) is used to simulate gastric conditions. The FaSSGF was prepared by dissolving 2 g of NaCl in 0.9 L of purified water, and adjusting the pH of the solution to 1.6 using HCl. The volume was then made up to 1.0 L with purified water at room temperature, and 0.06 g of FaSSGF Biorelevent powder was added to 1 L of the HCl/NaCl solution.

Fasted state intestinal fluid (FaSSIF) was used to simulate intestinal conditions, and was prepared by preparing a buffer solution consisting of 0.21 g of NaOH, 1.98 g of NaH2PO4, and 3.09 g of NaCl in 0.45 L of purified water before adjusting the pH of the solution to 6.5 using 1 N NaOH. The volume was then made up to 0.5 L. Next, 1.12 g FaSSIF Biorelevent powder was added to 0.5 L of buffer and stirred until completely dissolved. The volume was made up to 1 L with buffer at room temperature and then left for 2 hour before use.

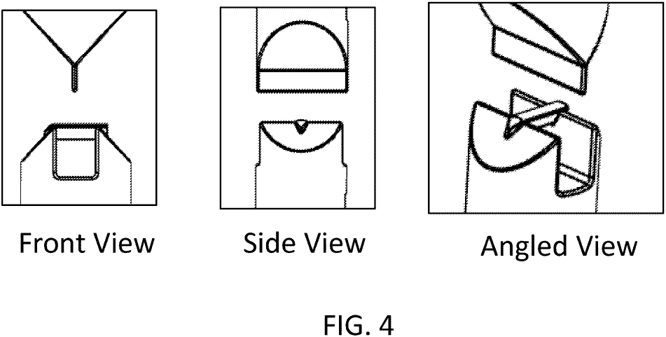

A 3-point bending test in accordance with a modified ASTM D790 standard was used to determine the flexural modulus of the sample. FIG. 4 shows how the flexural modulus of a material may be tested using the three-point bending test. An Instron machine 3342 Series and a custom-made base for triangular shapes were used to evaluate the flexural modulus of samples after incubation in test conditions (e.g., FaSSIF or FaSSGF). To test the flexural modulus of the sample, the sample was placed in the custom-made, with the apex of the triangle facing downward and the flat base facing upward. Pressure was applied to the sample from above and the force (N/mm) was measured using a load cell. Flexural modulus was determined as the slope of the linear region of the force-displacement curve generated by the load cell in units of N/mm.

### Example 1: Time-Dependent Polymeric Linkers

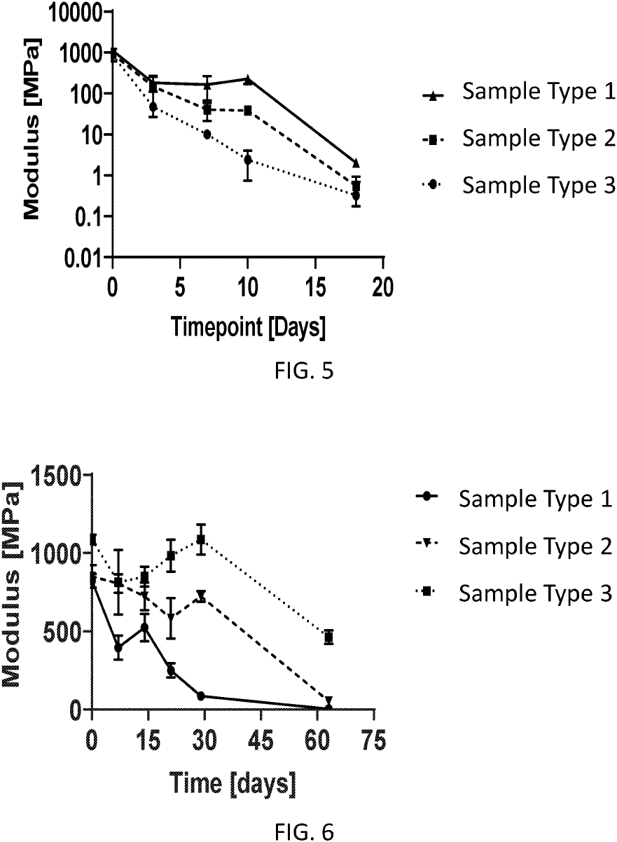

Samples of three time-dependent polymeric linker types containing 85% PLGA and 15% PLA were formed as listed in Table 3. The samples were incubated in FaSSGF at about 37-40° C. for 3, 5, 10, or 18 days before the flexural modulus was measured using a 3-point bending test. Results are shown in FIG. 5, which shows that the sample type 3 degrades faster than sample type 2, which degrades faster than sample type 1, in the FaSSGF.

TABLE 3

| Sample Type No. | 15% PLA | 85% PLGA |
|---|---|---|
| 1 | Purasorb® PLDL 7024 | Resomer® RG 653H |
| 2 | Purasorb® PDL 20 | Resomer® RG 653H |
| 3 | Purasorb® PDL 05 | Resomer® RG 653H |

Also tested were time-dependent polymeric linker samples containing 55% PLGA and 45% PCL, as listed in Table 4. The samples were incubated in FaSSGF at about 37-40° C. for 7, 14, 21, 29, or 63 days before the flexural modulus was measured using a 3-point bending test. Results are shown in FIG. 6, which shows that the sample type 1 degrades faster than sample type 2, which degrades faster than sample type 3, in the FaSSGF.

TABLE 4

| Sample Type No. | 55% PLGA | 45% PCL |
|---|---|---|
| 1 | Purasorb® PDLG 7502 | Purasorb® PC 17 |
| 2 | Purasorb® PDLG 5010 | Purasorb® PC 17 |
| 3 | Purasorb® PDLG 7507 | Purasorb® PC 17 |

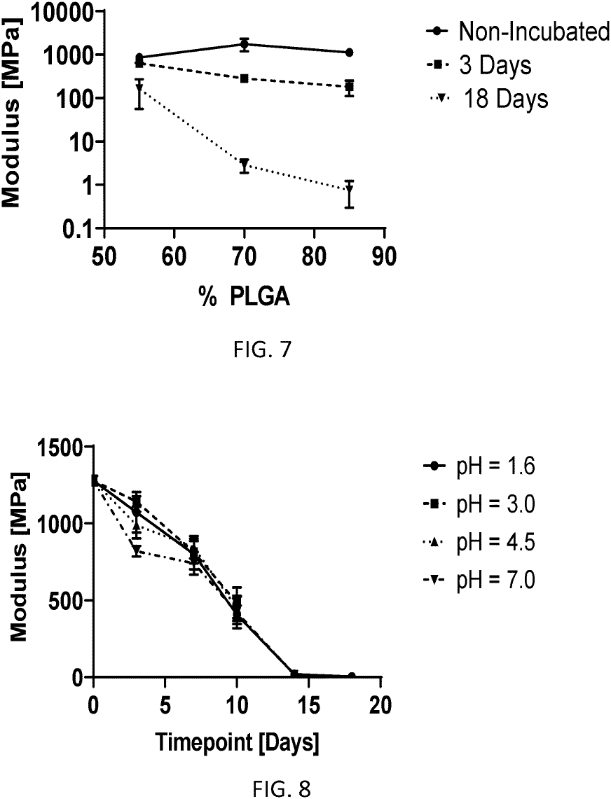

Loss of flexural modulus of the time-dependent polymeric linkers can be adjusted by increasing or decreasing the amount of PLGA polymer in the linker. A higher amount of PLGA results in faster degradation of the sample. Samples containing 55%, 70%, or 85% Resomer® RG 653H (with the balance being Purasorb® PLDL 7024) were incubated for 3 or 18 days in FaSSGF before measuring the flexural modulus. The Results are shown in FIG. 7, which shows that the higher percentage of PLGA in the polymeric linker results in faster degradation under simulated gastric conditions.

The pH independence for the time-dependent polymeric linker was tested by incubating samples of a time-dependent polymeric linker containing PLGA and PCL in an aqueous solution at pH 1.6, 3.0, 4.5, or 7.0 for 3, 7, 10, 14, or 18 days. An exemplary sample contained 44.95% PCL, 53% Purasorb® PDLG 5004A, 2% 100 K polyethylene glycol, and 0.05% iron oxide, and the flexural modulus of the sample after incubation at various pH conditions and lengths of time are shown in FIG. 8. As shown in FIG. 8, the degradation of the sample time-dependent polymeric linker was generally independent of pH, indicating that the PLGA degrades in an aqueous condition independently of the pH and in a time-dependent manner.

### Example 2: Enteric Polymeric Linkers

Enteric polymeric linkers were designed to quickly degrade in the intestine with no or limited degradation in the stomach. An enteric polymer was used to obtain the desired result of the enteric polymeric linker.

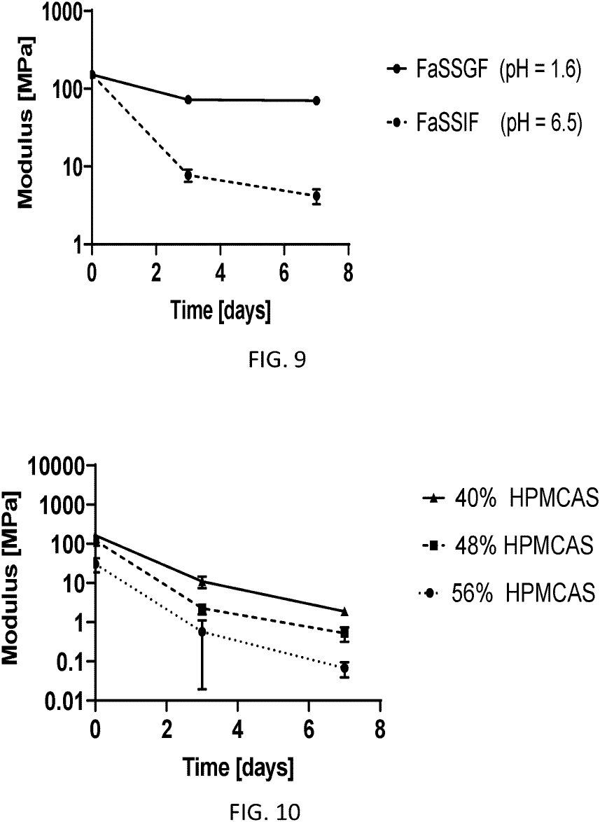

An exemplary enteric polymeric linker was formed by hot melt extrusion of a polymer blend containing 60% HPMCAS MG and 40% Pathways™ 72AE TPU. The flexural modulus o the sample was measured before incubation or after incubation for 3 days or 7 days in either FaSSGF (pH 1.6) or FaSSIF (pH 6.5). The enteric polymeric linker sample substantially degraded in the simulated intestinal conditions (FaSSIF), but did not significantly degrade in the simulated gastric conditions (FaSSGF), as shown in FIG. 9.

Rate of enteric polymeric linker degradation as function of enteric polymer amount in the polymeric linker was tested by forming samples with varying amounts of enteric polymer, as shown in Table 5. The samples were incubated in FaSSIF, and flexural modulus was measured prior to incubation, 3 days after incubation or 7 days after incubation. As shown in FIG. 10, higher amounts of enteric polymer, namely HPMCAS, resulted in faster degradation of the enteric polymeric linker sample in simulated intestinal conditions.

TABLE 5

| Sample Type No. | Enteric Polymer | Carrier Polymer | Additional Components |
|---|---|---|---|
| 1 | 40% HPMCAS MG | 50% Pathways™ 72AE TPU | 10% Propylene Glycol |
| 2 | 48% HPMCAS MG | 40% Pathways™ 72AE TPU | 12% Propylene Glycol |
| 3 | 56% HPMCAS MG | 30% Pathways™ 72AE TPU | 14% Propylene Glycol |

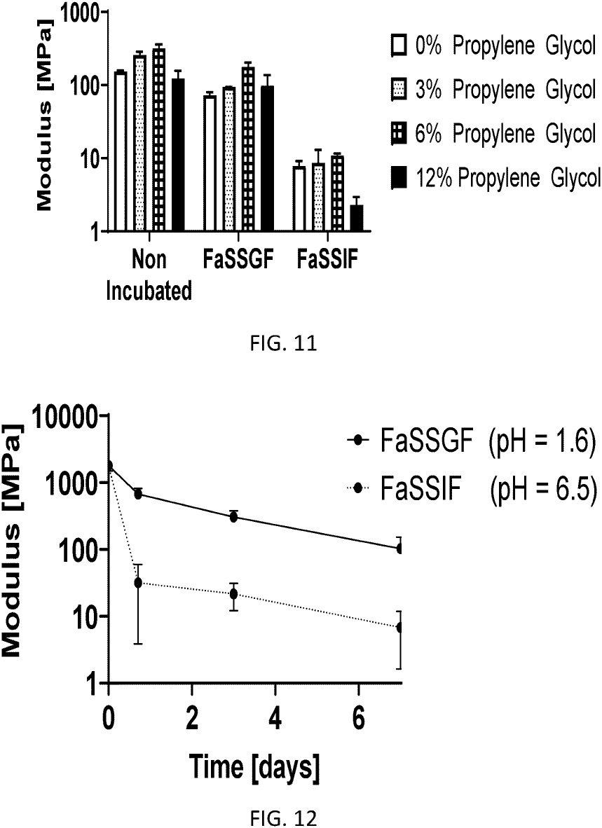

The effect of propylene glycol in the enteric polymeric linker, and its effect on pH dependence, was tested by varying the amount of propylene glycol in the enteric polymeric linker samples, as shone in Table 6, and measuring the change in flexural modulus after incubation for 3 days in simulated gastric conditions (FaSSGF) or simulated intestinal conditions (FaSSIF). Results are shown in FIG. 11, which shows that higher amounts of propylene glycol can enhance degradation of the enteric polymeric linker under simulated intestinal conditions but does not affect the rate of degradation under simulated gastric conditions, even though the samples with higher propylene glycol concentration had lower amounts of enteric polymer (HPMCAS).

TABLE 6

| Sample Type No. | Enteric Polymer | Carrier Polymer | Additional Components |
|---|---|---|---|
| 1 | 60% HPMCAS MG | 40% Pathways™ 72AE TPU | None |
| 1 | 57% HPMCAS MG | 40% Pathways™ 72AE TPU | 3% Propylene Glycol |
| 2 | 54% HPMCAS MG | 40% Pathways™ 72AE TPU | 6% Propylene Glycol |
| 3 | 48% HPMCAS MG | 40% Pathways™ 72AE TPU | 12% Propylene Glycol |

### Example 3: Dual Time-Dependent and Enteric Polymeric Linkers

A dual time-dependent and enteric polymeric linker was formed by including a pH-independent degradable polymer, namely PLGA, with an enteric polymer, namely HPMCAS in a polymeric linker sample. The pH-independent degradable polymer allows for weakening of the polymeric linker at any pH, including gastric conditions, and the enteric polymer allows for accelerated degradation under intestinal conditions.

A dual time-dependent and enteric polymeric linker was formed by hold melt extruding a homogenous mixture of 60% HPMCAS MG and 40% PLGA (namely, Resomer® RG 653H). The flexural modulus of the samples were measured prior to incubation or after incubation for 3 days, 5 days, or 7 days in FaSSGF or FaSSIF. Results are shown in FIG. 12, which demonstrates that the dual time-dependent and enteric polymeric linker degrades slowly in simulated gastric conditions, but quickly in simulated intestinal conditions.

### Example 4: Weld Strength of Linker Materials Joined to Base Polymer

Components of the gastric residence system dosage form were produced through hot melt extrusion, cut to size, and joined together using thermal bonding. The thermal bonding process included loading the selected components into a nest in the desired configuration, applying radial pressure such that all interfaces make contact, and subjecting the exposed side of the components to infrared (IR) radiation. Strong thermal bonds are created when polymer chains are heated to the point at which they can flow across the joint interface and intermingle with chains from the adjacent component. The temperature reached by materials under IR exposure varies between different materials because each polymer blend has its own absorptive and conductive properties. The average process temperature was measured using thermocouples inserted directly into the interface between the two materials.

The material properties were evaluated using a capillary rheometer to determine the melt viscosities in a relevant temperature range. The preliminary viscosity data was used to drive layer reformulation, including adding plasticizers to lower melt viscosity as well as colorants to change IR absorption properties. Bond strength between the layers was evaluated using tensile testing to measure the force required to pull the components apart. This testing was performed on an Instron universal test system using custom grips.

The average peak temperature reached during the process was about 110° C. Variability in these measurements comes from a variety of factors including precise thermocouple positioning - the materials are exposed to IR from one side, so the conductivity of the materials affects how quickly the temperature equilibrates.

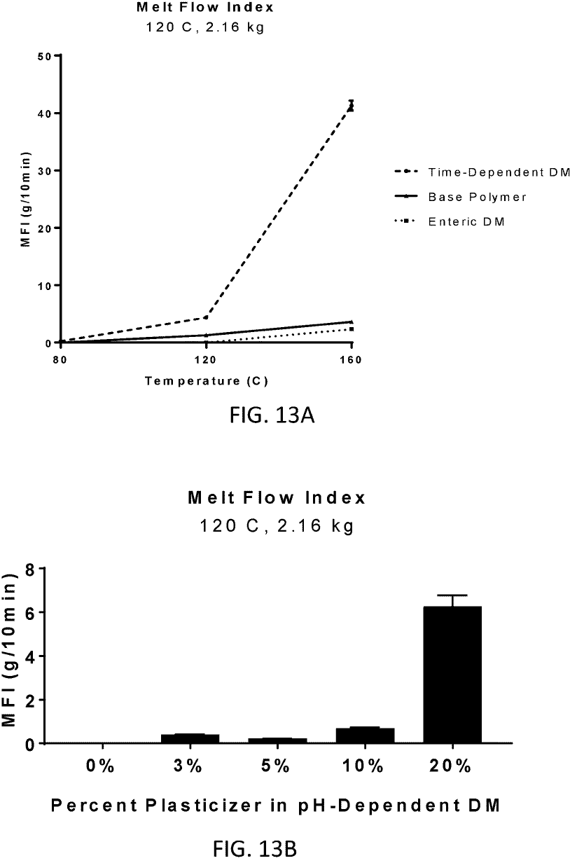

Melt Flow Index. The melt flow index (MFI) is a measurement of viscosity determined by the grams of material that flow through a specific capillary in 10 minutes at a certain temperature and load. Thermal bonds are formed by the intermingling of polymer chains at the layer interfaces, so achieving similar melt flow indices is important for promoting this interaction and creating strong bonds. The two polymeric linker formulations have very different MFIs. An exemplary tested enteric polymeric linker (34% PCL, 64% HPMCAS, 2% P407) does not flow under the 2.16 kg load at all until it is heated to 120° C., whereas an exemplary time-dependent polymeric linker (45% PCL, 35% Purasorb® PDLG 5004A, 18% Purasorb® PDLG 5004, 2% 100 K polyethylene glycol) and the pure carrier polymer (100% PCL) flow significantly more (FIG. 13A). The formulation of the enteric polymeric linker was adjusted as shown in Table 7 to alter the amount of polyethylene glycol, and the melt flow index was measured at 120° C. (FIG. 13B, showing Samples 1-5 of Table 7). As the amount of polyethylene glycol (plasticizer) is increased, so too did the melt flow index.

TABLE 7

| Sample No. | % PCL | % HPMCAS | %P407 | % polyethylene glycol 100 K |
|---|---|---|---|---|
| 1 | 34.00 | 64.00 | 2.00 | 0.00 |
| 2 | 32.98 | 62.08 | 1.94 | 3.00 |
| 3 | 32.30 | 60.80 | 1.90 | 5.00 |
| 4 | 30.60 | 57.60 | 1.80 | 10.00 |
| 5 | 27.20 | 51.20 | 1.60 | 20.00 |
| 6 | 34.00 | 54.30 | 1.70 | 10.00 |
| 7 | 34.00 | 44.61 | 1.39 | 20.00 |

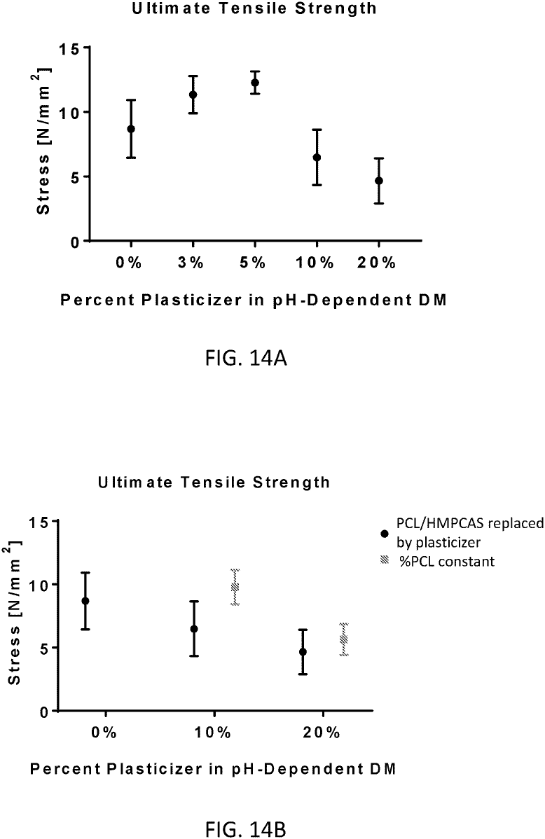

Tensile Strength. An Instron machine and custom-made grips were used to evaluate the ultimate tensile strength (UTS) of the bond between welded materials. The crosshead moves upward at 5-500 mm/minute depending on the elasticity of the materials tested. The instrument records Force (N) v. Displacement (mm), and the maximum force is divided by the cross-sectional area at the interface to calculate ultimate tensile strength (stress). A low ultimate tensile strength indicates a potential failure point in the gastric residence system. The tensile strength of the bond between the enteric polymeric materials listed in Table 7 and the time-dependent linker was measured, as shown in FIG. 14A.

Including a plasticizer in the enteric polymeric linker formulation increased flow at process-relevant temperatures (FIG. 13B) and tensile strength (FIG. 14A) of the bond between the enteric polymeric linker and a joined time-dependent linker. Although including higher amounts of plasticizer in the enteric polymeric linker resulted in a drop in the tensile strength of the bond, this drop can be somewhat recovered by increasing the amount of carrier polymer (e.g., PCL) common to both the enteric polymeric linker and the joined time dependent linker (see FIG. 14B, showing the tensile strength of samples 1, 6 and 7 of Table 7, each having 34% PCL, next to samples 4 and 5 of Table 7 having varying amounts of PCL).

### Example 5: Enteric Polymeric Linkers

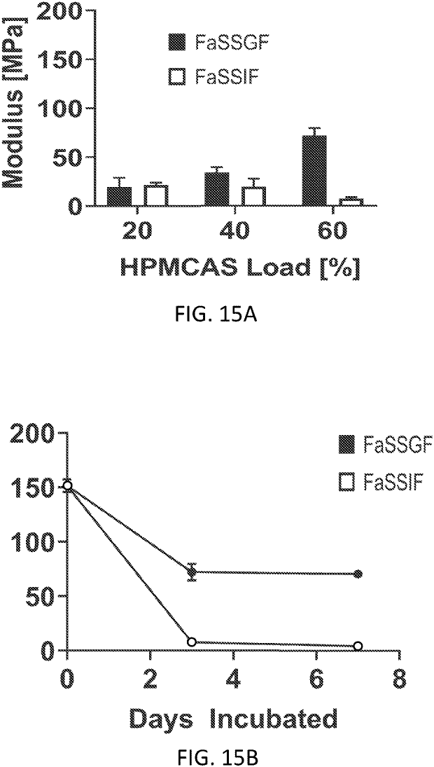

Enteric polymeric linker materials were formed using 20%, 40%, or 60% HPMCAS mixed with 80%, 60%, or 40% Pathways™ 72AE TPU. The polymeric materials were incubated in FaSSIF or FaSSGF at 37° C. for 3 days. The flexural modulus of the materials was measured, which is shown in FIG. 15A. The flexural modulus of the material containing 60% HPMCAS and 40% TPU was measured at 0 days and after 3 days or 7 days incubation in FaSSIF or FaSSGF at 37° C., as shown in FIG. 15B.

The enteric polymeric linker material samples were also cryogenically fractured and incubated in FaSSIF to solubilize the HPMCAS. Scanning electron microscopy (SEM) was performed on samples, and the domains left by the leaching HPMCAS were sized as circles using ImageJ and reported as an Average Domain Size (um), as shown in Table 8. At HPMCAS load 60%, an order of magnitude increase of HPMCAS domain size was observed, leading to improved elution of HPMCAS from the matrix.

TABLE 8

| % HPMCAS | Average Domain Size (um) |
|---|---|
| 20% HPMCAS | 7.68 ± 2.52 |
| 40% HPMCAS | 6.65 ± 3.284 |
| 60% HPMCAS | 60.83 ±3 4.49 |

### Example 6: In Vivo Performance of Polymeric Linkers

Components of the gastric residence systems can be manufactured by various methods, such as co-extrusion or three-dimensional printing, as disclosed in U.S. Pat. No. 10,182,985, and published patent applications US 2018/0311154 A1, US 2019/0262265 A1, US 2019/0231697 A1, US 2019/0254966 A1, and WO 2018/227147.

Gastric residence systems in stellate dosages forms were evaluated in a dog model, a commonly accepted model for preclinical pharmacology and toxicology evaluations. Capsules containing the stellate systems were administered to dogs after fasting for 12 hours. Gastric residence systems were placed in the back of the throat and followed with a food chase. Ventrodorsal X-rays were collected within an hour after dosing and daily for one week. If gastric residence systems were retained in the body longer than one week, X-rays were taken three times per week until the gastric residence systems passed. Six steel fiducials embedded in the gastric residence system enabled analysis of the location (stomach or lower GI tract) and intactness of each gastric residence system.

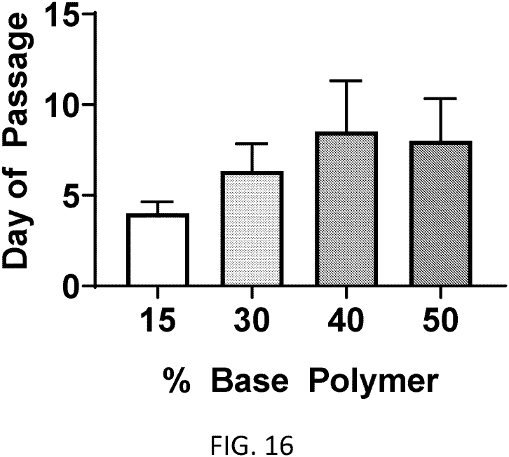

Enteric polymeric linkers containing (a) 15% PCL and 85% HPMCAS, (b) 30% PCL and 70% HPMCAS, (c) 40% PCL and 50% HPMCAS, or (d) 50% PCL and 50% HPMCAS in the stellate dosage forms were tested in the dog model. The enteric polymeric linkers were welded to PCL coupling members of the gastric residence system in the stellate dosage form. Gastric retention in the dog models is shown in FIG. 16, which demonstrates that the dosage forms containing 40% or 50% PCL endured gastric residence for a longer period than enteric linkers with smaller amounts of PCL. The higher amounts of PCL in the enteric polymeric linker enhanced the weld strength of the polymeric linker to the PCL coupling member, which resulted in the longer gastric residence.

Gastric retention in dog models was also tested for additional polymeric linkers using a PCL-based gastric residence system (that is, the polymeric linkers were welded to gastric residence system components containing PCL). The weldability of linker materials to PCL drug arms were determined based on the tensile strength of the bonds, while the gastric retention in a dog model was examined as described above using ventrodorsal X-rays. Enteric characters was measured in vitro by incubating polymeric linker materials in FaSSIF and FaSSGF. If the flexural modulus decreases after incubation in FaSSIF but not in FaSSGF, the enteric character for that material was qualitatively characterized as good (+++ or ++++). If the flexural modulus did not decrease or decreased only slightly, the enteric character for the material was qualitatively characterized as poor (+ or ++). Tested enteric polymeric linkers and results are shown in Table 8, and tested time-dependent polymeric linkers and results are shown in Table 8.

TABLE 9

| Sample | Formulation | | | Gastric Residence in Dogs (average days (StDev)) | Weldability & Adhesion | Enteric Character |
|---|---|---|---|---|---|---|
| | PCL (wt%) | HPMCAS (wt%) | Additives (wt%) | | | |
| 1 | 49.90 | 50 | 0.1% iron oxide | 8.5 (1.2) | +++ | + |
| 2 | 39.90 | 60 | 0.1% iron oxide | 8.5 (2.8) | +++ | + |
| 3 | 38 | 58 | 4% PEO 100 K | 9.5 (3.4) | +++ | + |
| 4 | 35.90 | 36 | 28% TEC 0.1% iron oxide | 4.2 (0.8) | +++ | + |
| 5 | 33.90 | 64 | 2% P407 0.1% iron oxide | 10.0 (2.2) | +++ | ++ |
| 6 | 33.90 | 64 | 2% PEO 100 K 0.1% iron oxide | 6.8 (2.6) | +++ | ++ |
| 7 | 29.90 | 70 | 0.1% iron oxide | 6.0 (1.6) | ++ | +++ |
| 8 | 14.90 | 85 | 0.1% iron oxide | 4.0 (0.6) | + | ++++ |
| 9 | 13.9 | 84 | 2% PEO 100 K 0.1% iron oxide | 4.5 (1.6) | + | ++++ |
| 10 | 14.90 | 75 | 10% TEC 0.1% iron oxide | 2.7 (0.5) | + | ++++ |

High amounts of HPMCAS in the enteric polymeric linker (samples 8-10 in Table 8) provided very good enteric character, but the weld between the enteric polymeric linker and the PCL component was weak, risking breakage and premature gastric exit. The addition of plasticizer in samples with a moderate amount of PCL (samples 3-6 in Table 9) increased the weldability of the polymeric linker to the PCL component. Inclusion of P407 in sample 5 improved cutting of the polymeric linker during product manufacture.

TABLE 10

| Sample | Formulation | | | | Gastric Residence in Dogs (average days (StDev)) | Weldability & Adhesion |
|---|---|---|---|---|---|---|
| | PCL (wt%) | Acid-terminated PLGA (wt%) | Ester-terminated PLGA (wt%) | Additives (wt%) | | |
| 2 | 44.95 | 53 | 0 | 2% PEO 100 K 0.05% iron oxide | 7.6 (0.5) | +++ |
| 3 | 44.95 | 35 | 18 | 2% PEO 100 K 0.05% iron oxide | 8.0 (2.4) | +++ |
| 4 | 50 | 50 | 0 | N/A | 8.5 (2.3) | +++ |
| 5 | 49.95 | 45 | 0 | 5% PEO 100 K 0.05% iron oxide | 4.0 (1.9) | +++ |
| 6 | 50 | 45 | 0 | 5% PEO 100 K | 17.0 (5.9) | N/A |

For the time-dependent polymeric linkers listed in Table 9, all polymeric linkers welded with good strength to the PCL components of the gastric residence system. Addition of 2% PEO resulted in increased flowability of the polymeric mixture during manufacture (sample 2 in Table 9), although adding too much PEO resulted in shortened gastric residence time (sample 5 in Table 9).

Gastric residence systems with one enteric polymeric linker and one time-dependent linker were also tested in dog models. Combination System 1 included a time-dependent polymeric linker according to Sample 2 of Table 9 (average gastric residence of 7.6 days alone), and an enteric polymeric linker according to Sample 3 of Table 8 (average gastric residence of 9.5 days alone), and had an average gastric residence of 8.3 days (2.1 days standard deviation). Combination System 2 included a time-dependent polymeric linker according to Sample 3 of Table 9 (average gastric residence of 8 days) and an enteric polymeric linker according to Sample 5 of Table 8, and had an average gastric residence of 8.5 days (standard deviation 1.5 days). Combination System 3 included a time-dependent polymeric linker according to Sample 2 of Table 9 (average gastric residence of 7.6 days) and an enteric polymeric linker according to Sample 5 of Table 8 (average gastric residence of 4.0 days), and had an average gastric residence of 3.7 days (1.2 days standard deviation).

### Example 7: Mechanical Testing of Disintegrating Matrices Under Various Conditions

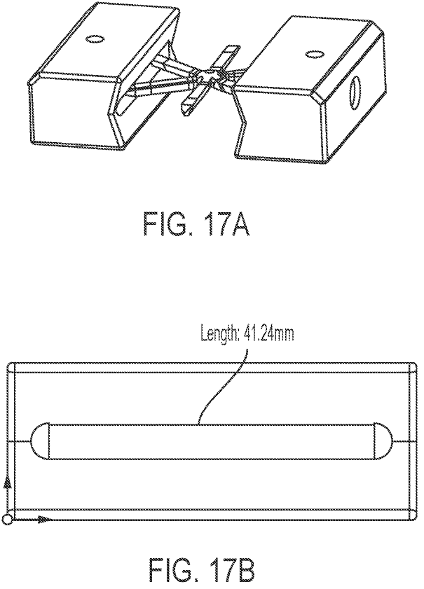

Cyclic Incubated Nonplanar Compressive (CINC) Testing of Disintegrating Matrices: The cyclic incubated nonplanar compressive (CINC) test apparatus was designed to hold stellates submerged in heated aqueous fluid. While submerged, the stellates are compressed by means of two opposing holders, as shown in FIG. 17A. Each of these holders consists of a channel 41.24 mm in length (see FIG. 17B). Both channels are in the horizontal plane, facing each other. The stellate is placed in the apparatus with the ends of two arms in each holder, supporting the stellate with four opposing arms, leaving two arms unsupported. While the holders are fully open, the stellate is in the same (horizontal) plane as the holder channels. During actuation one holder is moved toward the other, causing the stellate to be compressed. The force of the stellate core holds the stellate in the channel as the holders reciprocate. The tips of the arms are free to move within the channels, allowing the angle between the arms to change. With the exception of the tips of the arms, the stellate is not constrained in the vertical plane.

Stellates were incubated in jars that contained fasted state simulated gastric fluid (FaSSGF) at 37° C. At each timepoint stellates were removed from the jars, blotted dry, then photographed. The CINC test apparatus was filled with FaSSGF and preheated to 37° C. Stellates were placed in the CINC test apparatus and allowed to equilibrate for 10 minutes. Stellates were then run in the CINC test apparatus for 50 cycles. Each cycle consisted of a 1.88 second hold with the holders in the open position, a 0.85 second move to the closed position, a 1.88 second hold in the closed position, then 0.85 seconds to return to the open position. In the open position the deepest point of the channels was 38.64 mm apart, and in the closed position the channels were 21.64 mm apart. Immediately following CINC testing, stellates were blotted dry and photographed.. Stellates are observed for signs of failure (bending, weld separation, arm breakage) before and after each compression cycle. Stellates were then returned to the jars of FaSSGF incubated at 37° C. until the next timepoint Stellates are tested by the same procedure until they fail or become too damaged to be held by the fixture. Failure modes and timing of failures (day, number of compression cycles) are reported.

Table 9 shows the results of representative linkers upon testing in CINC. Qualitative results can rank expected gastric residency.

TABLE 9

| Stellate with: | Day Linker failed in CINC Test | Failure mode |
|---|---|---|
| Timing Linker 1 | 2 | Linker bent irreversibly |
| Timing Linker 2 | 14 | Linker bent irreversibly |
| Timing Linker 3 | 30 | Arm broke at linker interface |

Stress Relaxation “Window” Testing: Stress relaxation of linkers within stellates was evaluated using “window” testing, which measures material deformation and recovery of stellate arms after prolonged compression with incubation biorelevant media.

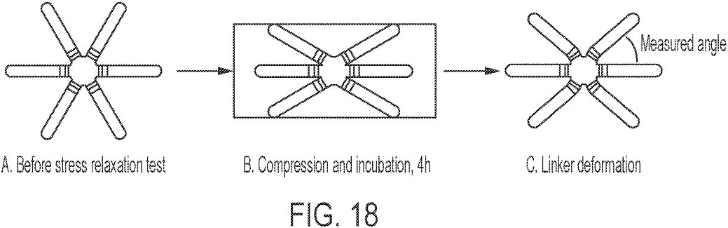

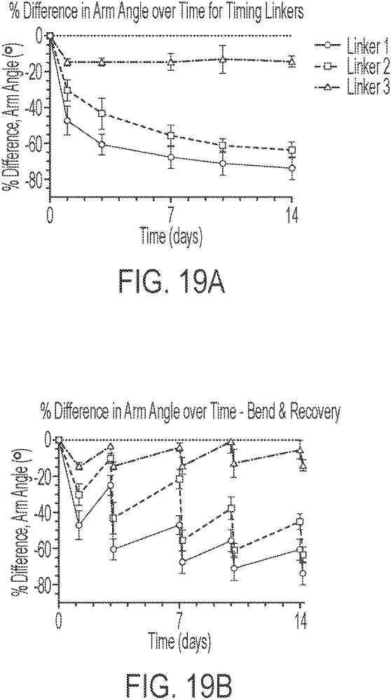

Stellates assembled with linkers (see FIG. 18, panel A) were incubated in biorelevant media (FaSSGF or FaSSIF) at 37° C. prior to testing. At each time point, stellates were photographed and then manually compressed and placed within a compartment of the plastic “window” fixture, which holds stellates in a compressed position during incubation (as in FIG. 18, panel B). The fixtures containing compressed stellates were placed within a sealed container containing biorelevant media at 37° C. for four hours (see again FIG. 18, panel B). Stellates were then removed from the fixture, photographed, and returned to biorelevant media for incubation (without compression) until the next time point. Arm angle change was measured using image analysis software such as ImageJ and was reported as the angle between a bent arm and the neighboring unstressed arm (see FIG. 18, panel C). Results for three different linkers are shown in FIG. 19A and FIG. 19B.

The window fixture used included an array of compartments that were each 50 mm long x 15 mm wide x 15 mm deep. The fixture was 3D-printed with a clear photopolymer resin using a Formlabs Form 2 printer but can be made of any durable material.

For the three different linkers described in Table 11, FIG. 19A displays the % difference in arm angle post-window test over time in the stellate arms, while FIG. 19B also includes the % difference in arm angle after recovery. This data demonstrates clear distinctions in stellate and thus linker behavior.

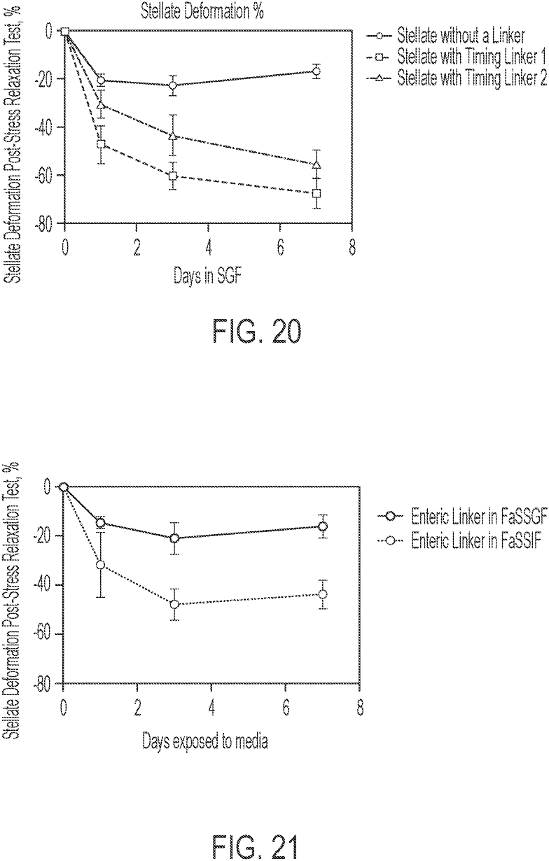

Stellate Deformation Post Stress Relaxation: Stellate Deformation Post Stress Relaxation Time over days is shown in FIG. 20. Stellates with a timing linker demonstrate a time-dependent, tunable stress-relaxation behavior. The profile outlined for Timing Linker 1 is associated with a gastric residence of 7.2 ± 3.2 days, and the profile outlined for Timing Linker 2 is associated with a gastric residence of 19.3 ± 3.9 days. FIG. 21 shows a Stellate Deformation Post-Stress Relaxation Test over days in FaSSGF vs. FaSSIF. This data was collected with representative Enteric Linker 1.

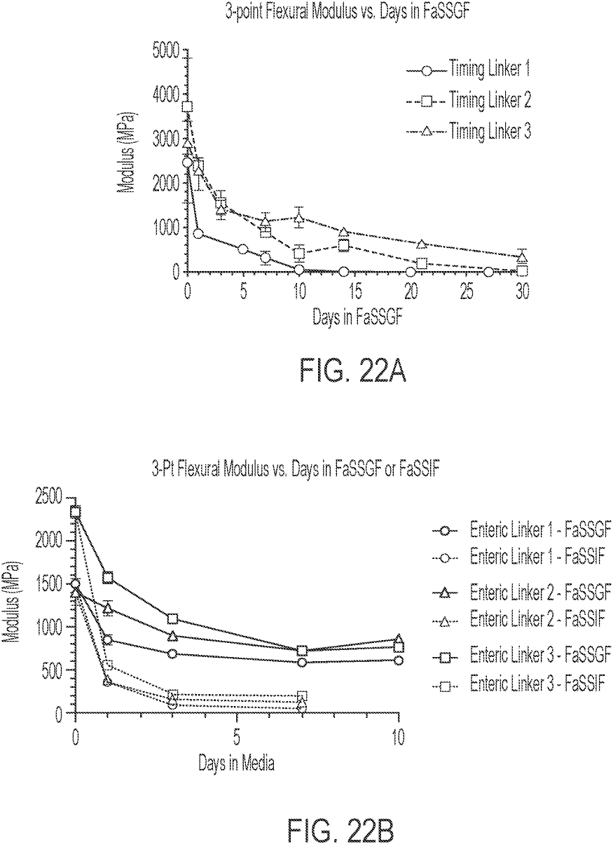

3-Point Bending Test: Tests of 3-Pt of Bending of Enteric and Timing Matrices, which demonstrate the decay of representative timing and enteric linkers in relevant media, are shown in FIG. 22A (timing linkers) and FIG. 22B (enteric linkers).

Table 10 shows a comparison of representative timing linkers in the 3-Pt Bending test, stress relaxation test, CINC test, and their relationship of these parameters with gastric residence. Mechanical tests that capture bending, deformation, and failure of linkers are analyzed together to predict rank order of duration of gastric residence for stellates incorporating different linker formulations.

TABLE 10

| Component | IV [dl/g] | End-cap | DL:G Ratio | Timing Linker 1 | Timing Linker 2 | Timing Linker 3 |
|---|---|---|---|---|---|---|
| PDLG 1 | 0.3 | Acid | 65:35 | 70% | - | - |
| PDLG 2 | 0.3 | Acid | 75:25 | - | 70% | 50% |
| PLDL | 2.4 | Acid | n/a | 30% | 30% | 50% |
| Test | | | | - | - | - |
| Flexural Modulus (3-Point Bend): Days until strength drops below 200 MPa | | | | 7-10 | 10-14 | >30 |
| Stress Relaxation Test: Days into the test that stellate is deformed to >65% | | | | 3-7 | 14-17 | >30 |
| Performance under Cyclic Stress (CINCT): Days until DM failure | | | | 2 | 14 | 30 |
| Gastric residence time in dogs (Days) | | | | 7.2 ± 3.2 | 19.3 ± 3.9 | 25.3 ± 5.2 |

Table 11 shows data for a representative enteric linker in the 3-Pt bending and stress relaxation test. Mechanical tests that capture softening and deformation of enteric linkers in different pH media are used to evaluate pH responsiveness. While all three linkers described maintain stiffness at gastric pH for >10 days, enteric linkers 1 and 2 soften more readily at intestinal pH than enteric linker 3, and therefore may be expected to soften more readily in the intestine.

TABLE 11

| Test | Enteric Linker 1 | Enteric Linker 2 | Enteric Linker 3 |
|---|---|---|---|
| Formulation | 34% PCL 64% HPMCAS 2% P-407 | 40% PDL 20 40% HPMCAS 20% TPU | 55% PDL 20 40% HPMCAS 5% TPU |
| Flexural Modulus (3-Point Bend): Days until strength drops below 200 MPa in FaSSGF | > 10 | > 10 | >10 |
| Flexural Modulus (3-Point Bend): Days until strength drops below 200 MPa in FaSSIF | 1-3 | 1-3 | 3-7 |

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A gastric residence system, comprising:
one or more first structural members comprising a carrier polymer and an agent, the one or more first structural members attached to a second structural member through a polymeric linker comprising poly(lactic-co-glycolide) (PLGA) and at least one additional linker polymer;
wherein the polymeric linker loses 20% or more of its flexural modulus or breaks after incubation in an aqueous solution at pH 1.6 for 30 days at 37° C.; and
wherein the gastric residence system is retained in the stomach for a period of at least 24 hours.

2-5. (canceled)

6. The gastric residence system of claim 1, wherein the polymeric linker loses 40% or more of its flexural modulus or breaks after incubation in an aqueous solution at pH 1.6 for 21 days at 37° C., the polymeric linker loses 20% or more of its flexural modulus or breaks after incubation in an aqueous solution at pH 1.6 for 14 days at 37° C., the polymeric linker loses 20% or more of its flexural modulus or breaks after incubation in an aqueous solution at pH 1.6 for 7 days at 37° C., and the polymeric linker loses 20% or more of its flexural modulus or breaks after incubation in an aqueous solution at pH 1.6 for 3 days at 37° C.

7-25. (canceled)

26. The gastric residence system of claim 1, wherein the at least one additional linker polymer comprises polylactic acid (PLA), the carrier polymer, polycaprolactone (PCL), or a thermoplastic polyurethane (TPU).

27. The gastric residence system of claim 1, wherein the carrier polymer comprises PCL and the at least one additional linker polymer comprises PCL, the carrier polymer comprises TPU and the at least one additional linker polymer comprises a TPU, or the carrier polymer comprises PCL or TPU and the at least one additional linker polymer comprises PLA.

28-35. (canceled)

36. The gastric residence system of claim 1, wherein the PLGA comprises one or more of poly(D,L-lactic-co-glycolide) (PDLG), acid-terminated PLGA, or ester-terminated PLGA.

37-59. (canceled)

60. A gastric residence system, comprising:
one or more first structural members comprising a carrier polymer and an agent, the one or more first structural members attached to a second structural member through a polymeric linker comprising:
(a) a thermoplastic polyurethane (TPU) or comprising poly(lactic-co-glycolide) (PLGA), and
(b) an enteric polymer;
wherein the polymeric linker loses 20% or more of its flexural modulus or breaks after incubation in an aqueous solution at pH 6.5 for 3 days at 37° C.; and
wherein the gastric residence system is retained in the stomach for a period of at least 24 hours.

61. The gastric residence system of claim 60, wherein the polymeric linker further loses 40% or more of its flexural modulus or breaks after incubation in an aqueous solution at pH 6.5 for 3 days at 37° C., the polymeric linker further loses 20% or more of its flexural modulus or breaks after incubation in an aqueous solution at pH 6.5 for 1 day at 37° C., and the polymeric linker loses its flexural modulus after incubation in an aqueous solution at pH 6.5 for 3 days at 37° C. by more than 10% of the loss of its flexural modulus after incubation an aqueous solution at pH 1.6 for 3 days.

62-81. (canceled)

82. The gastric residence system of claim 60, wherein the enteric polymer comprises hydroxypropyl methylcellulose acetate succinate (HPMCAS).

83-85. (canceled)

86. A gastric residence system, comprising:
one or more first structural members comprising a carrier polymer and an agent, the one or more first structural members attached to a second structural member through a polymeric linker comprising a linker polymer and about 0.5 wt% to about 20 wt% plasticizer;
wherein the gastric residence system is retained in the stomach for a period of at least 24 hours.

87. The gastric residence system of claim 86, wherein the polymeric linker comprises about 0.5% to about 12% plasticizer and an enteric polymer comprising hydroxypropyl methylcellulose acetate succinate (HPMCAS).

88. (canceled)

89. The gastric residence system of claim 87, wherein the polymeric linker loses 20% or more of their flexural modulus or breaks after incubation in an aqueous solution at pH 6.5 for 3 days at 37° C., the polymeric linker loses 20% or more of its flexural modulus or breaks after incubation in an aqueous solution at pH 6.5 for 1 day at 37° C., the polymeric linker loses 20% or more of its flexural modulus or breaks after incubation in an aqueous solution at pH 1.6 for 30 days at 37° C., the polymeric linker loses 20% or more of its flexural modulus or breaks after incubation in an aqueous solution at pH 1.6 for 21 days at 37° C., the polymeric linker loses 20% or more of its flexural modulus or breaks after incubation in an aqueous solution at pH 1.6 for 14 days at 37° C., and the polymeric linker loses 20% or more of its flexural modulus or breaks after incubation in an aqueous solution at pH 1.6 for 7 days at 37° C., and the polymeric linker loses 20% or more of its flexural modulus or breaks after incubation in an aqueous solution at pH 1.6 for 3 days at 37° C.

90-154. (canceled)

155. The gastric residence system of claim 1, wherein materials in the polymeric linker are homogenously blended.

156. The gastric residence system of claim 1, wherein the polymeric linker is substantially free of the agent.

157. The gastric residence system of claim 1, wherein the polymeric linker further comprises a color-absorbing dye.

158. The gastric residence system of claim 157, wherein the color-absorbing dye comprises iron oxide.

159. The gastric residence system of claim 1, comprising a plurality of first structural members, wherein:
each first structural member is attached to the second structural member through a separate polymeric linker;
the second structural member is an elastic central member;
the gastric residence system is configured to be folded and physically constrained during administration and is configured to assume an open retention shape upon removal of a constraint; and
change between the folded shape and the open retention shape is mediated by the elastic central member that undergoes elastic deformation when the residence structure is in the folded shape and recoils when the gastric residence structure assumes the open retention shape.

160. The gastric system of claim 159, wherein the gastric residence system is constrained within a capsule configured to degrade with the stomach and the agent is a drug.

161. (canceled)

162. The gastric residence system of claim 1, wherein the second structural member is an elastomer.

163. The gastric residence system of claim 1, wherein the second structural member is a central elastomer, and wherein the one or more first structural members are arms that radially project from the central elastomer.

164-168. (canceled)

169. A method of delivering an agent to an individual, comprising deploying the gastric residence system of claim 1, within the stomach of the individual, wherein the individual is a human.

170-177. (canceled)
